(19) United States
(12) Patent Application Publication
(10) Pub. No.: US 2008/0081037 A1
(43) Pub. Date: Apr. 3, 2008

(54) Methods and Agents for Diagnosis and Prevention, Amelioration or Treatment of Goblet Cell-Related Disorders

(76) Inventors: Johannes Grosse, Martinsried (DE); Boris Schneider, Martinsried (DE); Lutz Zeitlmann, Martinsried (DE); Andreas Popp, Martinsried (DE)

Correspondence Address: FISH & RICHARDSON PC, P.O. BOX 1022, MINNEAPOLIS, MN, 55440-1022, US

(21) Appl. No.: 10/540,818

(22) PCT Filed: Dec. 23, 2003
(86) PCT No.: PCT/EP03/14834

§ 371 Date: Jan. 23, 2006

Related U.S. Application Data
(60) Provisional application No. 60/436,322, filed on Dec. 23, 2002.

(51) Int. Cl. A61K 31/70; A01K 67/00; A61K 39/395; A61P 1/00; C07K 14/00; C12N 1/20; C12N 15/00; C12N 15/11; C12N 5/06; C12Q 1/02; C12Q 1/68; G01N 33/53

(52) U.S. Cl. 424/130.1; 435/252.3; 435/29; 435/320.1; 435/325; 435/6; 435/69.1; 435/7.1; 514/12; 514/2; 514/44; 530/350; 536/23.5; 536/24.5; 800/13; 800/16; 800/18; 800/3; 800/8; 800/9

(57) ABSTRACT

The present invention inter alia relates to methods for the prevention, amelioration or treatment of medical conditions associated with an alteration in normal goblet cell function. It also relates to methods of screening for disease-relevant markers indicative of an increased risk of a subject of developing such a condition. It furthermore relates to an animal model useful for studying said conditions and the molecular mechanisms underlying them, and uses of that animal model, for example for the identification of diagnostic markers or agents useful for the prevention, amelioration, or treatment of a goblet cell-related disorder. Novel agents useful in the above methods, and novel pharmaceutical compositions are likewise provided. The invention further relates to screening methods for agonists and antagonists useful for performing said methods.

### FIELD OF THE INVENTION

The present invention inter alia relates to methods for the prevention, amelioration or treatment of medical conditions associated with an alteration in normal goblet cell function. It also relates to methods of screening for disease-relevant markers indicative of an increased risk of a subject of developing such a condition, as well as to methods of screening for and diagnosis of a predisposition in a human subject for such conditions. It furthermore relates to an animal model useful for studying said medical conditions and the molecular mechanisms underlying it, and uses of that animal model, for example for the identification of diagnostic markers or agents useful for the prevention, amelioration, or treatment of a goblet cell-related disorder.

Novel agents such as polypeptides and fragments thereof, nucleic acids and antibodies which are useful in the above methods, and novel pharmaceutical compositions are likewise provided. The invention further relates to screening methods for agonists and antagonists useful for performing said methods. These and further aspects of the invention will be described in more detail below.

### BACKGROUND OF THE INVENTION

The epithelial mucosal layer is a physical and chemical barrier important in protecting the animal body from dryness, harmful exogenous substances and pathogens. Mucus forms a gel layer covering the epithelial surface, acting as a semi-permeable barrier between the epithelium and the exterior environment. Mucus serves many functions, including protection against shear stress and chemical damage, and, especially in the respiratory tree, trapping and elimination of particulate matter and microorganisms. The mucus layer on top of the intestinal epithelium is the barrier between the host's internal milieu and gut bacteria. In the vertebrate eye, the inner layer of the tear film consists of mucous secretion products. Mucus is a viscous fluid composed primarily of highly glycosylated proteins called mucins suspended in a solution of electrolytes (Dekker et al., 2002). Mucins and other components of mucus are secreted from the apical surface of specialized columnar epithelial cells referred to as goblet cells (Verdugo, 1990).

Goblet cells are distributed among other cells in the epithelium of many organs, especially in the intestinal and respiratory tracts. In areas like the conjunctiva, their numbers are rather small compared to other cell types, whereas in tissues such as the colon, they are much more abundant Goblet cells have a characteristic morphology, based on membrane-bound secretory granules, which contain mucus (Specian and Oliver, 1991).

The goblet cells' function is the secretion of mucins and other products, including protease resistant peptides—like the trefoil peptide family, which protect epithelium from injury and promote repair through restitution of epithelial cells (Podolsky, 2000). Secretion of mucus occurs by exocytosis of secretory granules (Verdugo, 1991). Mucins have the ability to hydrate and form a viscous gel, producing a protective scaffold overlaying epithelial surfaces.

Constitutive or basal secretion occurs at low levels and is essentially unregulated and continuous. Stimulated secretion corresponds to regulated exocytosis of granules in response to extracellular stimuli such as hormones, neuropeptides and inflammatory mediators (Jackson, 2001; Laboisse et al., 1996). This pathway provides the ability to dramatically increase mucus secretion. The lumen of the intestinal tract inevitably contains numerous secretagogue irritants like gut bacteria (Deplancke and Gaskins, 2001). In the lung irritants such as dust and smoke are potent inducers of goblet cell secretion (Maestrelli et al., 2001). Besides stimulated exocytosis of stored mucin granules, prolonged exposure to secretagogue substances induces mucin gene expression and goblet cell hyperplasia (Ahlstedt and Enander, 1987; Maestrelli et al., 2001; Nadel, 2001). Epithelial cell differentiation in mucosal tissues has been studied to some detail in the gastrointestinal tact endoderm and the bronchial airways (Nadel, 2001; van Den Brink et al., 2001). In the intestinum, goblet cells differentiate from a multipotent stem cell, which gives rise to four epithelial cell types: enterocytes, goblet, enteroendocrine and Paneth cells (Yang et al., 2001). Recent genetic data provided evidence that the transcription factors Math1, Klf4 and Elf3 as well as the GTPase Rac1 are required for intestinal goblet cell differentiation in mice (Katz et al., 2002; Stappenbeck and Gordon, 2000; Yang et al., 2001). In airways, ligands of the epidermal growth factor receptor have been proposed to stimulate epithelial cell differentiation and mucin expression (Nadel, 2001).

As an alternative approach to identify genes involved in epithelial function we performed a genome wide screen for mutations influencing epithelial functions in nice, e.g. nutrient absorption by intestinal mucosa. Within this screen, a variant C3H mouse was identified which suffered from chronic diarrhea and impaired thriving. This mouse was fertile and the phenotype was transmitted to its offspring in a recessive fashion. This novel mouse variant is referred to as “MTZ” hereafter. Histological analysis demonstrated that the primary defect responsible for the observable phenotype in the novel C3H variant is a defective differentiation, particularly terminal differentiation, or function of goblet cells in its intestinal mucosa. The responsible mutation was identified by positional cloning and shown to result in an amino acid exchange within a known gene. This gene has been referred to as “anterior gradient 2” (Agr2) by the Mouse Genome Informatics database. Expression of the corresponding cDNA was described in murine intestinal tissues, specifically in intestinal goblet cells, by in situ hybridization (Komiya et al., 1999). The human orthologous gene, which encodes a protein with 91% amino acid identity, when compared to the mouse Agr2 gene, has been referred to as “Anterior gradient 2 homolog” (AGR2).

Human AGR2 (also termed BCMP7 and XAG-1) is a known protein. For example, WO 98/07749 discloses human growth factors, including a sequence identified as huXAG-1, which corresponds to human AGR2 and is suggested in that reference to be a growth factor and marker for colon cancer.

WO 99/53040 discloses a large number of sequences derived from an EST database, including sequences (identified as sequences ID 265 and 288), which correspond to AGR2.

WO 99/55858 again discloses a large number of sequences derived from an EST database, including sequences (identified as sequences ID 8 and 181), which correspond to AGR2 and are indicated as being more highly expressed in pancreas cancer tissue.

WO 00/53755 discloses a sequence (PRO 1030), which corresponds to AGR2. Using gene copy amplification, it is reported that the number of gene copies are increased in primary lung and colon tumor.

Sequences corresponding to AGR2 are also disclosed in WO 99/40189.

In US patent application 2002111303, AGR2 (referred to therein as BCMP 7) is predicted to be an extracellular protein with an N-terminal signal sequence and suggested to be a marker for breast cancer and prostate cancer.

Human AGR2 mRNA was shown to be expressed in trachea, lung, stomach, colon, prostate and small intestine (Thompson and Weigel, 1998).

cDNA sequences relating to human AGR2 are referred to in U.S. Pat. No. 6,312,922 (SEQ ID NOS:61 and 149).

The actual function of the AGR2 protein on the cellular level or on the level of the organism has not been described in mammals up to now. The only functional analysis of a protein homologue to AGR2 has been performed in Xenopus laevis, published by Aberger et al. (Aberger et al., 1998). The authors demonstrated that overexpression of XAG-2 induces both, ectopic cement gland differentiation and expression of anterior neural marker genes in Xenopus embryos. However, a Xenopus protein with the highest degree of amino acid identity, when compared to murine and human AGR2, is the protein CGS (EMBL/GenBank/DDBJ databases accession number AAL26844; TrEMBL entry Q90Y05), exhibiting 59% amino acid identity to murine Agr2 protein, and exhibiting 60% amino acid identity to human AGR2, respectively. The function of CGS, the putative AGR2 orthologue in Xenopus laevis, is not described yet.

In a detailed study we analyzed the RNA expression profile of the mouse Agr2 gene and the human AGR2 gene. The phenotype observed in the mouse model described herein demonstrates for the first time that Agr2 function is required for normal goblet cell function in a mammalian model organism.

Altered mucus production has been implicated in various diseases, e.g. asthma, chronic obstructive pulmonary disease (COPD), and cystic fibrosis, which are characterized by increased mucus production. Diseases like dry eye syndrome, gastric disease, peptic ulcer, and inflammatory bowel disease are characterized by decreased mucus production. Altered mucus production is also described in malignancies like colorectal cancer (Corfield et al., 2001; Einerhand et al., 2002; Fahy, 2001; Forstner, 1978; Jass and Walsh, 2001; Maestrelli et al., 2001; Melton, 2002; Puchelle et al., 2002; Schreiber et al., 2002; Slomiany and Slomiany, 2002; Velcich et al., 2002; Voynow, 2002; Watanabe, 2002). Therefore, great efforts are made in biomedical research to understand the mechanisms that are involved in epithelial cell differentiation, in the regulation of mucus production, in mucus secretion and in the maintenance of intact mucosal surfaces. Several strategies of modulating mucus production have been proposed (see the following patents and patent applications), e.g. by LTB4 antagonists (WO 02/55065), EGF receptor antagonists (WO 02/05842), polycationic peptides (U.S. Pat. No. 6,245,320), KGF (WO 94/23032) (Farrell et al., 2002) and KGF-2 (WO 99/41282). Several scientific reviews have been published recently covering epithelial cell differentiation in different tissue types containing mucus producing cells (Bhat, 2001; Brittan and Wright, 2002; Daniels et al., 2001; Emura, 2002; Foster et al., 2002; Otto, 2002). However, there has been no suggestion of an involvement of the AGR2 gene or its gene product in mucus production.

The invention described herein demonstrates for the first time that AGR2 is required for normal goblet cell function, in particular mucin secretion. The invention therefore opens novel opportunities for the diagnosis and treatment of said diseases involving malfunction of mucus producing tissues or any other condition, for which modulation of mucus production might have a therapeutic effect.

### SUMMARY OF THE INVENTION

In a first aspect, this invention provides a non-human animal useful as a model of goblet cell related disorders in humans, such as asthma, chronic obstructive pulmonary disease (COPD), cystic fibrosis, dry eye syndrome, gastric disease, peptic ulcer, inflammatory bowel disease, in particular Crohn's disease or ulcerative colitis, and malignancies like colorectal cancer.

In one embodiment, the animal of the invention carries a mutated AGR2 gene encoding an AGR2 protein with a modified amino acid sequence compared to the wild type sequence. In one embodiment, the AGR2 protein may have a modified amino acid sequence that causes a loss of function phenotype. Alternatively, the AGR2 may have a modified amino acid sequence that causes a gain of function phenotype.

The present invention also relates to methods using the animal model of the invention for the study of disorders associated with mutations in AGR2. In one embodiment, the invention provides methods of diagnosis for deficiencies or overproduction in AGR2, or the gene encoding it. In another embodiment, the invention provides a method for screening of preventive or therapeutic agents of disorders and symptoms associated to AGR2 mutations, using the animal model of the invention.

Furthermore, the present invention provides mutated AGR2 nucleic acids and polypeptides (also referred to as “muteins”) having modified sequences compared to the wild type sequences. These mutated nucleic acids and polypeptides may also be used in the diagnostic and therapeutic methods contemplated herein. In a specific embodiment, an AGR2 mutein carries an amino acid substitution at residue 137, as shown in SEQ ID NO:30.

Uses of the AGR2 muteins as modulators (whether agonists or antagonists) of endogenous AGR2 activity are also contemplated. Consequently, pharmaceutical compositions comprising the AGR2 muteins of this invention are contemplated further comprising a pharmaceutically acceptable carrier. Specifically we contemplate use of the AGR2 muteins of the present invention, the polynucleotide encoding them and vectors bearing the polynucleotides for the prevention, treatment or amelioration of a medical condition in a mammalian subject, particularly a human subject, and in particular their use for the development of a measure for the prevention, treatment or amelioration of any medical conditions characterized by goblet cell abnormalities or mucus production.

One embodiment of the invention is related to a method for modulating the expression of a target gene in a eukaryotic cell when the target gene is regulated by the AGR2 protein. The method involves the step of modulating the activity of AGR2, i.e., of the wild type AGR2 or the AGR2 mutein. While the method may be used on single cells, it is preferable to apply the method to a eukaryotic cell within a multicellular organism, for example, in a mammal such as a human, horse, dog, cat, sheep, rat, or a mouse, but also in other vertebrates, such as amphibians, e.g., in Xenopus leavis. The eukaryotic cell within the above multicellular organisms may be a cell that expresses AGR2, in particular a goblet cell or a mucus secreting cell of, e.g., the Brunner's gland or the submucosal glands of the trachea.

Another embodiment of the invention is related to a method for modulating the expression, in a cell of a mammal, of a target gene whose transcription is regulated by AGR2 protein. In the method, the activity of AGR2 is modulated, i.e., the activity of the wild type or mutein AGR2, and the modulated AGR2 will, in turn, modulate the expression of the target gene. The method will work on all animals, for example in mammals such as human, horse, dog, cat, sheep, rat, or mouse, but also in other vertebrates, such as amphibians, e.g., Xenopus leavis. The method is particularly useful in cells which express AGR2, such as goblet cells or mucus secreting cells of, e.g., the Brunner's gland or the submucosal glands of the trachea.

The activity of AGR2, i.e., of the wild type or mutein AGR2, may be modulated in a number of ways, such as, for example, altering the state of posttranslational modification. For example, if the target gene is responsive to a phosphorylated AGR2, the phosphorylation state of AGR2 protein may be increased to increase the activity of the target gene. Conversely, if it is desired to reduce the activity of the gene, the phosphorylation state of AGR2 may be decreased.

As another example, if the target gene is responsive to a dephosphorylated state of AGR2, the phosphorylation state of AGR2 is decreased to increase the activity of the target gene. In this case, the phosphorylation state of AGR2 may be increased to reduce the activity of the target gene.

The modulation may involve both an increase of AGR2 activity, i.e., of the wild type or mutein AGR2, or a decrease of AGR2 activity, i.e., of the wild type or mutein AGR2. Any method that can increase or decrease AGR2 activity may be used. For example, AGR2 may be decreased by contacting an AGR2 expression inhibitor with an AGR2 mRNA to prevent protein translation or promote mRNA decay. The AGR2 expression inhibitor may be a biomolecule such as a nucleic acid. For example, the nucleic acid may be an antisense nucleic acid (DNA, RNA, PNA or other synthetic nucleic acid analogs), an siRNA molecule, or an aptamer. The nucleic acid may be a ribozyme specific for AGR2 mRNA. In all cases where a nucleic acid is used, the nucleic acid may be designed to differentiate between a nucleic acid encoding a mutated protein from a wild type nucleic acid. For example, the ribozymes and antisense nucleic acids may be designed to hybridize in a sequence specific manner to the sequence encoding the mutated AGR2 but not to the sequence encoding the wild type AGR2.

Alternatively, the ribozymes and antisense nucleic acids may be designed to hybridize in a sequence specific manner to the sequence encoding the wild type AGR2 but not to the sequence encoding the mutated AGR2.

As a further example, a ribozyme discussed above may be comprised of a hybridizing region and a catalytic region. A ribozyme designed to affect AGR2 expression will, naturally, contain a hybridizing region that is capable of hybridizing to at least part of a AGR2 mRNA sequence. Further, the ribozyme would contain a catalytic domain capable of cleaving the AGR2 mRNA sequence to reduce or inhibit AGRn gene expression. The hybridizing region may be constructed to hybridize only to a sequence encoding a mutated AGR2 and not to a sequence encoding wild type AGR2. Alternatively, the hybridizing region may be constructed to hybridize only to a sequence encoding a wild type AGR2 and not to a sequence encoding mutant AGR2, i.e., the hybridization region does not comprise a part of the AGR2 mutein sequence encompassing the mutation. Conversely, the hybridizing region may be constructed to hybridize to all sequences encoding AGR2 regardless of whether the protein is wild type or mutant.

In another embodiment, the biomolecule, discussed above, may be a protein. The protein may be an antibody, a fragment of an antibody, or an anticalin. These antibody and antibody fragments may show specificity in binding the AGR2 protein, i.e., the wild type or mutein AGR2. While antibodies and antibody fragments with high specificity are preferred, lower specificity antibodies and fragments are also contemplated by this invention. A lower specificity antibody or fragment may be useful, for example, if the antibody does not interfere with other cellular functions.

Preferably, the specificity of the antibodies and antibody fragments is sufficient so that they do not bind any other protein in the cell. High specificity may be achieved by using monoclonal antibodies. Methods for making monoclonal antibody are well known. Other methods for making polyclonal antibodies, such as, for example, by injection into animals are also known. High specificity polyclonal antibodies may be produced, for example, by using a column bound with proteins from a cell not expressing AGR2 (i.e., column chromatography) of polyclonal antibodies. Such a column would remove nonspecific antibodies. Other techniques for purifying antibodies are known in the art.

Another embodiment of the invention is related to a mutant AGR2 polypeptide, comprising, e.g., an amino acid substitution at the position corresponding to residue 137 of SEQ ID NO:2. The polypeptide may contain at least 6 amino acids, preferably at least 7 amino acids, more preferably at least 8 amino acids, even more preferably at least 9 amino acids and most preferably at least 10 amino acids. Longer peptides, such as, for example, the complete AGR2 protein containing an amino acid substitution at position 137 are, of course, contemplated because the complete protein is longer than the limit of at least 6, 7, 8, 9 or 10 amino acids stated above.

The amino acid substitution is the substitution of a codon encoding valin at position 137 to a codon encoding a non-valin substitution. The genetic code is known so the types of substitution claimed are known to one of skill in the art. One example of substitution may be one in which valin is substituted by an acidic amino acid such as glutamic acid or aspartic acid. Another example of substitution may be one in which valin is substituted by a glycine or proline. Another example of substitution may be one in which valin is substituted by a basic amino acid (histidin, arginin or lysin), aliphatic hydroxyl side chain amino acid (serine, threonine), aromatic side chain amino acid (phenylalanine, tyrosine, tryptophan), amide side chain amino acid (asparagine, glutamine), sulfur containing side chain amino acid (cysteine, methionine) or aliphatic side chain amino acid (alanine, leucine or isoleucine).

One embodiment of the invention is related to a nucleic acid segment that encodes a polypeptide fragment of AGR2 where the polypeptide fragment comprises an amino acid substitution corresponding to residue 137 of the full length AGR2. The amino acid substitution may be the replacement of the codon encoding residue 137 with any codon that do not encode valin. A codon that does not encode valin may be, for example, a codon that encode Phe (TTT, TTC); Leu (TTA, TTG, CTT, CTC, CTA, CTG); Ile (ATT, ATC, ATA); Met (ATG); Ser (TCT, TCC, TCA, TCG), Pro (CCT, CCC, CCA, CCG); Thr (ACT, ACC, ACA, ACG), Ala (GCT, GCC, GCA, GCG); Tyr (TAT, TAC); His (CAT, CAC), Asp (GAT, GAC); Gln (CAA, CAG); Asn (AAT, AAC); Lys (AAA, AAG); Glu (GAA, GAG); Cys (TGT, TGC); Trp (TGG); Arg (CGT, CGC, CGA, CGG, AGA, AGG); Ser (AGT, AGC); or Gly (GGT, GGC, GGA, GGG). Of all the substitutions stated above, a nucleic acid that encodes a substitution of valin to glutamic acid (GAA, GAG) at codon 137, as shown in SEQ ID No:2 is most preferred.

The nucleic acid of the invention may be part of a recombinantly generated episomal element. Episomal elements may be, for example, a plasmid, cosmid, bacterial phage nucleic acid, or a viral nucleic acid. The recombinantly generated nucleic acid may be a part of a genome, such as a bacteriophage genome, a bacteria genome, or virus genome. Virus genomes may be a DNA viral genome, or an RNA viral genome (both + strand virus or − strand virus).

In another embodiment, the invention is related to vectors comprising a nucleic acid segment that encodes a polypeptide fragment of AGR2 where the polypeptide fragment comprises an amino acid substitution corresponding to residue 137 of the full length AGR2. The vector may be an expression vector, a mutagenesis vector, an integration vector or a mutation vector. Expression vectors are well known in the art and include plasmid vectors, cosmid vectors, phage vectors, phagemid vectors, viral vectors, retroviral vectors, and the like.

The invention also contemplates a host cell transfected with one of the vectors and nucleic acids described above. A host cell may be, for example, a eukaryotic cell or a prokaryotic cell. A host cell transformed with a nucleic acid that is not a vector may be, for example, a cell transformed with antisense DNA or a ribozyme.

Another embodiment of the invention is related to a method of producing a mutant AGR2 protein. In the method, a host cell transfected with a nucleic acid that encodes a polypeptide fragment of AGR2 where the polypeptide fragment comprises an amino acid substitution corresponding to residue 137 of the full length AGR2 is cultured such that the nucleic acid is expressed. It should be noted that an expression vector may be desirable but is not required. For example, in transient expression, vector sequences are not required for expression. The cultured cells are then harvested and the mutant AGR2 protein is purified from the cells. While purification to homogeneity may be desirable, it is not necessary. Purification may involve merely making a lysate from bacteria that expressed AGR2. In this example, the AGR2 protein is purified because it is no longer associated with the proteins it was naturally associated with (i.e., 10 eukaryotic proteins). As another example, a mouse Agr2 protein expressed in a human cell is also purified because it is no longer associated with the proteins (mouse proteins) that it is naturally associated.

Another embodiment of the invention is related to a composition for inducing an altered condition in a patient. The composition may comprise a mutant AGR2 polypeptide containing a substitution mutation that corresponds to residue 137 or any other AGR2 mutein described herein. Examples of wild type AGR2 proteins are shown in SEQ ID NO:3, or SEQ ID NO:4. Thus, a polypeptide with a substitution mutation in codon 137 of SEQ ID NO:3 or SEQ ID NO:4 may be an ingredient in the composition. The substitution mutation may be the substitution of valin at position 137 with a non valin amino acid. The composition may also comprise a wild type AGR2 protein, e.g., a protein according to SEQ ID NO:4. In addition, the composition may contain a pharmaceutically acceptable carrier.

Another embodiment of the invention is related to a method of selectively inhibiting the expression, in a eukaryotic cell of a gene whose transcription is negatively or positively regulated by AGR2. The eukaryotic cell is preferably a mammalian cell, preferably a cell derived from a human, horse, dog, cat, sheep, rat, or a mouse, but also derived from other vertebrates, such as amphibians, e.g., from Xenopus leavis. The method is also related to cells within the aforementioned animals, preferably within a human. The eukaryotic cell may be a cell that itself expresses AGR2, in particular a goblet cell or a mucus secreting cell of, e.g., the Brunner's gland or the submucosa of the trachea.

Another embodiment of the invention is related to a method for expressing an AGR2 protein with altered activity. In the method, a host cell with an episomal element that comprises a cDNA which encodes AGR2 protein with, e.g., a substitution mutation, wherein the mutation is a substitution of valin at position 137 with an amino acid that is not valin is provided. Then the host cell is cultured such that the mutant AGR2 protein is expressed.

Another embodiment of the invention is related to an antisense nucleic acid molecule of a length sufficient to inhibit the expression of an AGR2 protein, i.e., a wild type AGR2 protein or an AGR2 mutein. An antisense nucleic acid molecule sufficient to inhibit total cellular AGR2 protein biological activity is also contemplated. The antisense nucleic acid molecule is complementary to a mammalian AGR2 nucleic acid sequence such as human AGR2 sequence, mouse Agr2 sequence, or rat AGR2 sequence. The biological activity to be inhibited may be goblet cell function, e.g., mucus production, or the proliferation of mucus secreting cells of, e.g., the glandular epithelium of the Brunner's gland. The activity may be inhibited by at least 5%, 10%, 15%, 20%, 25%, 50%, 75% or 100%. The antisense nucleic acid may be at least 15 nucleotides in length.

Another embodiment of the invention is related to a ribozyme. The ribozyme comprises a hybridizing region and a catalytic region. The hybridizing region is capable of hybridizing to at least part of a target mRNA sequence transcribed from a genomic AGR2 sequence and the catalytic domain is capable of cleaving the target mRNA sequence to reduce or inhibit AGR2 function, i.e., the function of a wild type AGR2 protein or an AGR2 mutein.

Another embodiment of the invention is related to an siRNA molecule. The siRNA molecule is designed in a way to efficiently inhibit the Agr2 gene expression, i.e., the gene expression of the wild type AGR2 or the AGR2 mutein, by gene silencing.

A further embodiment of the invention is related to an aptamer. The aptamer is designed in a way to efficiently bind AGR2, i.e., the wild type AGR2 or the AGR2 mutein. Preferably, the specificity of the aptamers is sufficient so that they do not, or substantially do not, bind to any other protein in the cell.

Another embodiment of the invention is related to a pharmaceutical composition, which comprises a nucleic acid molecule that inhibits or otherwise reduces AGR2 mediated function, i.e., wild type AGR2 or AGR2 mutein function. The nucleic acid is at least about ten nucleotides in length and hybridizes to an AGR2 mRNA molecule or forms a heteroduplex with a AGR2 mRNA molecule. The nucleic acid molecule may be an antisense molecule or an siRNA molecule. The pharmaceutical composition, in addition to the nucleic acid described, further comprises one or more pharmaceutically acceptable carriers.

Another embodiment of the invention is related to a transgenic non-human mammal all of whose germ cells and somatic cells contain a mutated AGR2 gene, which was introduced into the mammal, or one of its ancestors, at an embryonic stage. The transgene—a mutated AGR2 gene—encodes, e.g., an amino acid substitution mutation at the position corresponding to amino acid 137 of the AGR2 protein.

The mutated AGR2 protein of the transgenic mammal above may be derived from a wild type AGR2 protein sequence. Wild type AGR2 proteins are listed in SEQ ID NO:3 or SEQ ID NO:4. A mutated version of the AGR2 protein would contain the sequence of SEQ ID NO:3 or SEQ ID NO:4 but with a substitution mutation at, e.g., amino acid 137. The substitution mutation may be the substitution of valin at position 137 with a non-valin amino acid. The transgenic mammal may further contain a knockout wild type AGR2 gene. Furthermore, the knockout wild type AGR2 gene may be homozygous such that the transgenic animal contains no wild type AGR2. In this case, the only AGR2 gene in the transgenic animal is the mutated AGR2 gene. Naturally, since the only AGR2 gene is the mutated one, the only AGR2 protein in the transgenic animal is the mutated AGR2 protein. There are multiple methods of constructing an animal with knockout endogenous AGR2 and a functional mutant AGR2. One method is to knockout both endogenous AGR2 genes by homologous recombination. An easier method may be to knockout one of the endogenous AGR2 gene and breed this knockout AGR2 locus to homozygosity. The introduction of a mutant AGR2 gene may be part of the knockout construction. That is, the genetic construct designed to target the endogenous AGR2 gene may itself contain a mutant AGR2 gene. Thus, the gene knockout and the introduction of a mutant AGR2 gene may be performed concomitantly. Alternatively, a knockout animal line (homozygous or heterozygous) may be used to produce transgenic animals using a mutated AGR2 DNA construct Finally, a knockout AGR2 animal line may be crossed with a transgenic animal carrying a mutant AGR2 gene. Animals homozygous for AGR2 knockout and for carriers of a mutant AGR2 can be made using standard genetic techniques.

In the cases where the mutant AGR2 gene construct is used to produce a transgenic animal, the gene construct may further comprise a promoter sequence different from the promoter sequence controlling the transcription of the endogenous AGR2 coding sequence. Thus, mutant AGR2 may be expressed in any desired tissue depending on the choice of promoter sequence. Further, the promoter sequence may be from an inducible promoter. While the transgenic non-human mammals of this invention may be any mammal, one preferred animal is a rodent such as a rat or a mouse.

Another embodiment is related to the use of the nucleic acids of the invention for in vivo delivery and expression. This approach has also been called gene therapy. It should be noted that to be useful, gene therapy does not need to be completely efficacious. A method of gene therapy that can alleviate a symptom of a mammalian disorder is envisioned by the instant disclosure. Gene therapy is known in the art. This term has been used to describe a wide variety of methods using recombinant biotechnology techniques to deliver a variety of different materials to a cell. Such methods include, for example, the delivery of a gene, antisense RNA, an siRNA molecule, an aptamer, a cytotoxic agent, etc., by a vector to a mammalian cell, preferably a human cell either in vivo or ex vivo. Most work has focused on the use of viral vectors to transform these cells. This focus has resulted from the ability of some viruses, to infect cells and have their genetic material integrated into the host cell with high efficiency. Viruses useful for this approach include retroviruses, adenoviruses, pox viruses (including vaccinia), herpes virus, etc. In addition, various non-viral vectors such as ligand-DNA-conjugates have been used. Transient expression of transgenes has been developed also by the use of non-integrative viral vectors with low replicative efficiency.

Other embodiments of the invention are related to the use of the nucleic acids and proteins as described herein to alter or modulate, in a cell of a mammal, the expression or activity of AGR2, i.e., the AGR2 wild type protein or mutein; or to their use to alter or modulate the expression of a target gene whose transcription is directly or indirectly regulated by AGR2 protein.

The use described above, when applied to an animal such as a mammal (e.g., a human) have significant medicinal value. Thus, another embodiment of the invention is related to the use of the proteins and nucleic acids as described herein as a medicament. The medical composition may be used to prevent, to ameliorate, or to treat a disease such as asthma, chronic obstructive pulmonary disease (COPD), cystic fibrosis, dry eye syndrome, gastric disease, peptic ulcer, inflammatory bowel disease and malignancies like colorectal cancer. The medical condition or disease may optionally furthermore be associated with an increased proliferation of the glandular epithelium of the Brunner's gland.

The proteins (i.e., all proteins described including AGR2 wild type or mutein, antibodies and other proteins), chemical molecules, including small molecules, e.g., small molecule agonists or small molecule antagonists, and nucleic acids of the invention may be applied to a patient using well known delivery methods as described infra. The medicament may be used for the modulation of goblet cell function. The compositions and medicament of the invention may be used to alter the biological activity of AGR2, i.e., the AGR2 wild type protein or mutein.

Further embodiments of the invention relate to the use of the vectors, episomal elements and/or host cells as described herein for prevention, amelioration, or treatment of those diseases associated with goblet cell activity or deficiency, such as asthma, chronic obstructive pulmonary disease (COPD), cystic fibrosis, dry eye syndrome, gastric disease, peptic ulcer, inflammatory bowel disease and malignancies like colorectal cancer and the use of the non-human animal model of the invention for the dissection of the molecular mechanisms physiological processes within which AGR2 is active, or which are influenced by AGR2.

Further embodiments include the use of the non-human animal model of the invention for the identification of gene and protein diagnostic markers for diseases, or for the identification and testing of compounds useful in the prevention, amelioration, or treatment of those diseases associated with AGR2 activity or deficiency, as described herein.

The above embodiments and yet further embodiments of the present invention will be explained in more detail below.

### DESCRIPTION OF THE FIGURES

FIG. 1 depicts the synthetic chromosomal regions of mouse and human bearing the AGR2 genes of both species (FIG. 1A), and a comparison of the exon-intron structure (FIG. 1B) of murine and human AGR2. Only coding exons are coloured in grey. Exon sizes are indicated by the number of basepairs either top of an exon (if coding exon) or below an exon (if non-coding exon). Intron sizes are depicted in length by basepairs.

FIG. 2 depicts an alignment of the murine and human wild type AGR2 protein sequences, indicating the amino acid residues identity between the two proteins. The position of the mutation is highlighted in grey.

FIG. 3 depicts a chart diagramming the F3-production (FIG. 3A) and the outcross breeding schemes (FIG. 3B) used to map the mutation, associated with the observed phenotypic abnormalities, to mouse chromosome 12. Legend: thin parallel lines represent the two alleles of the genome, crossed thin lines represent mutation events; thick lines represent the wild type of a different mouse strain used for outcrossing.
m WT indicates a male wild type;
f WT indicates a female wild type;
DB1 indicates a dominant breeding 1;
RF1 indicates a recessive F1×F1;
RBS indicates a recessive brother-sister;
ROC indicates a recessive out-cross;
RIC indicates a recessive inter-cross.
Abbreviations in miniscules indicate the animal involved in each breeding stage, their names indicating the stages from which they were generated.

FIG. 4 depicts final data from genome wide SNP analysis on affected F5 MTZ mice leading to the assignment of the mutation to proximal chromosome 12, as performed by Pyrosequencing Technology.

FIG. 5 depicts a haplotype scheme of informative MTZ mice with chromosomal breakpoints defining the location of the mutation at chromosome 12 between marker Idb2 and marker D12Mit64. The symbols “c”, “hz” and “b”, respectively, indicate C3H (c) mice, heterozygous (hz) mice, and c57B16 (b) mice, respectively.

FIG. 6 depicts data from a reverse transcribed polymerase chain reaction (RT-PCR) analysis, examining murine AGR2 mRNA expression at murine tissue cDNAs. The 349 bp band represents the PCR product specific for murine AGR2.

FIG. 7 depicts data from a reverse transcribed polymerase chain reaction (RT-PCR) analysis, examining human AGR2 mRNA expression at human tissue cDNAs. The 170 bp band represents the PCR product specific for human AGR2.

FIG. 8 depicts Northern blots hybridized with a human AGR2 probe.

FIG. 9 depicts a table listing genotypes and phenotypes of mice descending from the MTZ mouse originally identified in the genome wide mutagenesis screen. Mice carrying the missense mutation of the Agr2 gene on both alleles are marked as “mut”, whereas those carrying one mutated and one wild type allele are marked as “het”. Mice carrying two wild type alleles at the Agr2 locus are marked as “W”. All mice carrying the missense mutation of the Agr2 gene on both alleles display the MTZ phenotype, i.e. chronic diarrhea and reduced thriving, whereas all other mice were phenotypically normal.

FIG. 10 depicts cross sections of the colon walls of a wild type mouse in C3H genetic background. The samples were formalin fixed and stained with anti-TFF3 (trefoil peptide 3) antibody and anti-murin Agr2 antiserum, respectively—indicating TFF3 and Agr2 expression in goblet cells.

FIG. 11 depicts a cross section of the colon walls of an MTZ mouse in the C3H genetic background, and a respective wild type mouse used as a control. The samples were formalin fixed and stained with H/E (hematoxilin/eosin). In the wild type animal, goblet cells are characterized by their high content of vesicles storing pre-mucins and other components of mucus, which appear as light spherical droplets in the present staining. These droplets are almost absent in the colon epithelium of the MTZ animal.

FIG. 12 depicts a cross section of the colon walls of an MTZ mouse in the C3H genetic background. The samples were formalin fixed and stained with H/E (hematoxilin/eosin). The colon wall of the MTZ animal contains infiltrating inflammatory immune cells in the mucosal epithelium and submucosa, which are identifiable by their small size and the dark staining spherical nucleus (marked by an asterisk. In addition, microerosion of colonic mucosa is detected and marked by an arrow.

FIG. 13 depicts a cross section of the colon walls of an MTZ mouse in the C3H genetic background and a respective wild type mouse used as a control. The samples were formalin fixed and stained with the fluorescent labeled lectins wheat germ agglutinin (WGA), and with a Dolichos biflorus agglutinin (DBA). In the wild type animal, highly glycosylated mucins are identifiable by their light staining, which concentrates in spherical droplets stored by goblet cells. In contrast, these light staining droplets are almost absent in the colon epithelium of the MTZ animal.

FIG. 14 depicts a cross section of the duodenal wall of an MTZ mouse in the C3H background and a respective wild type mouse as a control. The samples were formalin fixed and stained with H/E (hematoxilin/eosin). In the wild type animal, a normal Brunner's gland as well as normal duodenal epithelium are detected. In the MTZ animal the Brunner's gland is dilated and the duodenal epithelium is proliferating. In the MTZ animal the Brunner's gland is dilated and the duodenal epithelium is proliferating. A Brunner's gland is indicated by an asterisk, a duodenal epithelium is indicated by an arrow.

FIG. 15A depicts the results when applying the amino acids 1 to 30 from mouse Agr2 to the publicly available program “SignalP V1.1” (Nielsen et al., 1997). The program predicts an N-terminal signal sequence encoded by the amino acids 1 to 20 and a cleavage site between amino acid 20 and 21 with a high probability.

FIG. 15B depicts the results when applying the amino acids 1 to 30 from human AGR2 to the publicly available program “SignalP V1.1” (Nielsen et al., 1997). The program predicts a N-terminal signal sequence encoded by the amino acids 1 to 20 and a cleavage site between amino acid 20 and 21 with a high probability.

FIG. 16 depicts the comparison of the amino acid sequences of mouse, human, and rat Agr2 proteins. Amino acid identity of 91%, and amino acid similarity of 95% indicate evolutionary highly conserved amino acid residues. The conserved amino acids (i.e., identical or similar) are listed in accompanying Table 1.

FIG. 17 depicts the comparison of the amino acid sequences of mouse, human, rat, and Xenopus laevis Agr2 proteins. Amino acid identity of 67%, and amino acid similarity of 82% indicate evolutionary highly conserved amino acid residues. The conserved amino acids (i.e., identical or similar) are listed in accompanying Table 2.

FIG. 18 depicts the comparison of the amino acid sequences of mouse, human, rat, Xenopus laevis, and C. elegans Agr2 proteins. Amino acid identity of 32%, and amino acid similarity of 46% indicate evolutionary highly conserved amino acid residues. The conserved amino acids (i.e., identical or similar) are listed in accompanying Table 3.

FIG. 19 depicts data from quantitative mRNA detection by PCR-Light Cycler technology on freshly prepared colon cDNA of MTZ and wild type control newborns. Elevated amount of Agr2 transcript is accompanied by reduced amounts of muc2 (mucin 2) and TFF3 transcript Both genes, Muc2 and TFF3 encode proteins that comprise the major components of mucus. Same data have been established in assays with colon cDNA of adult MTZ and wild type control mice. Regulation of mRNA was determined as x fold change relative to the transcript amount of internal standard gene ALAS (aminolevulinic acid synthase 1).

FIG. 20 depicts Western blot data indicating secretion of AGR2 protein into the supernatant conditioned from colon cancer cell lines.

### DETAILED DESCRIPTION OF THE INVENTION

The various aspects and utilities of the present invention will be apparent from the following detailed description.

The goblet cells referred to herein are cells, which are specialized with respect to mucus secretion via granules, in particular in the gastrointestinal tract (GI) (examples in this regard are goblet cells of the esophagus, of the stomach surface, of the pyloric glands, and of the intestinal epithelium), or in the respiratory tract (examples in this regard are goblet cells of the nose epithelium, of the trachea, of the bronchius, and of the submucosal glands of the trachea).

The term “differentiation” as used herein in connection with goblet cells refers to all steps of cellular differentiation of a goblet cell from early differentiation to late differentiation and to terminal differentiation, i.e., to the mature mucus secreting goblet cell. Thus, terminal differentiation of goblet cells means the last differentiation step to the mature goblet cell.

The term “mucus secreting cell” as used herein refers to cells which are specialized to mucus secretion without prior storage of the mucus in granules, e.g., the mucus secreting cells of the Brunner's gland.

### Animal Model and its Uses

The present invention provides, for example, a non-human vertebrate animal expressing an AGR2 protein which is modified compared to the amino acid sequence of the wild type protein at amino acid position 137. The animal may be a mammalian animal, preferably a rodent, in particular from a genus such as Mus (e.g. mice), Rattus (e.g. rats), Oryctologus (e.g. rabbits) and Mesocricetus (e.g. hamsters). In a particularly preferred embodiment the animal is a mouse. However, dogs, cats, sheep, and horses are likewise suitable in connection with the invention. The same applies to vertebrates such as amphibians, in particular Xenopus laevis.

The term “modified” as used herein in connection with the AGR2 protein and nucleic acids relating thereto refers to an alteration compared to the wild type AGR2, e.g., the wild type AGR2 proteins according to SEQ ID NO:3 or SEQ ID NO:4.

The term “phenotype” as used herein refers to a collection of morphological, physiological, behavioral and/or biochemical traits possessed by a cell or organism that result from the interaction of the genotype and the environment. Thus, the non-human vertebrate animal of the present invention displays readily observable abnormalities compared to the wild type animal. In a preferred embodiment the animal of the invention shows at least 1, preferably at least 2, and most preferably at least 4 abnormal phenotypical features, preferably selected from all of the above categories.

More generally, the non-human vertebrate animal according to the present invention comprises in the genome of at least some or all of its cells an allele of a gene encoding a protein having at least 65%, at least 70%, at least 75%, at least 80%, at least 85%, at least 90%, at least 95%, at least 98%, or at least 99% amino acid identity compared to the mouse Agr2 or the human AGR2 protein according to SEQ ID NO:3 and SEQ ID NO:4, respectively.

The following definitions apply to any reference to nucleic acid or amino acid sequence identity throughout the present specification. The term “sequence identity” refers to the degree to which two polynucleotide or polypeptide sequences are identical on a residue-by-residue basis over a particular region of comparison. The phrases “percent amino acid identity” or “% amino acid identity” refer to the percentage of sequence identity found in a comparison of two or more amino acid or nucleic acid sequences. Percent identity can be readily determined electronically, e.g., by using the MEGALIGN program (DNASTAR, Inc., Madison Wis.). The MEGALIGN program can create alignments between two or more sequences according to different methods, one of them being the clustal method. See, e.g., Higgins and Sharp (Higgins and Sharp, 1988). The clustal algorithm groups sequences into clusters by examining the distances between all pairs. The clusters are aligned pairwise and then in groups. The percentage similarity between two amino acid sequences, e.g., sequence A and sequence B, is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity.

A particularly preferred method of determining amino acid identity between two protein sequences for the purposes of the present invention is using the “Blast 2 sequences” (bl2seq) algorithm described by Tatusova et al. (Tatiana A. Tatusova, Thomas L. Madden (1999), “Blast 2 sequences—a new tool for comparing protein and nucleotide sequences”, FEMS Microbiol Lett. 174:247-250). This method produces an alignment of two given sequences using the “BLAST” engine. On-line access of “blasting two sequences” can be gained via the NCBI server at http://www.ncbi.nlm.nih.gov/blast/bl2seq/bl2.html. The stand-alone executable for blasting two sequences (bl2seq) can be retrieved from the NCBI ftp site (ftp://ftp.ncbi.nihgov/blast/executables). Preferrably, the settings of the program blastp used to determine the number and percentage of identical or similar amino acids between two proteins were the following:

| | |
|---|---|
| Program: | blastp |
| Matrix: | BLOSUM62 |
| Open gap penalty: | 11 |
| Extension gap penalty: | 1 |
| Gap x_dropoff: | 50 |
| Expect: | 10.0 |
| Word size: | 3 |
| Low-complexity filter: | on |

For the purposes of the present specification, a reference to percent amino acid sequence identity means in a preferred embodiment percent identity as determined in accordance with the blastp program using the above settings.

The protein mentioned above may be, for example, the corresponding orthologue of the mouse Agr2 or the human AGR2 protein according to SEQ ID NO:3 and SEQ ID NO:4 with respect to the animal. It may also be a variant of the mouse Agr2 or the human AGR2 protein according to SEQ ID NO:3 and SEQ ID NO:4, or of said orthologue, allelic or otherwise, wherein certain amino acids or partial amino acid sequences have been replaced, added, or deleted.

In a preferred embodiment, the genome of the cells of the animal comprising said allele does not additionally comprise more than one functional allele representing a wild type AGR2 gene, for example the corresponding wild type orthologue with respect to the animal, or a wild type AGR2 gene that is heterologous with respect to the genomic DNA of the cells. It is particularly preferred that the genome of the above cells does not additionally comprise any functional allele representing a wild type AGR2 gene (i.e., no functional allele of the corresponding wild type orthologue, or of a heterologous wild type AGR2 gene).

The above-mentioned mutated allele comprised in the genome of the cells of the non-human vertebrate animal comprises a mutation which, if present in the genome of all or essentially all cells of said animal in a homozygous manner, in particular in the animal's goblet cells, results in a phenotype associated with an alteration in goblet cell function compared to the corresponding wild-type animal. It will be appreciated that this mutation may reside in either the coding or the non-coding region of the allele.

The above-mentioned phenotypes may be characterized by an alteration in goblet cell differentiation, particularly terminal differentiation, or an alteration in goblet cell mucus production or secretion. They may also be characterized by an alteration in mucus composition, e.g., in respect of the levels of typical mucus constituents, e.g., mucin2 (muc2) or trefoil peptides. Such phenotypes may also be characterized by any combination of these phenomena.

A typical phenotype of a non-human vertebrate animal in this regard is one characterized by a reduction in pre-mucin storing granules in the goblet cells, an altered mucus secretion, secondary inflammatory infiltrations in the intestinal mucosal epithelium and submucosa. The phenotype of the non-human vertebrate animal as described herein may optionally be furthermore associated with an increased proliferation of the glandular epithelium of the Brunner's gland.

The phenotype of the non-human vertebrate animal according to the present invention may further be characterized by reduced transcription levels of the late differentiation markers Muc-2 and TFF3 in goblet cells.

Furthermore, a typical phenotype of a non-human vertebrate animal according to the present invention is one wherein the alteration results in diarrhea, or diarrhea and a thriving deficit.

In another non-human vertebrate animal according to the present invention the mutated allele contains a mutation corresponding to a mutation in the mouse Agr2 protein or the human AGR2 protein according to SEQ ID NO:3 and SEQ ID NO:4, respectively, which leads to an altered biological activity of the mutated protein when compared to the corresponding wild type mouse Agr2 protein or human AGR2 protein in an in vitro assay.

The term “corresponds to” as used in this regard and throughout the present specification means that the mutated allele reflects the mutation in the mouse Agr2 protein or the human AGR2 protein according to SEQ ID NO:3 and SEQ ID NO:4 on the amino acid level. Where the sequences of the allele flanking the mutation do not encode amino acids identical to those at the corresponding positions in the amino acid sequences of the mouse Agr2 or the human AGR2 protein defined above, the skilled artisan will be readily able to align the amino acid sequences encoded by the flanking sequences with the corresponding amino acids of the mouse Agr2 or the human AGR2 protein, preferably by using the above-mentioned method of determining amino acid sequence identity, and determine whether a mutation in the mouse Agr2 protein or the human AGR2 protein of the kind mentioned above is reflected by the amino acid sequence encoded by said allele. In case of an amino acid substitution or insertion, the mutation is preferably reflected by the amino acid sequence encoded by the allele in such a way that an identical amino acid or amino acid sequence is found at the corresponding position of the protein encoded by the allele. In case of an amino acid deletion, the mutation is preferably reflected by the amino acid sequence encoded by the allele in such a way that an identical or corresponding amino acid or amino acid sequence is deleted at the corresponding position of the protein encoded by the allele.

The term “altered biological activity in an in vitro assay” as used above in connection with the reference to the in vitro assay and throughout the present specification refers either to an increased or a decreased biological activity. The increase in biological activity is preferably an at least 10%, 20%, 30%, 40%, 50%, 70%, 80%, 90%, or a 100% or an even higher increase as compared to the wild type mouse Agr2 protein or human AGR2 protein according to SEQ ID NO:3 and SEQ ID NO:4, respectively. Likewise, the decrease in biological activity is preferably an at least 10%, 20%, 30%, 40%, 50%, 70%, 80%, or 90% decrease, or an even complete abolishment of biological activity as compared to the wild type mouse Agr2 protein or human AGR2 protein according to SEQ ID NO:3 and SEQ ID NO:4, respectively. Since the increase or decrease in biological activity are determined by comparing mouse Agr2 or human AGR2 muteins carrying the corresponding mutation to wild type mouse Agr2 or human AGR2 protein in the same assay, preferably side-by-side and under the same assay conditions, therefore resulting in relative values, it will be appreciated that the skilled person will be readily able to determine the above percentages of alteration in biological activity in the in vitro assays contemplated in connection with the present invention.

Monitoring colon cell proliferation is one suitable assay to determine altered biological activity of a AGR2 mutein according to the present invention compared to wild type mouse Agr2 protein or human AGR2 protein. One assay preferred in this regard is described herein in Example 20. In such a preferred assay, the incorporation of a label added to the culture medium into the cellular DNA of the cultured colon cells is monitored. The cultured cells are preferably mammalian colon cancer cell lines. Particularly preferred are the mammalian colon cancer cell lines LS174T or HT29. Cells are transfected with a wild type AGR2 expression vector (e.g., a vector expressing mouse Agr2 protein or human AGR2 protein according to SEQ ID NO:3 and SEQ ID NO:4, respectively), or with an expression vector expressing the AGR2 mutein of interest (i.e., expressing any of the novel AGR2 proteins or protein fragments described and claimed herein). Alternatively, AGR2 wild type protein (again preferably mouse Agr2 protein or human AGR2 protein according to SEQ ID NO:3 and SEQ ID NO:4, respectively) and the AGR2 mutein of interest (which may again be any of the novel AGR2 proteins or protein fragments described and claimed herein) may be added separately to the above cells in culture. In a preferred embodiment, the label used to monitor cell proliferation is a nucleoside analogue, for example, Bromodeoxyuridine (BrdU), which may be detected via anti-BrdU mouse monoclonal antibodies and subsequent immunofluorescence, immunohistochemical, ELISA or colorimetric methods. Alternatively, 3[H]thymidine incorporation into the cellular DNA and subsequent liquid scintillation chromatography may be used.

A further suitable in vitro assay to determine altered biological activity of a AGR2 mutein according to the present invention compared to wild type mouse Agr2 protein or human AGR2 protein is measuring goblet cell mucus secretion in culture. An assay preferred in this regard is described in Example 21. In such a preferred assay, mammalian goblet cells, and preferably mammalian colon cancer cell lines LS174T or HT29 are transfected with an AGR2 wild type expression vector (e.g., a vector expressing mouse Agr2 protein or human AGR2 protein according to SEQ ID NO:3 and SEQ ID NO:4, respectively), or with an expression vector expressing the AGR2 mutein of interest (i.e., expressing any of the novel AGR2 proteins or protein fragments described and claimed herein). Alternatively, AGR2 wild type protein (e.g., mouse Agr2 protein or human AGR2 protein according to SEQ ID NO:3 and SEQ ID NO:4, respectively) and the AGR2 mutein of interest (which may again be any of the novel AGR2 proteins or protein fragments described and claimed herein) may be added separately to the above cells in culture. Subsequently, the cells are analyzed for changes in the expression of major mucin subtypes secreted by intestinal goblet cells, preferably for the expression of mucin2 (muc2). This can be done, for example, via RT-PCR reverse transcription polymerase chain reaction) using muc2-specific primers and mRNA from transfected and non-transfected or mock-transfected control cells, and subsequent quantitative PCR analysis. Alternatively, or in addition, the cells may be analyzed for changes in the expression of trefoil proteins, again, for example, via RT-PCR using trefoil-specific primers and mRNA from transfected and non-transfected or mock-transfected control cells and subsequent quantitative PCR analysis.

Yet a further suitable in vitro assay to determine altered biological activity of an AGR2 mutein according to the present invention compared to wild type mouse Agr2 protein or human AGR2 protein is measuring Xenopus laevis cement gland differentiation, e.g., as described by Aberger et al. (Aberger et al., 1998). An assay preferred in this regard is described in Example 19. In such a preferred assay, the effect of expression or over-expression of wild type AGR2 protein or AGR2 mutein upon the induction of ectopic cement gland differentiation and expression of anterior neural marker genes in Xenopus embryos is analyzed. In particular, vectors capable of expressing mRNA encoding wild type AGR2 protein (e.g., mouse Agr2 protein or human AGR2 protein according to SEQ ID NO:3 and SEQ ID NO:4, respectively), or mRNA encoding the AGR2 mutein of interest (i.e., encoding any of the novel AGR2 proteins or protein fragments described and claimed herein) are subjected to in vitro transcription, optionally followed by analyzing the quality of the RNA obtained via an in vitro translation system, e.g., reticulocyte lysate, and the capped mRNA thus obtained injected into early cleavage stage embryos of Xenopus laevis. Biological activity is subsequently analyzed by monitoring differentiation of mucin secreting cement glands. For example, biological activity is analyzed by monitoring cement gland enlargement or the presence of additional ectopic cement glands, as described in Aberger et al.

A non-human vertebrate animal according to the present invention is furthermore one wherein the mutated allele contains a mutation which corresponds to a mutation of the human AGR2 protein according to SEQ ID NO:4 which is indicative of an increased risk of a human subject of developing a medical condition associated with an alteration in goblet cell function, or indicative of an association of a medical condition in a human subject which is associated with an alteration in goblet cell function with altered AGR2 expression or function. The term “corresponds to” again refers to the fact that the allele reflects the mutation in the way explained in more detail above. Mutations of the kind contemplated in this regard, and suitable methods of identifying them, are described in more detail below.

In view of the fact that the present invention demonstrates for the first time that AGR2 is required for normal goblet cell function, and that mutating this gene and its gene product may result in goblet cell dysfunction and corresponding physiological and medical disorders of the affected animal, it will be apparent to the skilled artisan that other genes and their products which in turn affect AGR2 gene expression or the function of the AGR2 protein will likewise affect goblet cell-related phenotypes and physiological and medical conditions. Accordingly, the present invention provides in a further aspect a non-human vertebrate animal comprising in the genome of at least some or all of its cells an allele of a gene coding for a protein which affects expression or function of the AGR2 protein of the animal, said allele comprising a mutation which, if present in the genome of all or essentially all cells of said animal in a homozygous manner, results in a phenotype associated with an alteration in goblet cell function compared to the corresponding wild-type animal.

The gene referred to above in connection with the animal according to the invention is preferably an endogenous gene with respect to said animal. In preferred embodiments, the gene will encode a protein which is an orthologue of the AGR2 proteins defined by SEQ ID NO:3 and SEQ ID NO:4 with respect to said animal. The gene may, however, also be a heterologous gene with respect to said animal. For example, a mouse according to the present invention may be one wherein the endogenous mouse Agr2 gene has been replaced by a mutated human AGR2 gene, e.g., by an AGR2 gene encoding a protein according to SEQ ID NO:30. Likewise, a rat according to the present invention may be one wherein the endogenous rat AGR2 gene has been replaced by a mutated mouse Agr2 gene, e.g., by an Agr2 gene encoding a protein according to SEQ ID NO:2.

As will be apparent from the previous explanations, the non-human vertebrate animals according to the invention may also be transgenic animals, i.e., the mutated allele of the gene may represent DNA that is heterologous with respect to the genomic DNA of said animal, or it may be mutated by virtue of the insertion of DNA that is heterologous with respect to the genomic DNA of said animal. Heterologous DNA may be inserted, for example, by the method of targeting vector-mediated homologous recombination at the Agr-2 genomic DNA locus in mouse embryonic stem cells, resulting in a replacement of the endogenous Agr-2 allele by heterologous DNA, as will be appreciated by those skilled in the art. Transgenic animals may then be generated by subsequent breeding.

The endogenous promoter of the AGR2 gene or the gene affecting its expression or function may be replaced by a heterologous promoter, e.g., a promoter imposing a different tissue specificity of expression upon the gene, or a promoter that is inducible by chemical or physical means.

The non-human vertebrate animal according to the invention may also be a “knock-out” animal with respect to the AGR2 gene or the gene affecting expression or function of the AGR2 protein. In these animals, the above-mentioned mutation results in the reduction or complete abolishment of expression of said gene.

The mutated allele may be present in the germ cells or the somatic cells of the non-human vertebrate animal, or both. In a preferred embodiment, the genome of said cells is homozygous with respect to said allele.

The present invention further provides for inbred successive lines of animals carrying the mutant AGR2 nucleic acid of the present invention that offer the advantage of providing a virtually homogenous genetic background. A genetically homogenous line of animals provides a functionally reproducible model system for disorders or symptoms associated with alterations in goblet cell function and mucosal epithelium.

In a particularly preferred embodiment the non-human vertebrate animal according to the invention expresses in at least some of its cells, preferably the goblet cells, a polypeptide as shown in SEQ ID NO:2 or SEQ ID NO:30.

The animals of the invention can be produced by using any technique known to the person skilled in the art; including but not limited to micro-injection, electroporation, cell gun, cell fusion, micro-injection into embryos of teratocarcinoma stem cells or functionally equivalent embryonic stem cells. The animals of the present invention may be produced by the application of procedures, which result in an animal with a genome that incorporates/integrates exogenous genetic material in such a manner as to modify or disrupt the function of the normal AGR2 gene or protein. A preferred procedure for generating an animal of this invention is one according to Example 1.

Alternatively, the procedure may involve obtaining genetic material, or a portion thereof, which encodes a wild type AGR2 protein, as described in Example 5. The isolated native sequence is then genetically manipulated by the insertion of any of the mutations described and claimed in accordance with the present invention, e.g., a mutation appropriate to replace, e.g., the residue at position 137 of the amino acid sequence shown in SEQ ID NO:3 or SEQ ID NO:4. The manipulated construct may then be inserted into embryonic stem cells, e.g., by electroporation. The cells subjected to the procedure are screened to find positive cells, i.e., cells, which have integrated into their genome the desired construct encoding an altered AGR2. The positive cells may be isolated, cloned (or expanded) and injected into blastocysts obtained from a host animal of the same species or a different species. For example, positive cells are injected into blastocysts from mice, the blastocysts are then transferred into a female host animal and allowed to grow to term, following which the offspring of the female are tested to determine which animals are transgenic, i.e., which animals have an inserted exogenous mutated DNA sequence. One suitable method involves the introduction of the recombinant gene at the fertilized oocyte stage ensuring that the gene sequence will be present in all of the germ cells and somatic cells of the “founder” animal. The term “founder animal” as used herein means the animal into which the recombinant gene was introduced at the one cell embryo stage.

The animals of the invention can also be used as a source of primary cells from a variety of tissues, for cell culture experiment, including, but not limited to, the production of immortalized cell lines by any methods known in the art, such as retroviral transformation. Such primary cells or immortalized cell lines derived from any one of the non-human vertebrate animals described and claimed herein are likewise within the scope of the present invention. Such immortalized cells from these animals may advantageously exhibit desirable properties of both normal and transformed cultured cells, i.e., they will be normal or nearly normal morphologically and physiologically, but can be cultured for long, and perhaps indefinite periods of time. The primary cells or cell lines derived thereof may furthermore be used for the construction of an animal model according to the present invention.

In other embodiments cell lines according to the present invention may be prepared by the insertion of a nucleic acid construct comprising the nucleic acid sequence of the invention or a fragment thereof comprising the codon imparting the above-described phenotype to the animal model of the invention. Suitable cells for the insertion include primary cells harvested from an animal as well as cells, which are members of an immortalized cell line. Recombinant nucleic acid constructs of the invention, described below, may be introduced into the cells by any method known in the art, including but not limited to, transfection, retroviral infection, micro-injection, electroporation, transduction or DEAE-dextran. Cells, which express the recombinant construct, may be identified by, for example, using a second recombinant nucleic acid construct comprising a reporter gene, which is used to produce selective expression. Cells that express the nucleic acid sequence of the invention or a fragment thereof may be identified indirectly by the detection of reporter gene expression.

It will be appreciated that the non-human vertebrate animals of the invention are useful in various respects in connection with goblet cell function or dysfunction and goblet cell-related phenotypes and medical conditions.

Accordingly, one aspect of the present invention is the use of the non-human vertebrate animal for the identification of a protein or nucleic acid diagnostic marker for a goblet cell-related disorder. Also within the scope of the present invention is the use of the animal as a model for studying the molecular mechanisms of, or physiological processes associated with, a goblet cell-related disorder.

Furthermore, the non-human vertebrate animal of the present invention may be used for the identification and testing of agents useful in the prevention, amelioration, or treatment of a goblet cell-related disorder. Such goblet cell-related disorders are in particular asthma, chronic obstructive pulmonary disease (COPD), cystic fibrosis, dry eye syndrome, gastric disease, peptic ulcer, inflammatory bowel disease (in particular Crohn's disease or ulcerative colitis), and intestinal cancer.

Further uses of the non-human vertebrate animals described herein which form additional aspects of the present invention are those relating to studying the molecular mechanisms of, or physiological processes associated with, conditions associated with, or affected by, reduced activity or undesirable, e.g., increased, activity of endogenous AGR2. Likewise, conditions associated with reduced expression, reduced production or undesirable, e.g., increased production of endogenous AGR2 may be analyzed.

It will also be appreciated that the non-human vertebrate animals described herein will be highly useful as a model system for the screening, identification and testing of agents useful in the prevention, amelioration, or treatment of the above-mentioned conditions. Such agents may be, for example, small molecule drugs, peptides or polypeptide, or nucleic acids. For the purposes of the present invention, small molecule drugs preferably have a molecular weight of no more than 2,000 Dalton, more preferably no more than 1500 Dalton, even more preferably no more than 1000 Dalton, and most preferably no more than 500, 400, 300 or even 200 Dalton. Such agents may alter the biological activity of the wild type AGR2 or the AGR2 mutein, i.e., these agents may act on both types of proteins as agonist or antagonist.

It will furthermore be apparent from the above that the non-human vertebrate animals described herein will be highly useful for identifying protein or nucleic acid diagnostic markers, such as diagnostic markers relating to genes or gene products that play a role in the early phase, the intermediate phase, and/or the late phase of medical conditions associated with an alteration in goblet cell function, e.g., for diseases associated with wild type AGR2 or AGR2 mutein deficiency or over-expression. It will be appreciated that such diagnostic markers may relate to the AGR2 gene or its protein product. However, it will be appreciated that the non-human vertebrate animal according to the present invention can also be used to identify markers relating to other genes or gene products that affect AGR2 gene or protein expression or function, or the expression or function of which is affected by the AGR2 protein. Moreover, since the non-human vertebrate animal of the invention represents a highly useful model system for studying the pathogenesis of medical conditions associated with an alteration in goblet cell function, it will be appreciated that it may also be used to identify disease-relevant markers relating to genes or gene products that do not directly affect AGR2 gene or protein expression or function, or the expression or function of which is not directly affected by the AGR2 protein. It will be appreciated that the above-mentioned uses represent further aspects of the present invention.

Finally, it will be appreciated from the above that the non-human vertebrate animals described herein will be highly useful for identifying receptors of the AGR2 protein, or upstream or downstream genes or proteins regulated by the AGR2 protein or gene activity, and deregulated in disorders associated with AGR2 deficiency or over-expression.

### Nucleic Acids

The present invention furthermore provides nucleic acid sequences encoding the AGR2 muteins as described in more detail below, for example murine and human AGR2 mutated in accordance with the present invention. In a preferred embodiment, this invention provides a mutated nucleic acid sequence for murine AGR2 (SEQ ID NO:1). Furthermore, this invention provides a mutated nucleic acid sequence of human AGR2 (SEQ D NO:29). Mutated human AGR2 genes can be made, for example, by altering codon 137 of the wild type human AGR2 gene (SEQ ID NO:5), such that codon 137 no longer encodes valin. The construction of a gene with a 137^{th} codon that does not encode valin is well known. Valin is encoded by GTT, GTC, GTA and GTG. A codon that does not encode valin may be, for example, a codon that encodes Phe (TTT, TTC); Leu (TTA, TTG, CTT, CTC, CTA, CTG); Ile (ATT, ATC, ATA); Met (ATG); Asp (GAC, GAT); Ser (TCT, TCC, TCA, TCG), Pro (CCT, CCC, CCA, CCG); Thr (ACT, ACC, ACA, ACG), Ala (GCT, GCC, GCA, GCG); Tyr (TAT, TAC); His (CAT, CAC), Gln (CAA, CAG); Asn (AAT, AAC); Lys (AAA, AAG); Glu (GAA, GAG); Cys (TGT, TGC); Trp (TGG); Arg (CGT, CGC, CGA, CGG, AGA, AGG); Ser (AGT, AGC); Gly (GGT, GGC, GGA, GGG) or one of the stop codons (TAA, TAG, TGA). Methods for the introduction of site-specific nucleic acid mutations are well known.

The nucleic acid sequences encoding mutant AGR2 of the invention may exist alone or in combination with other nucleic acids as, for example, vector molecules, such as plasmids, including expression or cloning vectors.

The term “nucleic acid sequence” as used herein refers to any contiguous sequence series of nucleotide bases, i.e., a polynucleotide, and is preferably a ribonucleic acid (RNA) or deoxy-ribonucleic acid (DNA). Preferably the nucleic acid sequence is cDNA. It may, however, also be, for example, a peptide nucleic acid (PNA).

An “isolated” nucleic acid molecule, as referred to herein, is one, which is separated from other nucleic acid molecules ordinarily present in the natural source of the nucleic acid. Preferably, an “isolated” nucleic acid is free of sequences, which naturally flank the nucleic acid (i.e., sequences located at the 5′- and 3′-termini of the nucleic acid) in the genomic DNA of the organism that is the natural (wild type) source of the DNA.

AGR2 gene molecules can be isolated using standard hybridization and cloning techniques, as described, for instance, in Sambrook et al. (eds.), MOLECULAR CLONING: A LABORATORY MANUAL (2^{nd} Ed.), Cold Spring. Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993.

A nucleic acid of the invention can be amplified using cDNA, mRNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to AGR2 nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Generally, the term “oligonucleotide” is used to refer to a series of contiguous nucleotides (a polynucleotide) of about 100 nucleotides (nt) or less, e.g., portions of a nucleic acid sequence of about 100 nt, 50 nt, or 20 nt in length, preferably nucleotide sequences of about 15 nt to 30 nt in length.

As used herein, the term “complementary” refers to Watson-Crick or Hoogsteen base pairing between nucleotide units of a nucleic acid molecule, and the term “binding” means the physical or chemical interaction between two polypeptides or compounds or associated polypeptides or compounds or combinations thereof.

A “homologous nucleic acid sequence” or “homologous amino acid sequence,” or variations thereof, refers to sequences characterized by a homology at the nucleotide level or amino acid level, respectively. Homologous nucleotide sequences can include those sequences coding for isoforms of AGR2 polypeptides. Isoforms can be expressed in different tissues of the same organism as a result of, for example, alternative splicing of RNA. Alternatively, isoforms can be encoded by different genes.

As used herein, the phrase “stringent hybridization conditions” refers to conditions under which a probe, primer or oligonucleotide or any other nucleic acid sequence referred to herein will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tn, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3, and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide. Stringent conditions are known to those skilled in the art and can be found in Ausubel et al. (eds.), CURRENT PROTOCOLS IN MOLECULAR BIOLOGY, John Wiley & Sons, N.Y. (1989), 6.3.1-6.3.6.

Preferred stringent hybridization conditions in accordance with the nucleic acids of the present invention, for example the antisense nucleic acids described further below, are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

As used herein, for example, in connection with the antisense nucleic acids of the present invention described further below, the phrase “hybridization under physiological conditions” refers to hybridization of a probe, primer or oligonucleotide, or any other nucleic acid sequence to its target sequence under conditions as they are found inside eukaryotic cells either within a multicellular organism or under conditions of cell or tissue culture. Such conditions are preferably characterized by a temperature of about or exactly 37° C., absence of formamide, and an ionic strength corresponding to physiological buffer.

### Antisense Nucleic Acids

A preferred nucleic acid according to the present invention is an antisense nucleic acid comprising a nucleotide sequence which is complementary to a part of an mRNA encoding a mutein according to the present invention, said part encoding an amino acid sequence comprising the amino acid or amino acid sequence which corresponds to the mutation described in more detail in connection with said muteins.

A further preferred antisense nucleic acid is one comprising a nucleotide sequence which is complementary to a part of an mRNA encoding the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein as defined above, said part being a non-coding part and comprising a sequence corresponding to a mutation in the gene coding for said protein or orthologue which affects expression of said protein or orthologue.

Yet a further preferred antisense nucleic acid is one comprising a nucleotide sequence which is complementary to a part of an mRNA encoding a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

In a preferred embodiment, the antisense nucleic acid is capable of hybridizing to the mRNA via the complementary nucleotide sequence under physiological conditions, in particular the preferred physiological conditions defined above. In this case, the antisense RNA is inter alia suitable to be used in connection with the methods and uses of the present invention that relate to the prevention, treatment, or amelioration of a medical condition associated with an alteration in goblet cell function. In another preferred embodiment, the antisense RNA according to the present invention is capable of hybridizing to said mRNA under high stringency conditions, in particular the preferred high stringency conditions defined above.

The antisense nucleic acid may be a ribozyme comprising a catalytic region; suitably, the catalytic region enables the antisense RNA to specifically cleave the mRNA to which the antisense RNA hybridizes.

It may be advantageous that the antisense nucleic acid of the invention hybridizes more effectively to its target mRNA than to an mRNA encoding the same protein which, however, corresponds to the wild-type mouse Agr2 or human AGR2 protein according to SEQ ID NO:3 and SEQ ID NO:4 in respect of the mutated amino acid sequence. Also preferred are antisense nucleic acids which hybridize more effectively to their target mRNA than to the mRNA encoded by the wild-type genes encoding the mouse Agr2 protein or the human AGR2 protein according to SEQ ID NO:3 and SEQ ID NO:4, respectively, or the wild-type gene encoding the corresponding orthologue. Preferred are in addition antisense nucleic acids which hybridize more effectively to their target mRNA than to the mRNA encoded by the wild-type gene of the corresponding protein which affects expression or function of the mouse Agr2 or the human AGR2 protein according to SEQ ID NO:3 and SEQ ID NO:4, respectively.

Prokaryotic and eukaryotic host cells transformed with the above antisense nucleic acids are likewise within the scope of the present invention.

### Aptamers

Aptamers are macromolecules composed of nucleic acid, such as RNA or DNA, that tightly bind to protein. The present invention provides aptamers specifically binding to the proteins described herein. Preferably, the specificity of the aptamers is sufficient so that they do not, or substantially do not, bind to any other protein in the cell. Preferred aptamers bind to the AGR2 muteins of the present invention, or a portion thereof comprising a mutation as described herein, e.g., a substitution of amino acid 137. Another preferred aptamer binds to the wild type AGR2 protein or a portion thereof. The aptamers of the present invention preferably bind their ligands with high specificity and affinity in the nanomolar range, e.g., in the low nanomolar range with K(D) values ranging between 12 nM and 130 nM.

### Interfering RNA

In one aspect of the invention, AGR2 gene expression can be attenuated by RNA interference. One approach well-known in the art is short interfering RNA (siRNA) mediated gene silencing where expression products of a AGR2 gene are targeted by specific double stranded AGR2 derived siRNA nucleotide sequences that are complementary to at least a 19-25 nt long segment of the AGR2 gene transcript, including the 5′ untranslated (UT) region, the open reading frame (ORF), or the 3′ UT region. See, for example, PCT applications WO00/44895, WO99/32619, WO01/5164, WO01/92513, WO01/29058, WO01/89304, WO02/16620, and WO02/29858, each incorporated by reference herein in their entirety. Targeted genes can be an AGR2 gene, or an upstream or downstream modulator of AGR2 gene expression or protein activity. For example, expression of a phosphatase or kinase of AGR2 may be targeted by an siRNA.

According to the methods of the present invention, AGR2 gene expression is silenced using short interfering RNA. An AGR2 polynucleotide according to the invention includes an siRNA polynucleotide. Such an AGR2 siRNA can be obtained using an AGR2 polynucleotide sequence, for example, by processing the AGR2 ribopolynucleotide sequence in a cell-free system, such as but not limited to a Drosophila extract, or by transcription of recombinant double stranded AGR2 RNA or by chemical synthesis of nucleotide sequences homologous to a AGR2 sequence. See, e.g., Tuschl, Zamore, Lehmann, Bartel and Sharp (1999), Genes & Dev. 13: 3191-3197, incorporated herein by reference in its entirety (Tuschl et al., 1999). When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxynucleotides in the 3′ overhangs is as efficient as using ribonucleotides, but deoxyribonucleotides are often cheaper to synthesize and are most likely more nuclease resistant.

A recombinant expression vector of the invention comprises a AGR2 DNA molecule cloned into an expression vector comprising operatively-linked regulatory sequences flanking the AGR2 sequence in a manner that allows for expression (by transcription of the DNA molecule) of both strands. An RNA molecule that is antisense to AGR2 mRNA is transcribed by a first promoter (e.g., a promoter sequence 3′ of the cloned DNA) and an RNA molecule that is the sense strand for the AGR2 mRNA is transcribed by a second promoter (e.g., a promoter sequence 5′ of the cloned DNA). The sense and antisense strands may hybridize in vivo to generate siRNA constructs for silencing of the AGR2 gene. Alternatively, two constructs can be utilized to create the sense and anti-sense strands of an siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is an siRNA. The regulatory sequences flanking the AGR2 sequence may be identical or may be different, such that their expression may be modulated independently, or in a temporal or spatial manner.

In a specific embodiment, siRNAs are transcribed intracellularly by cloning the AGR2 gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

siRNA vectors appear to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (sIRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

An AGR2 mRNA region to be targeted by siRNA is generally selected from a desired AGR2 sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See Elbashir et al. 2001 EMBO J. 20(23):6877-88 (Elbashir et al., 2001b). Hence, consideration should be taken to accommodate SNPs, polymorphisms, allelic variants or species-specific variations when targeting a desired gene.

A complete AGR2 siRNA experiment should include the proper negative control. Negative control siRNA should have the same nucleotide composition as the AGR2 siRNA but lack significant sequence homology to the genome. Typically, one would scramble the nucleotide sequence of the AGR2 siRNA and do a homology search to make sure it lacks homology to any other gene.

Two independent AGR2 siRNA duplexes can be used to knock-down a target AGR2 gene. This helps to control for specificity of the silencing effect. In addition, expression of two independent genes can be simultaneously knocked down by using equal concentrations of different AGR2 siRNA duplexes. Availability of siRNA-associating proteins is believed to be more limiting than target mRNA accessibility.

A targeted AGR2 region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the AGR2 sense siRNA corresponds to (N19)TT or N21, respectively. In the latter case, conversion of the 3′ end of the sense siRNA to TT can be performed if such a sequence does not naturally occur in the AGR2 polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs (see, Elbashir, Lendeckel and Tuschl (2001), Genes & Dev. 15: 188-200, incorporated by reference herein in its entirely) (Elbashir et al., 2001a). The modification of the overhang of the sense sequence of the siRNA duplex is not expected to affect targeted mRNA recognition, as the antisense siRNA strand guides target recognition.

Alternatively, if the AGR2 target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See Harborth et al. (2001) J. Cell Science 114: 4557-4565, incorporated herein by reference in its entirety (Harborth et al., 2001).

Transfection of AGR2 siRNA duplexes can be achieved using standard nucleic acid transfection methods, for example, OLIGOFECTAMINE Reagent (commercially available from Invitrogen). An assay for AGR2 gene silencing is generally performed approximately 2 days after transfection. No AGR2 gene silencing has been observed in the absence of transfection reagent, allowing for a comparative analysis of the wild type and silenced AGR2 phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful AGR2 silencing. The efficiency of transfection needs to be carefully examined for each new cell line to be used. Preferred cells are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense AGR2 siRNA will have no effect on AGR2 silencing, and 0.84 μg antisense siRNA has a weak silencing effect when compared to 0.84 μg of duplex siRNAs. Control experiments again allow for a comparative analysis of the wild type and silenced AGR2 phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted AGR2 polynucleotide in a cell, a knock-down phenotype may become apparent after 1 to 3 days, or even later. In cases where no AGR2 knock-down phenotype is observed, depletion of the AGR2 polynucleotide may be observed by immunofluorescence or Western blotting. If the AGR2 polynucleotide is still abundant after 3 days, cells need to be split and transferred to a fresh 24-well plate for re-transfection. If no knock-down of the targeted protein (AGR2 or a AGR2 upstream or downstream gene) is observed, it may be desirable to analyze whether the target mRNA was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable AGR2 protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering an AGR2 siRNA construct as therapy to compensate for increased or aberrant AGR2 expression or activity. The AGR2 ribopolynucleotide is obtained and processed into siRNA fragments as described. The AGR2 siRNA is administered to cells or tissues using known nucleic acid transfection techniques, as described above. An AGR2 siRNA specific for an AGR2 gene will decrease or knockdown AGR2 transcription products, which will lead to reduced AGR2 polypeptide production, resulting in reduced AGR2 polypeptide activity in the cells or tissues.

Particularly preferred in connection with the present invention are siRNAs comprising a double stranded nucleotide sequence wherein one strand is complementary to an at least 19, 20, 21, 22, 23, 24, or 25 nucleotide long segment of an mRNA encoding a mutein of the invention as described herein, said segment encoding an amino acid sequence comprising the amino acid or amino acid sequence which corresponds to any of the mutations defined previously in connection with these muteins.

Also preferred are siRNAs wherein said strand is complementary to an at least 19, 20, 21, 22, 23, 24, or 25 nucleotide long segment of an mRNA encoding the mouse Agr2 or the human AGR2 protein according to SEQ ID NO:3 and SEQ ID NO:4, respectively, or an orthologue thereof having or at least 65%, 70%, 75%, 80%, 85%, 90%, 95%, 98%, or 99% amino acid identity compared to the mouse Agr2 or the human AGR2 protein as defined above, said segment being a non-coding segment and comprising a sequence corresponding to a mutation in the gene coding for said protein or orthologue which affects expression of said protein or orthologue.

Furthermore preferred are siRNAs wherein said strand is complementary to an at least 19, 20, 21, 22, 23, 24, or 25 nucleotide long segment of an mRNA encoding a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

The above-mentioned segment may include sequences from the 5′ untranslated (UT) region. Alternatively, or in addition, it may include sequences corresponding to the open reading frame (ORF). Again alternatively or in addition, it may include sequences from the 3′ untranslated (UT) region.

Prokaryotic and eukaryotic host cells transformed with the above siRNAs are likewise within the scope of the present invention.

The present invention also encompasses a method of treating a disease or condition associated with the presence of an AGR2 protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the AGR2 gene function is not correlated with a known phenotype, a control sample of cells or tissues from healthy individuals provides a reference standard for determining AGR2 expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The AGR2 ribopolynucleotide is used to produce siRNA constructs, that are specific for the AGR2 gene product. These cells or tissues are treated by administering AGR2 siRNAs to the cells or tissues by methods described for the transfection of nucleic acids into a cell or tissue, and a change in AGR2 polypeptide or polynucleotide expression is observed in the subject sample relative to the control sample, using the assays described. This AGR2 gene knockdown approach provides a rapid method for determination of a AGR2-phenotype in the treated subject sample. The AGR2-phenotype observed in the treated subject sample thus serves as a marker for monitoring the course of a disease state during treatment.

### Proteins and Amino Acids

The present invention also provides, for example, murine and human mutated AGR2 amino acid sequences (muteins). The wild type murine and human amino acid sequences are shown in SEQ ID NO:3 and SEQ ID NO:4 respectively. A mutated version of the mouse amino acid sequence wherein valin at position 137 is mutated to a glutamic acid is exemplified in SEQ ID NO:2. A mutated version of the human amino acid sequence wherein valin at position 137 is mutated to a glutamic acid is exemplified in SEQ ID NO:30.

More generally, the present invention provides a protein having at least 65%, at least 70%, at least 75%, at least 80%, at least 85%, at least 90%, at least 95%, at least 98%, or at least 99% amino acid identity compared to the mouse Agr2 or the human AGR2 protein according to SEQ ID NO:3 and SEQ ID NO:4, respectively. Also encompassed by the present invention are fragments of such proteins comprising at least 6, at least 7, at least 8, at least 9, at least 10, at least 15, at least 20, at least 25, at least 30, at least 35, at least 40, at least 50, at least 60, at least 70, at least 80, at least 90, at least 100, at least 110, at least 120, at least 130, at least 140, at least 150, at least 160, at least 165, at least 170, at least 171, at least 172, at least 173, or at least 174 contiguous amino acids having the above percentages of amino acid identity compared to the corresponding amino acids in SEQ ID NO:3 and SEQ ID NO:4.

In accordance with the invention described herein, the above protein or protein fragment comprises an amino acid or an amino acid sequence which corresponds to a mutation in the mouse Agr2 protein according to SEQ ID NO:3 which, if encoded by the mouse Agr2 gene and present in the genome of all or essentially all cells of a mouse in a homozygous manner, results in a phenotype associated with an alteration in goblet cell function compared to the corresponding wild-type animal.

In an alternative embodiment, the protein or protein fragment comprises an amino acid or an amino acid sequence which corresponds to a mutation in the mouse Agr2 protein or the human AGR2 protein according to SEQ ID NO:3 and SEQ ID NO:4, respectively, which leads to an altered biological activity of the mutated protein when compared to the corresponding wild-type mouse Agr2 protein or human AGR2 protein in an in vitro assay. In vitro assays contemplated in this regard are, for example, those already explained in detail in connection with the non-human vertebrate animal above.

In yet a further alternative embodiment, the protein or protein fragment comprises an amino acid or an amino acid sequence which corresponds to a mutation of the human AGR2 protein according to SEQ ID NO:4 which is indicative of an increased risk of a human subject of developing a medical condition associated with an alteration in goblet cell function, or indicative of an association of a medical condition in a human subject which is associated with an alteration in goblet cell function with altered AGR2 expression or function. The term “corresponds to” as used in the present and the preceding paragraphs refers to the fact that the allele reflects the mutation in the way explained previously in the present specification. Also, a mutation of the human AGR2 protein according to SEQ ID NO:4 referred to in the present paragraph is again of the kind described in more detail elsewhere herein, and identifiable by the methods described and claimed in the present specification.

In a preferred embodiment, the protein of the invention represents an orthologue of the mouse Agr2 or the human AGR2 protein according to SEQ ID NO:3 and SEQ ID NO:4, preferably a vertebrate orthologue, in particular an orthologue wherein said vertebrate is an amphibian vertebrate, in particular Xenopus leavis. Alternatively, it may represent a mammalian orthologue, in particular a rat, rabbit, hamster, dog, cat, sheep, or horse orthologue. It may also be a variant of the mouse Agr2 protein or the human AGR2 protein according to SEQ ID NO:3 and SEQ ID NO:4, respectively, or of said orthologue, allelic or otherwise, wherein certain amino acids or partial amino acid sequences have been replaced, added, or deleted.

Again in a preferred embodiment, the mutation mentioned above results in a deletion or substitution by another amino acid of an amino acid of said mouse Agr2 protein or human AGR2 protein according to SEQ ID NO:3 and SEQ ID NO:4, respectively. Alternatively, the mutation may result in an insertion of additional amino acids not normally present in the amino acid sequence of the mouse Agr2 protein or the human AGR2 protein defined above.

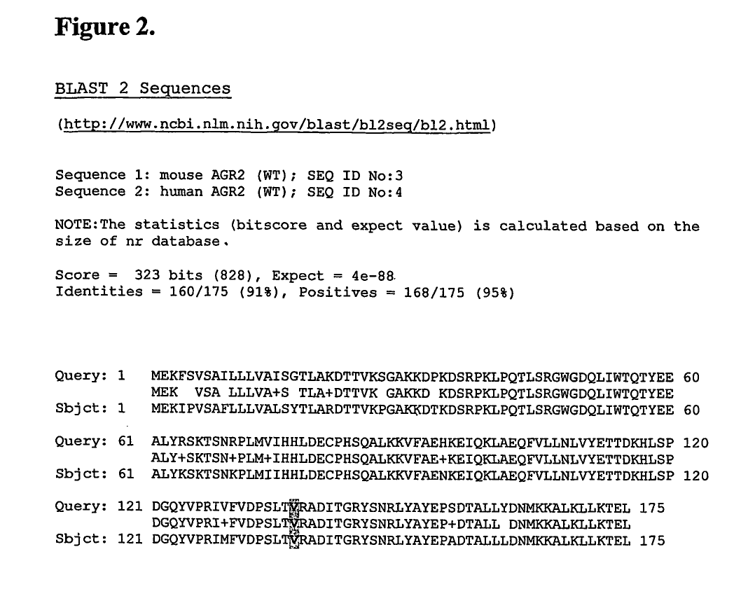

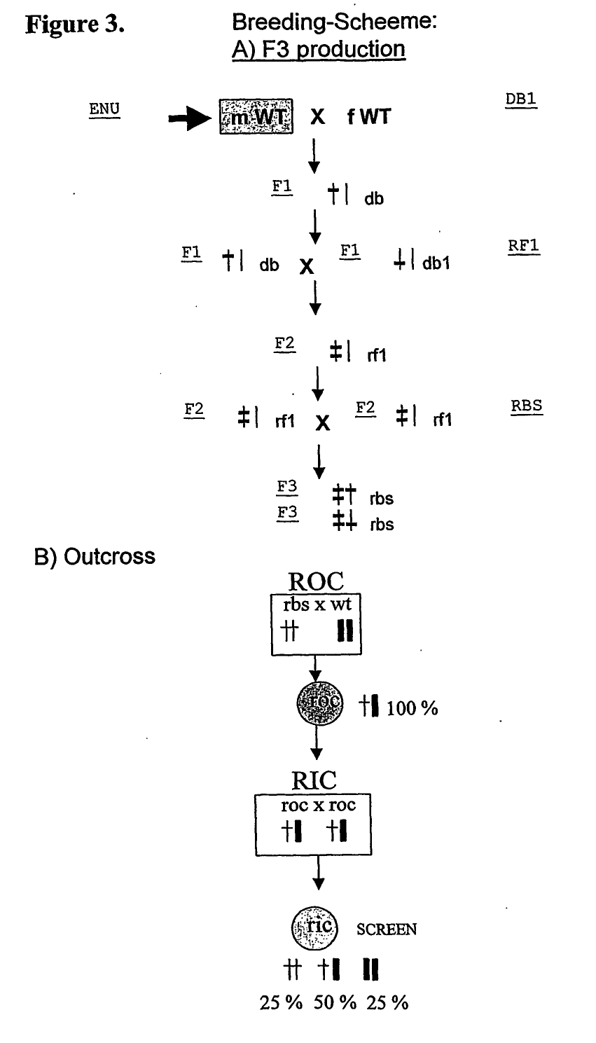

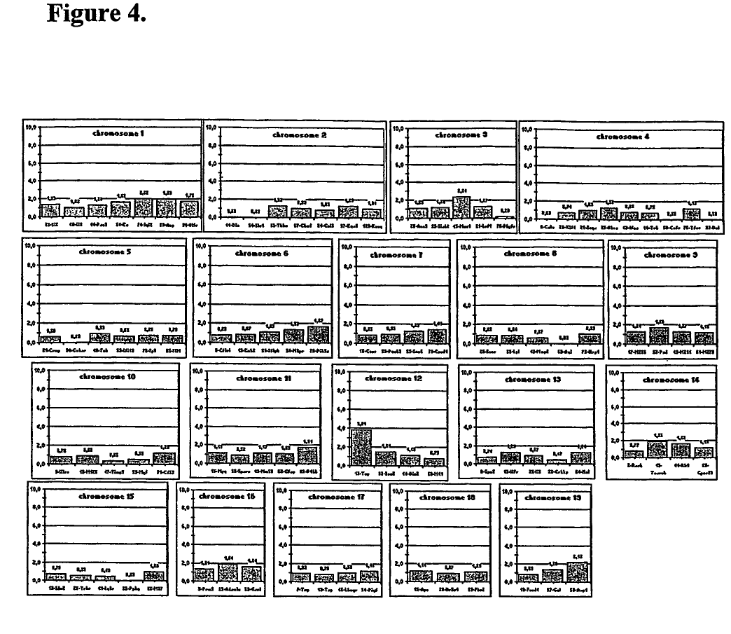

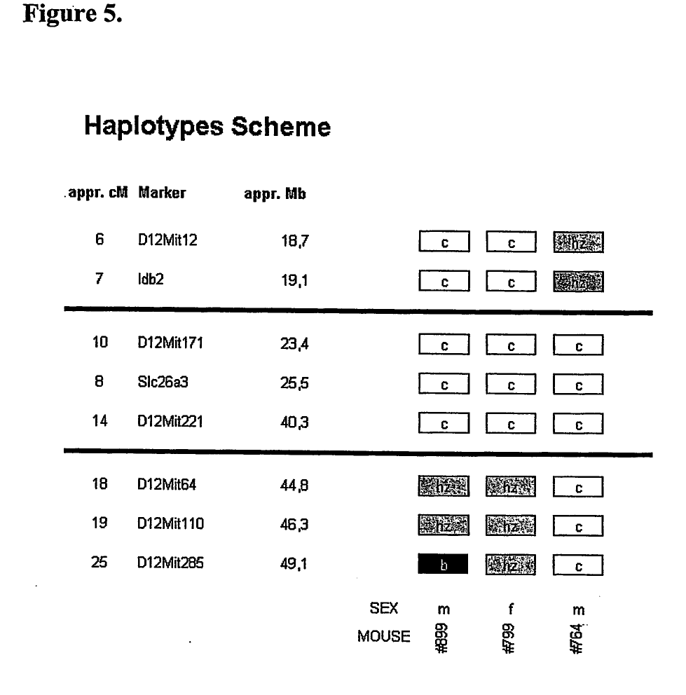

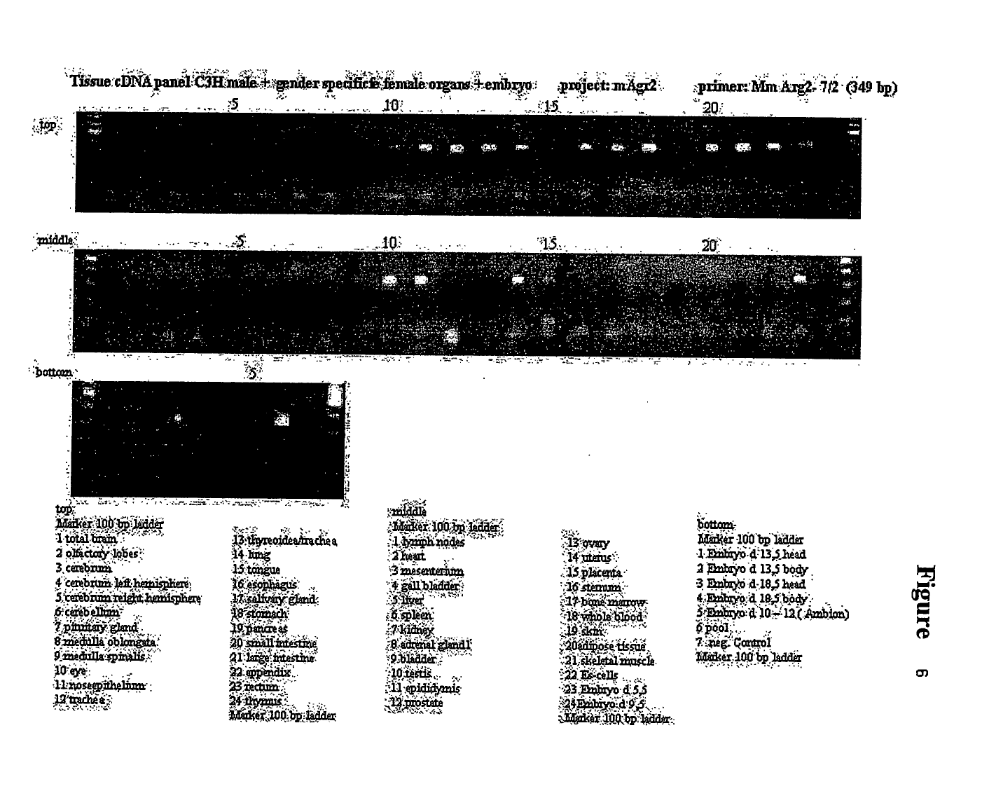

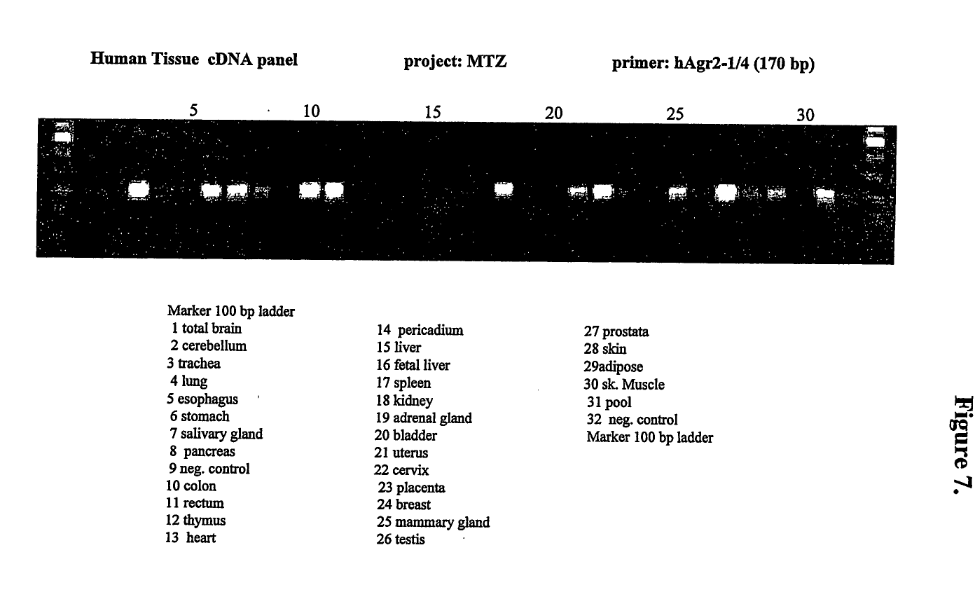

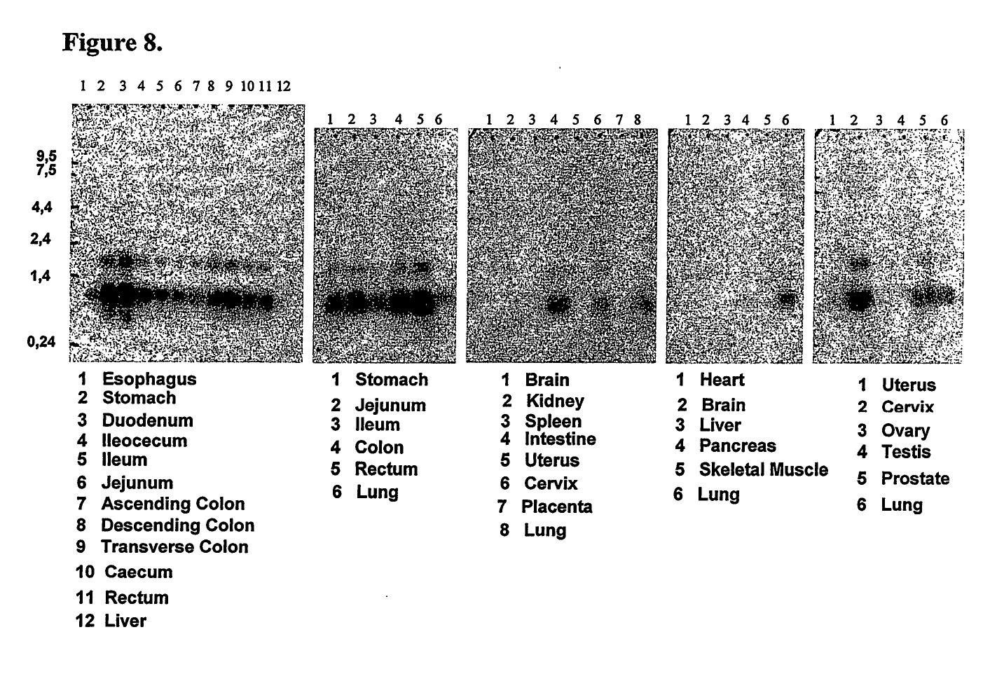

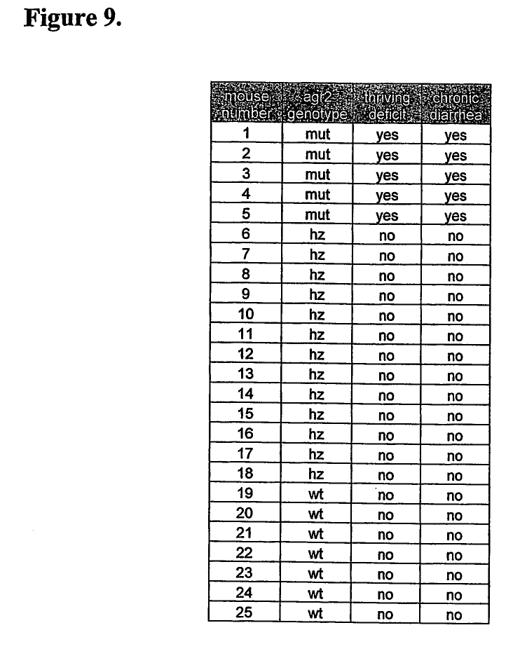

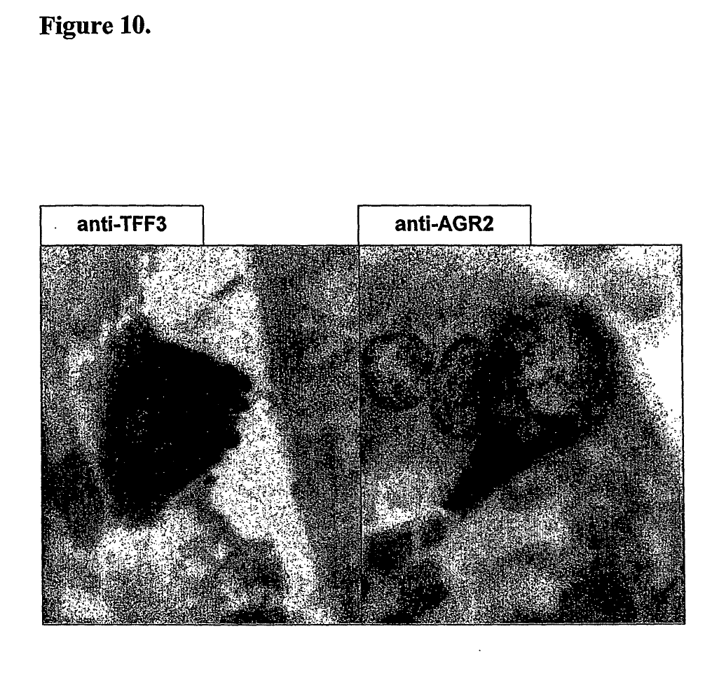

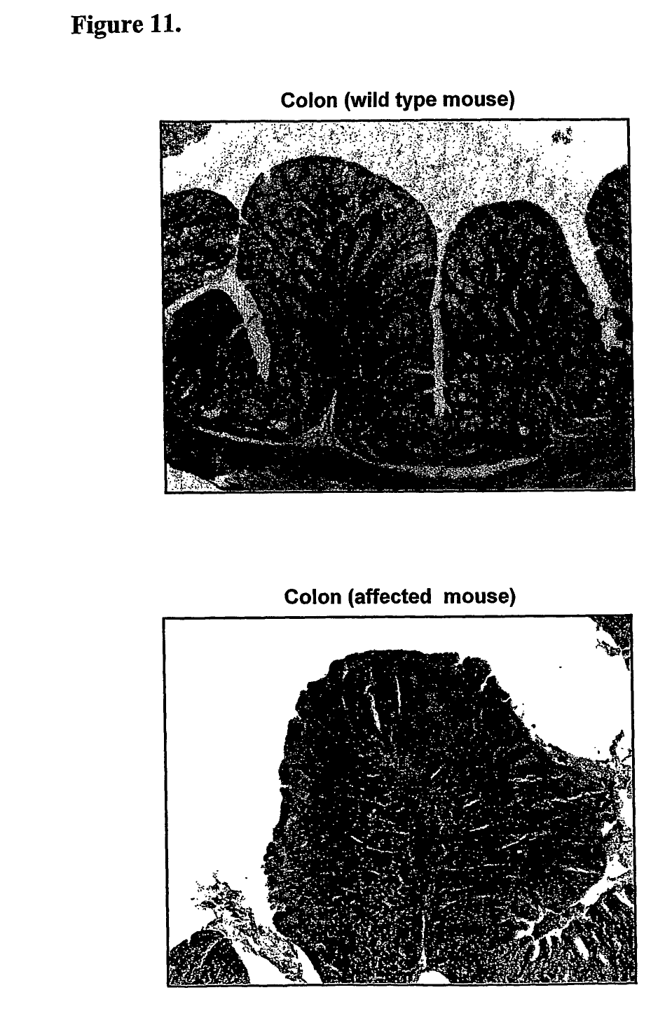

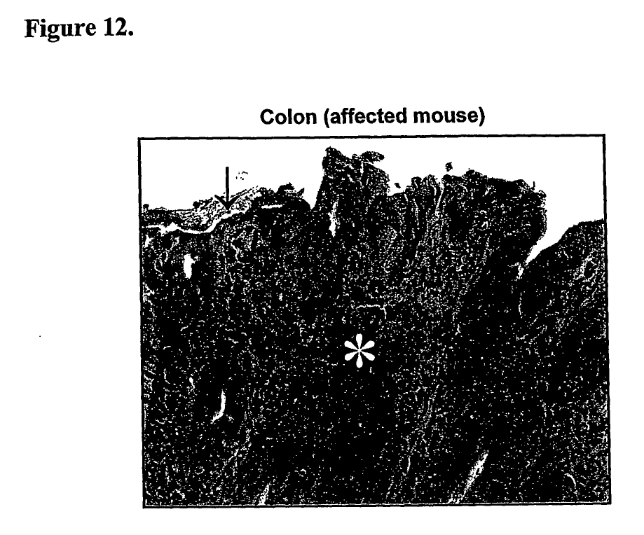

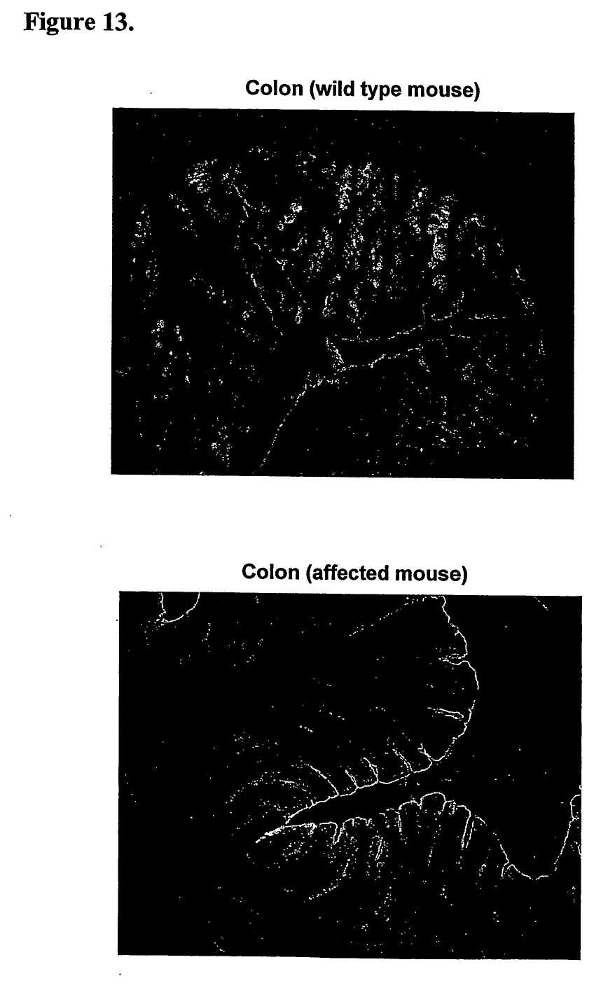

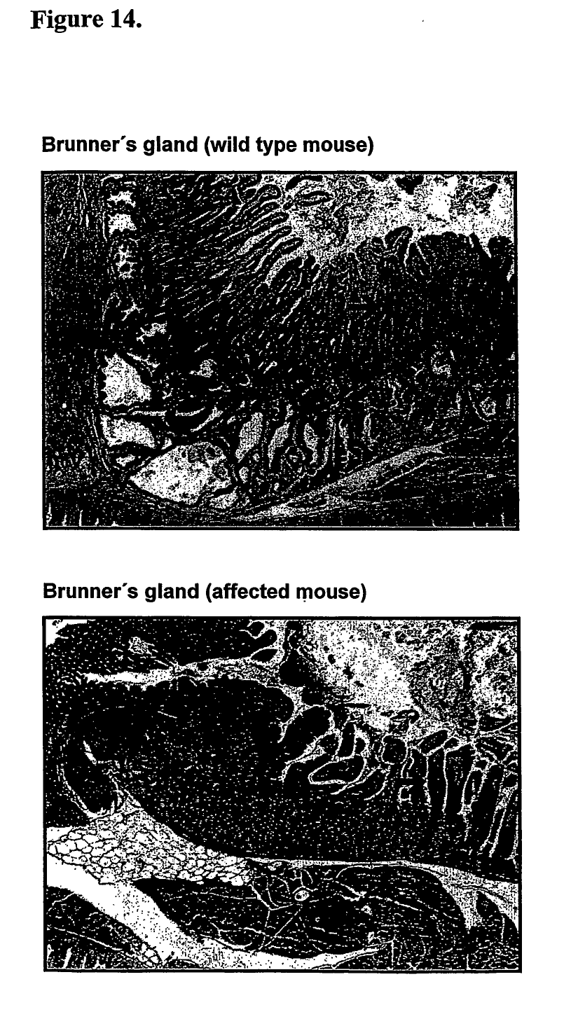

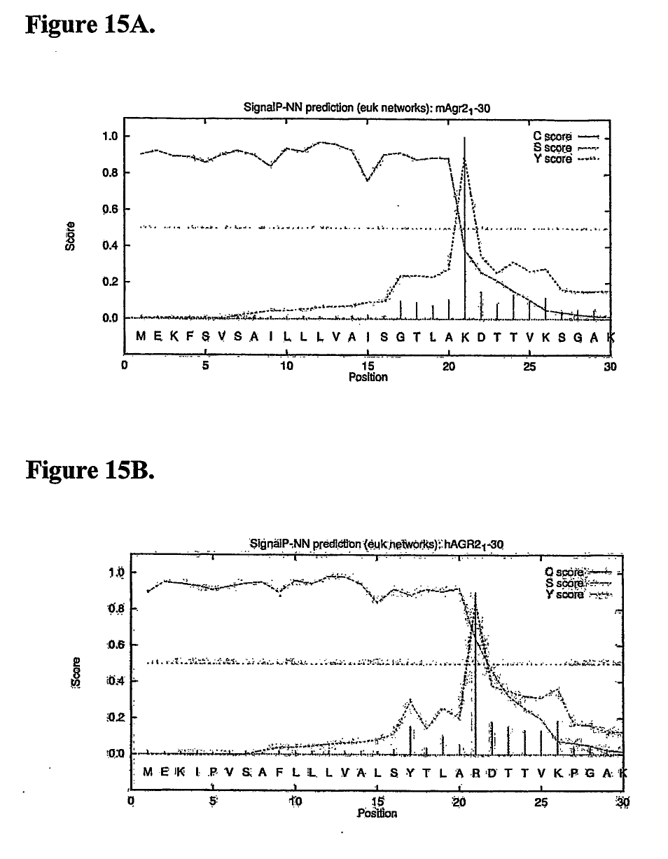

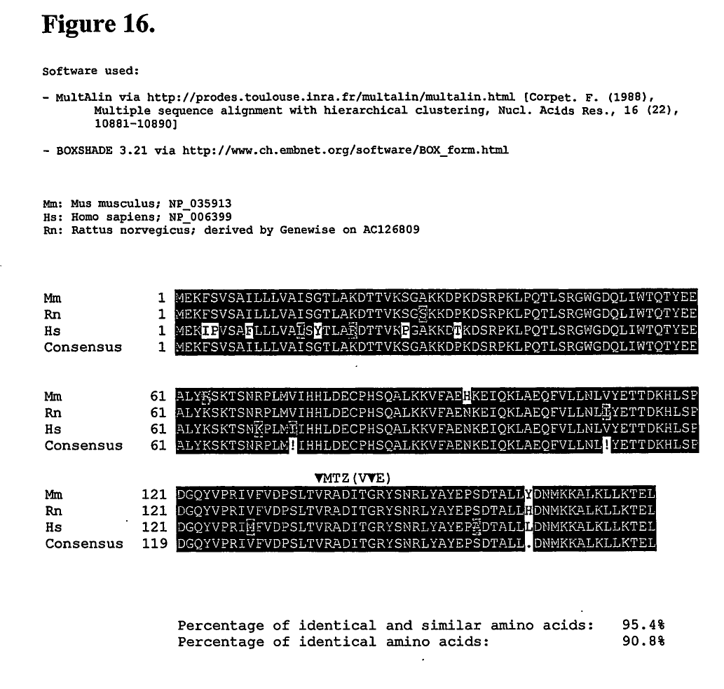

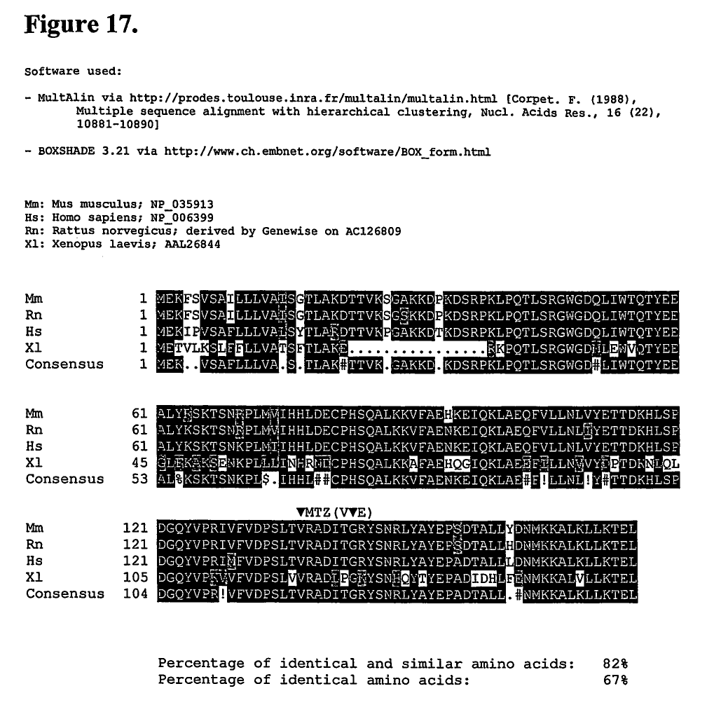

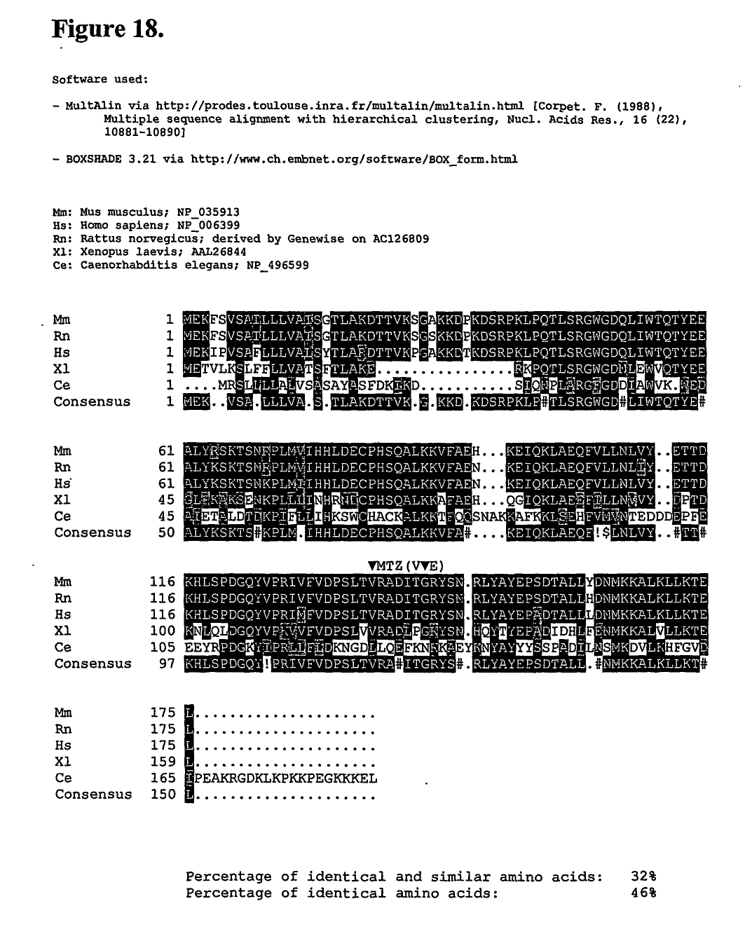

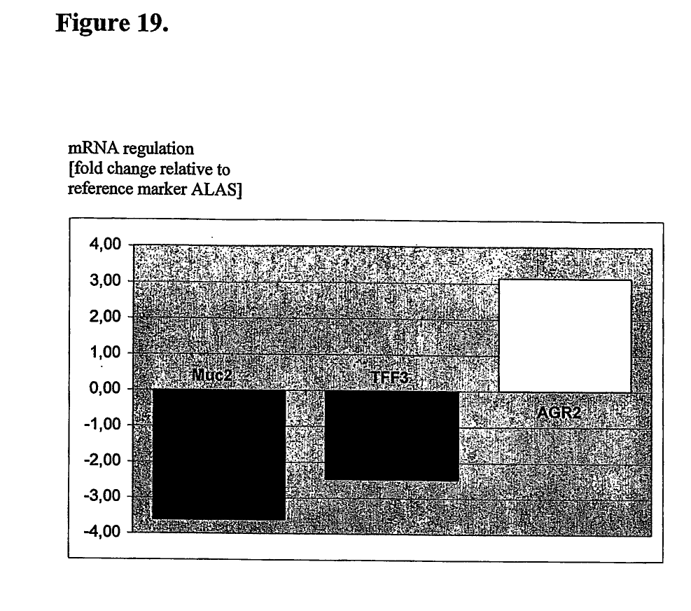

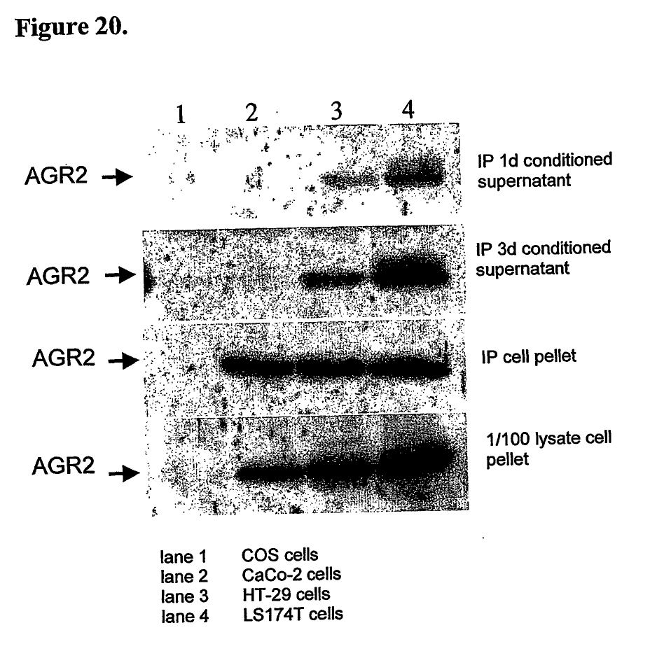

The deletion, substitution, or insertion may furthermore occur in an evolutionary conserved region of said mouse Agr2 protein or said human AGR2 protein. In particular, it may be a substitution of an amino acid which is identical or similar between mouse, rat, and human AGR2, preferably between mouse, rat, human, and Xenopus laevis AGR2, more preferably between mouse, rat, human, Xenopus laevis, and Caenorhabditis elegans AGR2, by another amino acid. Such amino acid may be a non-naturally occurring or a naturally occurring amino acid. The skilled artisan will be readily able to determine regions which are generally evolutionary conserved amongst different species on the basis of sequence comparisons such as that shown in FIG. 2. The amino acids identical or similar between the species specifically mentioned above will furthermore be readily identifiable by the skilled artisan on the basis of the amino acid sequence comparisons depicted in FIGS. 16, 17, and 18 and the accompanying Tables (Tables 1, 2, and 3, respectively).

Preferably, the wild type residue of the modified AGR2 protein is replaced by an amino acid with different size and/or polarity, i.e., a non-conservative amino acid substitution, as defined below.

Also preferred is an AGR2 mutein wherein residue 137 of AGR2 according to SEQ ID NO:4 is replaced by an amino acid other than a large aliphatic, nonpolar amino acid, and preferably is replaced by an acidic amino acid and most preferably by a glutamic acid.

In one preferred embodiment a murine Agr2 mutein of the present invention has the amino acid sequence shown in SEQ ID NO:2.

In a further preferred embodiment a human AGR2 mutein of the present invention has the amino acid sequence shown in SEQ ID NO:30.

An “isolated” or “purified” polypeptide or protein, or a biologically active fragment thereof as described and claimed herein is substantially free of cellular material or other contaminating proteins from the cell or tissue source from which the polypeptide or protein is derived, or substantially free from chemical precursors or other chemicals when chemically synthesized. The language “substantially free of cellular material” includes preparations of AGR2 protein in which the protein is separated from cellular components of the cells from which the protein is isolated or in which it is recombinantly produced.

The invention furthermore encompasses mature mouse Agr2 or human AGR2 proteins, or their vertebrate orthologues, e.g., the specific orthologues referred to above, which comprise an amino acid or amino acid sequences corresponding to a mutation as defined above. As used herein, a “mature” form of a polypeptide or protein may arise from a post-translational modification. Such additional processes include, by way of non-limiting example, proteolytic cleavage, e.g., cleavage of a leader sequence, glycosylation, myristoylation or phosphorylation. In general, a mature polypeptide or protein according to the present invention may result from the operation of one of these processes, or a combination of any of them.

As mentioned above, when for example residue 137 of SEQ ID NO:3 is replaced by an amino acid with different size and/or polarity (excluding the wild type residue at this position), this is termed a non-conservative amino acid substitution. Non-conservative substitutions are defined as exchanges of an amino acid by another amino acid listed in a different group of the five standard amino acid groups shown below:
1. small aliphatic, nonpolar or slightly polar residues: Ala, Ser, Thr, (Pro), (Gly);
2. negatively charged residues and their amides: Asn, Asp, Glu, Gln;
3. positively charged residues: His, Arg, Lys;
4. large aliphatic, nonpolar residues: Met, Leu, Ile, Val, (Cys);
5. large aromatic residues: Phe, Tyr, Trp.

Conservative substitutions are defined as exchanges of an amino acid by another amino acid listed within the same group of the five standard amino acid groups shown above. Three residues are parenthesized because of their special role in protein architecture. Gly is the only residue without a side-chain and therefore imparts flexibility to the chain. Pro has an unusual geometry which tightly constrains the chain. Cys can participate in disulfide bonds.

The invention also provides novel chimeric or fusion proteins. As used herein, a novel “chimeric protein” or “fusion protein” comprises a novel AGR2 polypeptide linked to a non-AGR2 polypeptide (i.e., a polypeptide that does not comprise AGR2 or a fragment thereof).

In one embodiment, the fusion protein is a GST-AGR2 heavy chain fusion protein in which the AGR2 sequences are fused to the C-terminus of the GST (glutathione-S-transferase) sequences. Such fusion proteins can facilitate the purification of recombinant AGR2 polypeptides.

In yet another embodiment, the fusion protein is a AGR2-immunoglobulin fusion protein in which the AGR2 sequences are fused to sequences derived from a member of the immunoglobulin protein family, especially Fc region polypeptides. Also contemplated are fusions of AGR2 sequences (mutant proteins or fragments) fused to amino acid sequences that are commonly used to facilitate purification or labeling, e.g., polyhistidine tails (such as hexahistidine segments), FLAG tags, and streptavidin.

The amino acid sequences of the present invention may be made by using peptide synthesis techniques well known in the art, such as solid phase peptide synthesis (see, for example, Fields et al., “Principles and Practice of Solid Phase Synthesis” in SYNTHETIC PEPTIDES, A USERS GUIDE, Grant, G. A., Ed., W.H. Freeman Co. NY. 1992, Chap. 3 pp. 77-183; Barlos, K. and Gatos, D. “Convergent Peptide Synthesis” in FMOC SOLID PHASE PEPTIDE SYNTHESIS, Chan, W. C. and White, P. D. Eds., Oxford University Press, New York, 2000, Chap. 9: pp. 215-228) or by recombinant DNA manipulations and recombinant expression. Techniques for making substitution mutations at predetermined sites in DNA having known sequence are well known and include, for example, M13 mutagenesis. Manipulation of DNA sequences to produce variant proteins which manifests as substitutional, insertional or deletional variants are conveniently described, for example, in Sambrook et al. (1989), supra.

### Antibodies

A further aspect of the present invention are antibodies specifically recognizing an epitope in a mutein as described further below, wherein said epitope comprises the amino acid or the amino acid sequence in said protein which corresponds to the mutation described in connection with these muteins.

Also included in the invention are antibodies to fragments of mutein AGR2 polypeptides (including amino terminal fragments), as well as antibodies to fusion proteins containing AGR2 mutein polypeptides or fragments of AGR2 mutein polypeptides. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, e.g., polyclonal, monoclonal, chimeric, single chain, F_{ab}, F_{ab′} and F_{(ab′)2} fragments, and a F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An AGR2 polypeptide, i.e., wild type or mutant AGR2, as described herein, may be intended to serve as an antigen, or a portion or fragment thereof, and additionally can be used as an immunogen to generate antibodies that immunospecifically bind the antigen, using standard techniques for polyclonal and monoclonal antibody preparation. Antigenic peptide fragments of the antigen for use as immunogens includes, e.g., at least 7 amino acid residues of the amino acid sequence of the mutated region such as an amino acid sequence shown in SEQ ID NO:2, and in SEQ ID NO:30 or in SEQ ID NO:3 and SEQ ID NO:4, respectively, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of mutein or wild type AGR2 polypeptide that is located on the surface of the protein, e.g., a hydrophilic region. A hydrophobicity analysis of a mutein or wild type AGR2 polypeptide will indicate which regions of a mutein or wild type AGR2 protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., (Hopp and Woods, 1981; Kyte and Doolittle, 1982b; Kyte and Doolittle, 1982a). Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs, homologues or orthologues thereof. See, for example, ANTIBODIES: A LABORATORY MANUAL, Harlow and Lane (1988) Cold Spring Harbor Laboratory Press, Cold Spring Harbor, N.Y. Some of these antibodies are discussed below.

### Polyclonal Antibodies

For the production of polyclonal antibodies, various suitable host animals (e.g., rabbit, goat, mouse or other mammal) may be immunized by one or more injections with the protein of the invention, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor.

The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography.

### Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein (Kohler and Milstein, 1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, MONOCLONAL ANTIBODIES: PRINCIPLES AND PRACTICE, Academic Press, (1986) pp. 59-103. Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies ((Kozbor et al., 1984), Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Rodbard (Munson and Rodbard, 1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, 1994b) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

### Humanized Antibodies

The antibodies directed against the protein antigens of the invention can further comprise humanized antibodies or human antibodies. These is antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988b; Verhoeyen et al., 1988a; Riechmann et al., 1988a; Verhoeyen et al., 1988b), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues, which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988b; Riechmann et al., 1988a).

### Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique and the EBV hybridoma technique to produce human monoclonal antibodies (see Cole, et al., 1985 In: MONOCLONAL ANTIBODIES AND CANCER THERAPY, Alan R Liss, Inc., pp. 77-96). Human monoclonal antibodies may be utilized in the practice of the present invention and may be produced by using human hybridomas (Cote et al., 1983) or by transforming human B-cells with Epstein Barr Virus in vitro (see Cole, et al. (1985) In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, 1992; Marks et al., 1991a; Marks et al., 1991b). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in here: Fishwild et al., 1996b; Lonberg et al., 1994b; Lonberg and Huszar, 1995b; Marks et al., 1992; Morrison, 1994b; Neuberger, 1996b; Fishwild et al., 1996a; Lonberg et al., 1994a; Lonberg and Huszar, 1995a; Morrison, 1994a; Neuberger, 1996a.

Human antibodies may additionally be produced using transgenic non-human animals which are modified so as to produce fully human antibodies rather than the animal's endogenous antibodies in response to challenge by an antigen. See PCT publication WO94/02602. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker, and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

### F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (Huse et al., 1989) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

### Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, 1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829 and in Traunecker et al. (Traunecker et al., 1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al. (Suresh et al., 1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al. (Brennan et al., 1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al. (Shalaby et al., 1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers (Kostelny et al., 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology (Holliger et al., 1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported (Gruber et al., 1994).

Antibodies with more than two valencies are contemplated. For example, trispecific antibodies can be prepared (Tutt et al., 1991).

Exemplary bispecific antibodies can bind to two different epitopes, at least one of which originates in the protein antigen of the invention. Bispecific antibodies can also be used to direct various agents to cells, which express a particular antigen. These antibodies possess an antigen-binding arm and an arm, which binds an agent such as a radionuclide chelator (e.g., EOTUBE, DPTA, DOTA, or TETA).

### Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving cross-linking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody of the invention with respect to effector function, so as to enhance, e.g., the effectiveness of the antibody. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC) (Caron et al., 1992; Shopes, 1992a; Shopes, 1992b). Homodimeric antibodies with enhanced anti-tumor activity can also be prepared using heterobifunctional cross-linkers as described in Wolff et al. (Wolff et al., 1993). Alternatively, an antibody can be engineered that has dual Fc regions and can thereby have enhanced complement lysis and ADCC capabilities (Stevenson et al., 1989).

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹I, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described (Vitetta et al., 1983). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immonoconjugates according to the present invention are furthermore those comprising an antibody as described above conjugated to an imaging agent. Imaging agents suitable in this regard are, for example, again certain radioactive isotopes. Suitable in this regard are ¹⁸F, ⁶⁴Cu, ⁶⁷Ga, ⁶⁸Ga, ^{99m}Tc, ¹¹¹In, ¹²³I, ¹²⁵I, ¹³¹I, ¹⁶⁹Yb, ¹⁸⁶Re, and ²⁰¹Tl. Particularly preferred in this regard is ^{99m}Tc. The radioactive isotopes will suitably be conjugated to the antibody via a chelating group that is covalently attached to the antibody and is capable of chelating the radioactive isotope.

### Anticalins

Anticalins are engineered proteins with antibody-like binding functions derived from natural lipocalins as a scaffold. These small monomeric proteins of only about 150 to 190 amino acids may have certain competitive advantages over antibodies, e.g., an increased binding specificity and improved tissue penetration, for example in the case of solid tumors. The anticalins of the present invention preferably bind their ligands with high specificity and affinity in the nanomolar range, e.g., in the low nanomolar range with K(D) values ranging between 12 nM and 35 nM. The set of four loops of anticalins may be easily manipulated at the genetic level (Weiss and Lowmann, 2000; Skerra, 2001). A preferred anticalin according to the present invention specifically binds to the AGR2 muteins as described herein. Another preferred anticalin specifically binds to the wild type AGR2 protein, e.g., the AGR2 proteins according to SEQ ID NO:3 or SEQ ID NO:4.

Methods for producing aptamers specific for proteins and nucleic acids are known. See, e.g., U.S. Pat. No. 5,840,867, U.S. Pat. No. 5,756,291, and U.S. Pat. No. 5,582,981.

### Vectors and Cells Expressing AGR2 Protein

Another aspect of the invention pertains to vectors, preferably expression vectors, containing a nucleic acid encoding a AGR2 mutein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded circular DNA molecule into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”.

A host cell of the invention, such as a prokaryotic or eukaryotic host cell in culture, can be used to produce (i.e., express) AGR2 mutein. Accordingly, the invention further provides methods for producing AGR2 mutein using the host cells of the invention. In one embodiment, the method comprises culturing the host cell of invention (into which a recombinant expression vector encoding AGR2 mutein protein has been introduced) in a suitable medium such that AGR2 mutein is produced. In another embodiment, the method further comprises isolating AGR2 mutein from the medium or the host cell.

The host cells of the invention can also be used to produce non-human transgenic animals. For example, in one embodiment, a host cell of the invention is a fertilized oocyte or an embryonic stem cell into which AGR2 protein-coding sequences have been introduced. Such host cells can then be used to create non-human transgenic animals in which exogenous AGR2 sequences have been introduced into their genome or homologous recombinant animals in which endogenous AGR2 sequences have been altered. Such animals are useful for studying the function and/or activity of AGR2 protein and for identifying and/or evaluating modulators of AGR2 protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. Standard methods are known in the art that may be used in conjunction with the polynucleotides and of the invention and methods described herein to produce a transgenic animal expressing a modified AGR2 of the invention.

### Methods of Screening for Disease-Relevant AGR2 Alleles

In one aspect, the present invention relates to a method of identifying a protein or nucleic acid marker indicative of an increased risk of a human subject of developing a medical condition associated with an alteration in goblet cell function, said method comprising the step of analyzing a test sample derived from a human subject for the presence of a difference compared to a similar test sample if derived from a human subject unaffected by or known not to be at risk of developing said condition, wherein said difference is indicative of the presence of a mutation in an allele of the gene coding for the AGR2 protein according to SEQ ID NO:4, or in an allele of a gene coding for a protein which affects expression or function of said AGR2 protein.

The present invention furthermore relates to a method of identifying a protein or nucleic acid marker indicative of an association of a medical condition in a human subject which is associated with an alteration in goblet cell function with altered AGR2 expression or function, said method comprising the step of analyzing a test sample derived from a human subject for the presence of a difference compared to a similar test sample if derived from a human subject unaffected by or known not to be at risk of developing said condition, wherein said difference is indicative of the presence of a mutation in an allele of the gene coding for the AGR2 protein according to SEQ ID NO:4, or in an allele of a gene coding for a protein which affects expression or function of said AGR2 protein.

In the above methods, the test sample derived from a human subject may be directly obtained from said human subject. It may, however, also be a sample that has been obtained previously. Also included test samples according to the invention are, for example, cDNA preparations that have been prepared from mRNA obtained from a tissue sample from a human subject at an earlier stage. It may also be cloned or PCR-amplified DNA that originates from DNA contained in such tissue sample obtained at an earlier stage.

According to the claimed method, the test sample will be analyzed for a difference to a similar test sample derived from a human subject unaffected by or known not to be at risk of developing a medical condition associated with an alteration in goblet cell function. While the method may include actually deriving or directly obtaining a test sample from such a human subject for comparative purposes, the necessary information regarding the relevant structural features and properties of such similar test sample to be used for comparison will often already be available. Thus, it will often be sufficient for the purposes of the above methods of the invention to perform an analysis for a difference to a similar test sample as it would be observed if said similar test sample were in fact obtained from a human subject unaffected by or known not to be at risk of developing the above medical condition.

The test sample may be a nucleic acid sample, e.g., mRNA (or cDNA derived therefrom), or genomic DNA.

It may also be a protein sample.

The difference analyzed may be one relating to the expression level of said nucleic acid or protein. Alternatively, it may be analyzed whether there is a difference in terms of the nucleotide or the amino acid sequence level.

Accordingly, the above methods of the invention include embodiments wherein the step of analysis for differences between the test samples comprises the partial or complete determination of the sequence of the nucleic acid, or a PCR-amplified portion of the nucleic acid, of the test sample, and optionally also of the nucleic acid or at PCR-amplified portion of the nucleic acid of the similar test sample (or the similar test samples).

Suitable methods for the determination of partial or complete nucleic acid sequences, and thus, detection of the above-mentioned differences, are well known to the skilled artisan. They include, for example, Southern blotting, TGGE (temperature gradient gel electrophoresis), DGGE (denaturing gradient gel electrophoresis), SCCP (single chain conformation polymorphism) detection, and the like. High throughput sequence analysis methods such as those described by Kristensen et al. (Kristensen et al., BioTechniques 30 (2001), 318-332), which is incorporated herein by reference in its entirety, are likewise suitable, and hence, contemplated in connection with the present invention.

Suitable methods for the determination of partial or complete amino acid sequences are likewise well known, and include, for example, detection of particular epitopes within a protein sample via specific antibodies in dot blot, slot blot, or Western blot assays, or via ELISAs or RIAs, or partial amino acid sequence determination on a sequencer via Edman degradation. Also, high-throughput methods may again be employed.

A further aspect of the present invention is represented by a method for identifying a predisposition of a human subject for developing a medical condition associated with an alteration in goblet cell function, said method comprising the step of determining whether a test sample derived from said human subject indicates the presence of a mutation in an allele of the gene coding for the AGR2 protein according to SEQ ID NO:4 indicative of an increased risk of said human subject of developing said medical condition.

Also contemplated in connection with the present invention is a method for determining whether a medical condition in a human subject which is associated with an alteration in goblet cell function is associated with altered AGR2 expression or function, said method comprising the step of determining whether a test sample derived from said human subject indicates the presence of a mutation in an allele of the gene coding for the AGR2 protein according to SEQ ID NO:4 indicative of an altered AGR2 expression or function.

As in the case of the methods described above, while the methods described in the two preceding paragraphs may involve that the test sample is derived from the human subject directly, it may also be a sample that has been obtained previously. Furthermore, suitable test samples according to the invention are, for example, cDNA preparations that have been prepared from mRNA obtained from a tissue sample from a human subject at an earlier stage. It may also again be cloned or PCR-amplified DNA that originates from DNA contained in such tissue sample obtained at an earlier stage.

Again, the previously mentioned methods of determining partial or complete nucleic acid or amino acid sequences may be employed for the step of determining whether the test sample (which may be a nucleic acid or protein test sample as previously defined) indicates the presence of said mutation.

According to the above methods of identifying a predisposition in a human subject of developing a medical condition associated with an alteration in goblet cell function, or determining a potential association between such a medical condition with altered AGR2 expression or function, the test sample is analyzed for the presence of a mutation in an allele of the AGR2 gene which is either indicative of an increased risk of developing such a medical condition, or of an altered AGR2 expression or function. It will be appreciated that such mutations are inter alia those referred to herein in connection with the proteins and nucleic acids according to the invention, and that mutations of this kind may be readily identified, for example, by the in vitro assays or the animal model referred to in this regard. They may also be identified by any of the aforementioned methods of screening for disease-relevant AGR2 alleles.

### Pharmaceutical Compositions

The invention also includes pharmaceutical compositions containing agents that can modulate AGR2 activity, i.e., AGR2 mutein or wild type activity. These agents include biomolecules such as proteins, muteins, kinases, phosphatases, antibodies, antibody fragments, nucleic acids, ribozymes, anticalins, and aptamers as described herein, as well as pharmaceutical compositions containing antibodies to them (e.g., antibodies to muteins or wild-type proteins, anti-idotypic antibodies). In addition, the agent may also include chemical compounds, e.g., small molecule agonists or antagonists, that may affect AGR2 directly. Furthermore, the agents may be biomolecules and chemical compounds, such as the ones listed above or below, that affect the interaction between AGR2, i.e., AGR2 mutein or wild type protein, and its physiologic ligands, including the cell membrane.

The compositions are preferably suitable for internal use and include an effective amount of a pharmacologically active compound of the invention, alone or in combination, with one or more pharmaceutically acceptable carriers. The compounds are especially useful in that they have very low, if any toxicity.

The agents of this invention, and antibodies thereto, may be used in pharmaceutical compositions, when combined with a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of REMINGTON'S PHARMACEUTICAL SCIENCES (18th ed.), Alfonso R. Gennaro, ed. (Mack Publishing Co., Easton, Pa. 1990), a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J., U.S.A.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

For instance, for oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, and the like. Diluents, include, e.g., lactose, dextrose, sucrose, mannitol, sorbitol, cellulose and/or glycine.

Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, preferably about 1 to 50%, of the active ingredient.

The compounds of the invention can also be administered in such oral dosage forms as timed release and sustained release tablets or capsules, pills, powders, granules, elixers, tinctures, suspensions, syrups and emulsions.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in liquid prior to injection can be formulated. Injectable compositions are preferably aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances.

The compounds of the present invention can be administered in intravenous (both bolus and infusion), intraperitoneal, subcutaneous or intramuscular form, all using forms well known to those of ordinary skill in the pharmaceutical arts. Injectables can be prepared in conventional forms, either as liquid solutions or suspensions.

Parental injectable administration is generally used for subcutaneous, intramuscular or intravenous injections and infusions. Additionally, one approach for parenteral administration employs the implantation of a slow-release or sustained-released system, which assures that a constant level of dosage is maintained, according to U.S. Pat. No. 3,710,795, incorporated herein by reference.

Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defined above, may be also formulated as suppositories using for example, polyalkylene glycols, for example, propylene glycol, as the carrier. In some embodiments, suppositories are advantageously prepared from fatty emulsions or suspensions.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoactylates and cross-linked or amphipathic block copolymers of hydrogels.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, may be preferably provided in any form commonly used for oral dosage such as, for example, in scored tablets, time released capsules, liquid filled capsule, gels, powder or liquid forms. When provided in tablet or capsule form the dosage per unit may be varied according to well known techniques. For example, individual dosages may contain 0.5, 1.0, 2.5, 5.0, 10.0, 15.0, 25.0, 50.0, 100.0, 250.0, 500.0 and 1000.0 mg of active ingredient. It is well known that daily dosage of a medication, such as a medication of this invention, may involve between one to ten or even more individual tables per day.

The compounds comprised in the pharmaceutical compositions of the present invention may be administered in a single daily dose, or the total daily dosage may be administered in divided doses of two, three or four times daily.

Any of the above pharmaceutical compositions may contain 0.1-99%, preferably 1-70% (w/w or w/v) of the wild type AGR2 polypeptide, the proteins and fragments, or the antibodies and their various modified embodiments specifically described and claimed herein.

If desired, the pharmaceutical compositions can be provided with an adjuvant. Adjuvants are discussed above. In some embodiments, adjuvants can be used to increase the immunological response, depending on the host species, include Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Generally, animals are injected with antigen using several injections in a series, preferably including at least three booster injections.

### Gene Therapy

A further aspect of the present invention is a method of gene therapy comprising delivering to cells in a human subject suffering from or known to be at risk of developing a condition associated with an alteration in goblet cell function a DNA construct comprising a sequence of an allele of the AGR2 gene encoding the human AGR2 protein according to SEQ ID NO:4, or encoding a protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or a sequence of an allele of the AGR2 gene of a human subject unaffected by or known not to be at risk of developing said condition.

Also encompassed by the present invention is a method of gene therapy of the above kind wherein the DNA construct delivered to the cells of the human subject comprises a DNA sequence encoding the human AGR2 protein according to SEQ ID NO:4, or a human AGR2 protein encoded by the AGR2 gene of a human subject unaffected by or known not to be at risk of developing said condition, or a protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

Furthermore encompassed are methods wherein the DNA construct comprises a DNA sequence encoding an antisense nucleic acid according to the invention, or an antisense nucleic acid comprising a nucleotide sequence which is complementary to an mRNA encoded by the AGR2 gene of a human subject unaffected by or known not to be at risk of developing said condition.

Also encompassed are methods wherein the DNA construct comprises a DNA sequence encoding an siRNA as described and claimed herein.

Alternatively, the DNA construct may comprise a DNA encoding an aptamer specifically binding an AGR2 mutein or an AGR2 wild type protein as described herein.

In a further embodiment, the DNA construct may comprise a DNA sequence encoding an Agr2 mutein as described herein.

The use of a DNA construct as described above in a method of treating a human subject suffering from, or known to be at risk of developing a medical condition associated with an alteration in goblet cell function, said method comprising delivering said DNA construct to at least some of the cells of said human subject, preferably the subject's goblet cells, is also encompassed within the present invention.

### Method of Modulating AGR2 Activity and Corresponding Uses

A further aspect of the present invention is a method of preventing, treating, or ameliorating a medical condition in a human subject associated with an alteration in goblet cell function, said method comprising administering to said human subject a pharmaceutical composition comprising an agent capable of modulating AGR2 activity, i.e., AGR2 mutein or wild type activity, in said human subject. The medical condition associated with an alteration in goblet cell function as described above and throughout the present description may optionally be furthermore associated with an increase in proliferation of the glandular epithelium of the Brunner's gland.

The medical conditions may be associated with a decreased mucus production, e.g., dry eye syndrome, gastric disease, peptic ulcer, inflammatory bowel disease, in particular Crohn's disease or ulcerative colititis, or intestinal cancer.

Alternatively, the medical conditions may be associated with an increase in mucus production, e.g., asthma, chronic obstructive pulmonary disease (COPD), and cystic fibrosis.

The agent capable of modulating AGR2 activity may be one of the agents described and specifically claimed herein, e.g., one of the muteins, nucleic acids, e.g., nucleic acids encoding the muteins, antisense nucleic acids, siRNAs or aptamers directed against or specifically binding to the AGR2 muteins, antibodies, or small molecule agonists or antagonists of the AGR2 muteins or wild type AGR2 protein as described herein.

It will be appreciated that in situations where the above medical condition is caused by a mutation in one of the alleles of the AGR2 gene which leads to the expression of an AGR2 mutein with a reduced or abolished activity, antisense nucleic acids, siRNA molecules, aptamers, anticalins, or antibodies directed against said AGR2 mutein may be therapeutically useful. Alternatively, administration of an AGR2 mutein, or a nucleic acid coding therefore, which is characterized by an increased AGR2 activity, or administration of a nucleic acid capable of leading to an increased AGR2 expression (e.g., of the endogenous wild-type AGR2 or of a wild-type AGR2 encoded by said nucleic acid), may likewise be therapeutically useful in this regard.

In situations where an excess amount or activity of the endogenous AGR2 protein is the cause of the above medical condition, administration of an AGR2 mutein, or nucleic acid coding therefore, which is characterized by a decreased AGR2 activity, or administration of a nucleic acid capable of leading to a decreased AGR2 expression (e.g., of an endogenous mutated or a wild-type AGR2) may likewise be therapeutically useful in this regard.

It will be appreciated that agents relating to the wild type AGR2 protein will likewise be advantageously administered to a human subject suffering from a condition as mentioned above, e.g., in situations where a reduced amount or activity of the endogenous AGR2 is the cause of the above medical condition in the human subject. Accordingly, it will be appreciated that a wild type AGR2 protein may advantageously be administered to a human subject suffering from such a condition, or a protein having a certain amino acid sequence identity and showing the same, or essentially the same, biological activity in any of the in vitro assays mentioned herein before (or a fragment or fusion of such protein). Proteins suitable in this regard may be readily determined, e.g., with the help of these in vitro assays.

It will also be appreciated that in situations where an excess of endogenous AGR2 protein or activity is the cause of the medical condition in the human subject, antisense nucleic acids, siRNAs molecules, aptamers, anticalins, or antibodies against said AGR2 wild type protein, may be therapeutically used.

It will be understood that the skilled person may use the in vitro assays as described herein in order to identify the activity of a given AGR2 mutein or the effect of an agent relating to such an AGR2 mutein or AGR2 wild type protein. Based on this information, the skilled person will be readily able to choose and identify the appropriate agent in connection with the disease situation to be treated.

### Assays and Diagnostics

The animals of the present invention present a phenotype whose characteristics are representative of many symptoms associated with disorders of altered mucus production and/or function, therefore making the animal model of the present invention a particularly suitable model for the study of these diseases including asthma, chronic obstructive pulmonary disease (COPD), cystic fibrosis, dry eye syndrome, gastric disease, peptic ulcer, inflammatory bowel disease and malignancies like colorectal cancer.

The animals of the present invention can also be used to identify early diagnostic markers for diseases associated with AGR2 deficiency. The term deficiency refers to an alteration of protein function in both positive (=gain of function) and negative (=loss of function) ways. Surrogate markers, including but not limited to ribonucleic acids or proteins, can be identified by performing procedures of proteomics or gene expression analysis known in the art. For example procedures of proteomics analysis include, but are not restricted to, ELISA, 2D-gel, protein microarrays or mass spectrophotometric analysis of any organ or tissue samples, such as blood samples, or derivatives thereof, preferably plasma, at different age or stage of AGR2 activity deficiency or activity increase associated disease development, or symptom thereof. As a further example, gene expression analysis procedures include, but are not restricted to, differential display, cDNA microarrays, analysis of quality and quantity of ribonucleic acids species from any organ or tissue samples, such as blood samples, or derivatives thereof, at different age or stage of development of AGR2 activity deficiency associated disease, or symptom thereof.

The animal model of the present invention can be used to monitor the activity of agents useful in the prevention or treatment of the above-mentioned diseases and disorders. The agent to be tested can be administered to an animal of the present invention and various phenotypic parameters can be measured or monitored. In a further embodiment the animals of the invention may be used to test therapeutics against any disorders or symptoms that have been shown to be associated with AGR2 deficiency or over-expression.

The animals of the present invention can also be used as test model systems for materials, including but not restricted to chemicals and peptides, particularly medical drugs, suspected of promoting or aggravating the above-described diseases associated with AGR2 deficiency. For example, the material can be tested by exposing the animal of the present invention to different time, doses and/or combinations of such materials and by monitoring the effects on the phenotype of the animal of the present invention, including but not restricted to change of goblet cell function, namely proper mucin production. Furthermore, the animals of the present invention may be used for the dissection of the molecular mechanisms of the AGR2 pathway, that is for the identification of receptors or downstream genes or proteins thereof regulated by AGR2 activity and deregulated in AGR2 activity deficiency or activity increase associated disorders. For example, this can be done by performing differential proteomics analysis, using techniques including but not restricted to 2D gel analysis, protein chip microarrays or mass spectrophotometry, on tissues of the animal of the present invention which express AGR2 and which respond to AGR2 stimuli.

An exemplary method for detecting the presence or absence of AGR2 mutein in a biological sample involves obtaining a biological sample from a test subject and contacting the biological sample with a compound or an agent capable of detecting AGR2 protein or nucleic acid (e.g., mRNA, genomic DNA) that encodes AGR2 mutein such that the presence of AGR2 is detected in the biological sample. An agent for detecting AGR2 mutein mRNA or genomic DNA is a labeled nucleic acid probe capable of hybridizing to AGR2 mutein mRNA or genomic DNA.

The diagnostic methods described herein can furthermore be utilized to identify subjects having or at risk of developing a disease or disorder associated with aberrant AGR2 expression or activity. For example, the assays described herein, such as the preceding diagnostic assays or the following assays, can be utilized to identify a subject having or at risk of developing a disorder associated with AGR2 protein, nucleic acid expression or activity. Alternatively, the prognostic assays can be utilized to identify a subject having or at risk for developing a disease or disorder. Thus, the invention provides a method for identifying a disease or disorder associated with aberrant AGR2 expression or activity in which a test sample is obtained from a subject and AGR2 protein or nucleic acid (e.g., mRNA, genomic DNA) is detected, wherein the presence of AGR2 protein or nucleic acid is diagnostic for a subject having or at risk of developing a disease or disorder associated with aberrant AGR2 expression or activity. As used herein throughout the entire specification, a “test sample” refers to a biological sample obtained from a subject of interest. For example, a test sample can be a biological fluid (e.g., blood, plasma; serum), cell sample, or tissue sample.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant AGR2 expression or activity. For example, such methods can be used to determine whether a subject can be effectively treated with an agent for a disorder.

Agents, or modulators that have a stimulatory or inhibitory effect on AGR2 activity (e.g., AGR2 gene expression), as identified by a screening assay described herein can be administered to individuals to treat (prophylactically or therapeutically) AGR2-mediated disorders. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of AGR2 protein, expression of AGR2 nucleic acid, or mutation content of AGR2 genes in an individual can be determined to thereby select appropriate agent(s) for therapeutic or prophylactic treatment of the individual.

The present invention also provides a diagnostic method for AGR2 activity deficiency or activity increase. Patients' peptide material, particularly that in or from blood, serum or plasma, is subjected to analysis for one or more of the amino acid sequences of the present invention. The peptide material may be analyzed directly or after extraction, isolation and/or purification by standard methods.

In one embodiment of the invention, the diagnostic method comprises the identification of the modified AGR2, whereby the modification is associated with the replacement of an amino acid at a position corresponding to position 137 in the amino acid sequence shown in SEQ ID NO:4. The diagnostic methods of the invention also include those employing detection of the modified AGR2 by its activity in competing with and blocking the action of native AGR2. Methods of identifying the modified AGR2 include any methods known in the art which are able to identify altered conformational properties of the amino acid sequence of the present invention compared to those of the wild type AGR2. These include, without limitation, the specific recognition of the modified protein by other proteins, particularly antibodies; individual or combined patterns of amino acid sequence digestion by known proteases or chemicals. In an additional, similar embodiment, the method exploits the failure of another protein to recognize the modified protein, examples being antibodies directed to an epitope of wild type AGR2 that incorporates residue 137 of SEQ ID NO:4, and AGR2 receptors in which this portion of the molecular surface of wild type AGR2 is recognized or involved in AGR2.

In a further embodiment of the present invention, the principle of the diagnostic method is the detection of a nucleic acid sequence encoding the modified AGR2 of the invention. This includes, but is not restricted to any methods known in the art using nucleic acid hybridizing properties, such as Polymerase Chain Reaction (PCR), Northern blot, Southern blot, nucleic acid (genomic DNA, cDNA, mRNA, synthetic oligonucleotides) standard methods employing microarrays, and patterns of nucleic acid digestion by known restriction enzymes.

The invention will be further described in the following examples, which do not limit the scope of the invention described in the claims. The following examples are offered for illustrative purposes only, and are not intended to limit the scope of the present invention in any way.

### Other features and advantages of the invention will be apparent from the following examples.

### EXAMPLE 1

### ENU (Ethyl-nitroso-urea) Treatment to Produce Mutagenized Animals

To produce mutants, a C3HeB/FeJ male mouse (The Jackson Laboratory, Bar Harbor Me., U.S.A.) was injected intraperitoneally three times (weekly intervals between 8-10 weeks of age) with ethyl-nitroso-urea (ENU) (Serva Electrophoresis GmbH, Heidelberg, Germany) at a dosage of 90 mg/kg body weight. The injected male mouse was regularly mated to wild type C3HeB/FeJ female partners fifty days after the last injection. The resultant F1 progeny (up to 100 offspring) were then analyzed for dominant phenotypes.

### Generation of F3 Progeny—Breeding Scheme

F3 progeny are generated using the breeding scheme shown in FIG. 3A. AU breeding partners were older than 8 weeks); preferably females were between 8-12 weeks of age and males were between 8-16 weeks of age.

### Production of F1-Animals (db1)

Each ENU-male produced as described above is used to generate more than 30 male and 30 female pups, which were interbred as described below.

### Production of F2-Animals (rf1)

Each week, 20 matings are set up as follows: (1 male F1(db1)×1 female F1(db1) to produce 20 pedigrees. The animals of one breeding pair are pups of different ENU-animals (mating type: rf1).

### Production of F3-Animals (rbs)

8 weeks rf1 animals are mated in single F2 (1 male)×F2 (1 female)—breeding per pedigree (mating type: rbs). From each rbs-breeding, at least 15 offspring are produced. Rf1-females are kept until the youngest rbs animals have been screened (age=160 days). Rf1-males are sacrificed and frozen after the number of 15 offspring has been reached. F3 animals are analyzed in the primary screen.

We performed a series of tests on F3 animals as a primary screen to identify relevant phenotypes. For this invention, observation of diarrhea and results of a routine histological examination provided information to identify an aberrant phenotype within the F3 population.

### EXAMPLE 2

### Physiological Characteristics of the Mutant Animals

The macroscopic evaluation indicates that 100% of the homozygous MTZ offspring in a C3H inbred background developed a macroscopically visible diarrhea and a thriving deficit. Thriving deficit is manifested in reduced weight in combination with reduced body length, when compared to wild type littermates.

### EXAMPLE 3

### Necroscopy and Organ Histology of the Mutant Animals

The visible diarrhea and the thriving deficit led to the subsequent investigation of the intestinal organs of the MTZ mouse.

For example, a histological examination of hematoxilin/eosin stained (FIG. 11), or of lectin stained (FIG. 13) colon wall sections from MTZ affected animals depict a strong reduction in pre-mucin storing granules in goblet cells, resulting in reduced mucus secretion and secondary inflammatory infiltrations in the colon mucosal epithelium and submucosa (marked by an asterisk in FIG. 12). Additionally, microerosion of colonic mucosa is detectable (marked by an arrow in FIG. 12). Paneth cells and enterochromaffin cells are not affected.

The observation that absence of normal AGR2 protein leads to dysfunctional goblet cells is be to extended to other mucosal organs expressing murine Agr2 mRNA, such as the eye, nose, trachea, lung, esophagus, salivary gland, stomach, intestine, rectum, thymus, testis, epididymis, uterus and placenta, as determined by RT-PCR, and as described in Example 6, and as shown in FIG. 6. Northern analysis of human mRNA confirmed the expression of Agr2 mRNA in all goblet cell carrying tissues and organs of the gastrointestinal tract, of the respiratory tract and in prostate and cervix, as described in Example 8, and as shown in FIG. 8.

In addition to the goblet cell phenotype described for MTZ colon, affected mice display a dilated Brunner's gland with increased proliferating glandular epithelium. Duodenal epithelium closely located to the Brunner's gland is characterized by loss of goblet cells, proliferated epithelium and signs of slight inflammation, as shown in FIG. 14. AGR2 mRNA expression in Brunner's glands was detected by RNA in situ-hybridization technique.

### EXAMPLE 4

### Mapping and Cloning of the Mutation in the Mutant Animals of the Present Invention

### 1. Generation of F5 Outcross Mice for Subsequent Chromosome Mapping

F5 progeny are generated according to the scheme illustrated in FIG. 3B—this entails breeding a phenotypically identified F3 mutant with C57Bl/6 mice for generation of F4 outcross mice. F4 progeny are then intercrossed to produce an F5 generation. The F5 generation is phenotyped according to the previously described parameters. Starting with two F3 animals of the MTZ pedigree we generated 40 F4 animals (22 males, 18 females) and 236 F5 animals (115 males, 121 females). The F5 outcross mice were used to locate the MTZ phenotype causing ENU mutation in the mouse genome.

### 2. DNA Isolation from Rodent Tails

Mouse genomic DNA was purified from 1 cm long pieces of mice tail by using the “DNeasy 96 Tissue Kit” (Qiagen, Hilden, Germany) according to the manufacturer's protocol.

### 3. Macromapping

In F5 outcross mice allele frequencies of C57Bl/6 versus C3H alleles are 1:1 in average, following Mendelian rules of inheritance. Arrangement in groups of phenotypic positive and phenotypic negative mice alters this ratio only at marker positions in the vicinity of the phenotype causing mutation driving it towards 0:1 in the phenotypic positive group and 1:0 in the phenotypic negative group. Allele frequency analysis of distributed genome covering markers (e.g., SSLP, SNP) in a group of phenotype positive F5 outcross mice indicate the site of the mutation as values for the C3H:C57Bl/6: ratio increase above 3.

For the MTZ mice we analyzed for a chromosomal locus with increased allele frequency for single nucleotide polymorphisms (SNPs) representing the C3H strain. Markers in this analysis are 90 SNPs polymorphic between C3H and C57Bl/6 strains, equally distributed over the 19 autosomal mouse chromosomes. Analysis was done in two steps at pooled tail DNA samples of 14 F5 outcross mice positive for the MTZ phenotype. First: competitive PCR, followed by second: SNP allele frequency measurement from the PCR product mix by Pyrosequencing technology (PSQ 96 system; http://www.pyrosequencing.com/pages/applications.html).

Pooled tail DNA (1 ml 10 μg/ml: 10 μg/14 mice=0.71 μg/mouse (concentration roughly judged and adjusted by agarose gel comparison to standard), pooled, ad 1 ml) was distributed in a 96-well plate with predeposited SNP marker PCR primers (one SNP/well). A standard PCR reaction was performed (50 μl vol.). One of both SNP primers was biotinylated, which is necessary for the subsequent single strand PCR product purification in the Pyrosequencing procedure. Purification of a single stranded (ss) PCR product and short range sequencing the SNP positions on the ss PCR product was performed according to the instructions supplied with the Pyrosequencing kit (PSQ 96 SNP Reagent Kit, 5×96). The resulting peaks at the polymorphic bp positions of the SNP sequence correlate to the amount this allele had in the original DNA pool and were exported from the PSQ 96 databank and processed into an Excel macro.

The Excel macro calculated the C3H/BL6-peakhight ratio at every SNP position according to the formula: (peakhight^{C3H}/peakhight^{BL6})/constant^{individualSNP}. Constant^{individualSNP} serves to improve C3H/BL6-peakhight ratio comparability among different SNP positions and is an average value for peakhight^{C3H}/peakhight^{BL6} of a heterozygous C3H/C57Bl/6 mouse (F1 outcross mouse). This value was determined experimentally afore for every individual SNP from nine (triplicates on three days) measurements and is expected to be close to 1 in theory but often differs from 1 in practice. Finally the Excel macro delivered a graphical output from the calculated B16/C3H-peakhight ratios (FIG. 4) in which regions with values above 3 indicate the chromosomal position of the mutation.

The output for MTZ phenotype positive DNA pool analysis showed high values above 3 at chromosome 12 and assigned the mutation to chromosome 12, 0-30 cM.

### 4. Fine Mapping

The initial mapping was confirmed on single mouse level haplotype analysis of a total of 236 F5 outcross MTZ mice using microsatellite markers located in the critical region on chromosome 12. Successively the candidate region mapping was refined, based on mice that carry chromosomal break points in the respective region. Finally the analysis narrowed the location of the mutation to an interval of approximately 25.7 Mbp between the SNP marker Idb2 (SEQ ID No:11, primer SEQ ID No:12, 13) and D12Mit64 (SEQ ID No:14, primer SEQ ID No:15, 16). This was evident since MTZ mouse #764 (phenotype positive) excluded the region proximal of Idb2, while MTZ mice #799 and #899 (both phenotype positive) excluded the region distal of D12Mit64 (FIG. 5). This results into the conclusion that a gene located entirely or partially between these markers could contain the mutation.

The genomic interval between markers Idb2 and D12Mit64 was scanned for genes by a detailed analysis of public mouse and human genome databases. Several annotated mouse genes were recorded within this region. Of these, the identified AGR2 gene was considered one of the most relevant candidate genes to search for the mutation, as it was known to be expressed in goblet cells.

### 5. PCR Amplification and Sequencing of Mouse Agr2 Gene

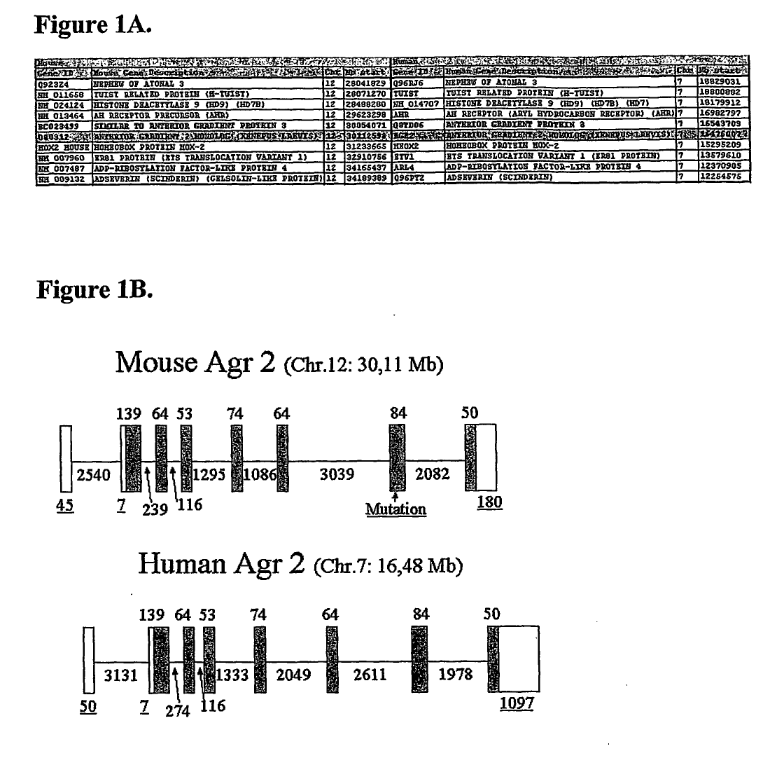

The genomic structure, precise location of AGR2 exons and a putative full length cDNA (SEQ ID No:6), containing the open reading frame coding for the AGR2 protein (SEQ ID No:3), an poly adenylation signal, and a polyA stretch was deduced from a public available mouse Agr2 cDNA sequence (Genbank accession number NM_{—}011783) and from genomic mouse DNA data (Ensemble, February 2002 freeze of the mouse assembly). The same was done for human AGR2 (Genbank accession number NM_{—}006408). For mouse Agr2, 8 exons could be defined (see FIG. 1B) that very closely resemble the human AGR2 gene in respect to size, sequence, genomic context and chromosomal exon distribution, suggesting evolutionary conserved functions for mouse and human AGR2 (see FIG. 1).

Genomic DNA fragments of AGR2 gene were obtained by PCR using BioTherm-DNA-polymerase (GeneCraft, Germany) according to the manufacturer's protocol. Oligonucleotide primers were designed using a publicly available primer design program (Primer 3, www.genome.wo.mitedu) to generate a series of oligonucleotide primers specific for AGR2 exons. Primers used for amplification are shown in SEQ ID NO:17 to SEQ ID NO:28. (Primers SEQ ID No:17 and 18 were used to amplify exon 2, SEQ ID NO:19 and 20 were used to amplify exon 3+4, SEQ ID NO:21 and 22 were used to amplify exon 5, SEQ ID NO:23 and 24 were used to amplify exon 6, SEQ ID NO:25 and 26 were used to amplify exon 7, SEQ ID NO:27 and 28 were used to amplify exon 8, exon 1 was not sequenced, since it is a noncoding exon). PCR amplified products were purified using the QIAquick PCR Purification Kit (Qiagen, Hilden, Germany) according to the manufacturer's protocol. PCR products were sequenced using forward/reverse PCR primers and the “Big Dye” thermal cycle sequencing Kit (ABI PRISM, Applied Biosystems, Foster City, Calif., U.S.A.). The reaction products were analyzed on an ABI 3700 DNA sequencing device.

### 6. Sequence Analysis

The sequences were edited manually and different sequence fragments were assembled into one contiguous sequence the software Sequencer version 4.0.5. (Gene Codes Corp., Ann Arbor Mich., U.S.A.). We sequenced the AGR2 gene in MTZ phenotype positive homozygous F2 outcross mice as well as heterozygous mice. In both cases, C3H and C57Bl/6 mice sequences were used as controls. The sequencing results showed that exons 2-6 and exon 8 were free of any mutation. However, a single bp exchange in exon 7 changing the underlined T in sequence ATCCCTGACGGTGAGGGCAGAC (see SEQ ID NO:6) to A (see SEQ ID NO: 1), resulting in an A/T double peak in the heterozygous mice and a pure A in the homozygous MTZ mice. The mutation was confirmed in all MTZ phenotype positive mice tested. Sequencing the coding region from other genes in the candidate region showed that those were free of any additional mutation.

As a consequence of the identified mutation the codon GTG is changed to GAG and the mutated AGR2 protein carries a charged glutamic acid (E) in position 137 instead of the non polar valin (V) in the wild type (non mutated) protein.

### EXAMPLE 5

### Method for Production of the Mutant Animals of the Present Invention by Gene Targeting Technology

The construction of a recombinant targeting vector to insert a point mutation in exon 7 of the mouse Agr2 gene may be performed according to well known techniques. For example the Lambda-KO-Sfi system of Nehls and Wattler, WO 01/75127.

### 1. Vector Construction

In a first step, a 1.5 kbp genomic DNA fragment is PCR amplified, representing the left arm of homology of the targeting vector to be constructed. After subsequent subcloning of the PCR fragment into a plasmid vector, i.e. pCR 2.1-TOPO (K4500-01, Invitrogen, Carlsbad, Calif., USA), according to the manufacturer's instructions, plasmid DNA, bearing the correct AGR2 insert is subject to site-directed mutagenesis, using a QuickChange Site-Directed Mutagenesis Kit (200518, Stratagene, La Jolla, Calif., USA), as outlined in the manufacturer's instructions. In brief, the plasmid vector (parental DNA template) and two oligonucleotide primers, each primer complementary to opposite strands of the vector insert and containing the desired point mutation (exon 7, position 462 of AGR2 cDNA), are denatured and subject to PCR amplification with a proof-reading DNA polymerase (Pfu Turbo), provided in the kit. Using the non-strand displacing action of Pfu Turbo DNA polymerase, mutagenic primers are incorporated and extended, resulting in nicked circular DNA strands. In a restriction digest with DpnI, only the methylated parental DNA template is susceptible to DpnI digestion. After transformation in XL1-Blue supercompetent cells, provided with the kit, nicks in the mutated (point mutation) plasmid DNA are repaired. Mutation positive colonies are selected and plasmid DNA is isolated, according to the manufacturer's instructions (Stratagene, La Jolla, Calif., USA).

Plasmid DNA, bearing the point mutation in exon 7, as described in the present invention, is subject to PCR amplification with primers, bearing SfiC and SfiA sequence overhangs, respectively, as described in the published patent application WO 01/75127. The PCR fragment, representing the left arm of homology is further processed, as described in the aforementioned patent application. The vector described in WO 01/5127, includes a linear lambda vector (lambda-KO-Sfi) that comprises a stuffer fragment, an E. coli origin of replication, an antibiotic resistance gene for bacteria selection, two negative selection markers suitable for use in mammalian cells, and LoxP sequences for cre-recombinase mediated conversion of linear lambda phages into high copy plasmids. In a final lambda targeting vector, the stuffer fragment is replaced by Sfi A,B,C,D ligation of the left arm of homology (bearing the AGR2 point mutation in exon 7), an ES cell selection cassette, and a right arm of homology, as described in the aforementioned patent application. In-vitro packaging of the ligation products, plating of a phage library, plasmid conversion, and DNA isolation of the homologous recombination plasmid vector is performed according to standard procedures, known by persons skilled in the art.

### 2. ES Cell Transformation and Mice Production.

Targeting vectors containing the point mutation are used for mouse ES cell transformation and to producing chimeric mice by blastocyst injection and transfer using standard methodology, well known in the art. The chimeras are bred to wild type mice to determine germline transmission. Heterozygotes and subsequently homozygotes are generated according to well known techniques.

### EXAMPLE 6

### Expression of Murine AGR2

To identify the cellular RNA expression pattern of the murine AGR2 gene, reverse transcribed polymerase chain reaction (RT-PCR) was employed. A tissue cDNA panel of 48 different tissues or developmental stages of the mouse was used, comprising the following tissues: total brain, cerebrum, cerebrum left hemisphere, cerebrum right hemisphere, cerebellum, medulla oblongata, medulla spinalis, thyreoidea/trachea, olfactory lobes, lung, tongue, esophagus, salivary gland, stomach, pituitary gland, pancreas, small intestine, large intestine, eye, appendix, nose epithelium, rectum, trachea, thymus, heart, uterus, mesenterium, placenta, gall bladder, sternum, liver, bone marrow, spleen, whole blood, kidney, skin, adrenal gland, adipose tissue, bladder, skeletal muscle, testis, Es-cells, epididymis, prostate, embryo d 5,5, embryo d 9,5, embryo d 13,5 head, embryo d 13,5 body, embryo d 18,5 head, embryo d 18,5 body, embryo d 10-12 (Ambion), cDNA pool, plus a negative (water) control:

The primers used are the following: mAgr2-7 5′-CAGACCCTTGATGGTCATTC; SEQ ID NO:7, mAgr2-2 5′-GTCTCCTGACCCGGTGCGCAG; SEQ ID NO:8. The PCR product of 349 bp in length represents a PCR product specific for mouse Agr2, as verified by sequence analysis. Expression of mouse AGR2 was identified in the following cells and organs: medulla oblongata, eye, nose epithelium, trachea, thyreoidea, lung, esophagus, salivary gland, stomach, small intestine, large intestine, appendix, rectum, gall bladder, testis, epididymis, uterus, placenta, embryo at day 5.5 and embryo at day 13.5, as seen in FIG. 6.

### EXAMPLE 7

### Expression of Human AGR2

To identify the cellular RNA expression pattern of the human AGR2 gene, reverse transcribed polymerase chain reaction (RT-PCR) was employed. A tissue cDNA panel of 29 different tissues from human was used, comprising the following tissues: total brain, cerebellum, trachea, lung, esophagus, stomach, salivary gland, pancreas, colon, rectum, thymus, heart, pericardium, liver, fetal liver, spleen, kidney, adrenal gland, bladder, uterus, cervix, placenta, breast, mammary gland, testis, prostate, skin, adipose tissue, skeletal muscle. The primers used are the following: hAGR2-1 5′-GAACCTGCAGATACAGCTCTG; (SEQ ID NO:9) hAGR2-4 5′-CACACTAGCCAGTCTTCTCAC; (SEQ ID NO:10). The PCR product 170 bp in length represents the PCR product specific for human AGR2, as verified by sequence analysis. Strong expression of human AGR2 was identified in the following tissues: trachea, stomach, salivary gland, colon, rectum, kidney, uterus, cervix, mammary gland, prostate, as seen in FIG. 7.

### The tissue specific expression profile of both genes, mouse AGR2 and human AGHR2, is very similar.

### EXAMPLE 8

### Tissue-Specific Expression of human Agr2 mRNA, Analyzed by Northern Hybridization

Northern hybridization of polyA⁺ RNAs from several human tissues was carried out using a human AGR2 specific DNA probe. The probe was generated by radiolabeling a purified and sequence-verified PCR product generated by using primers hAgr2-3 (SEQ ID NO:31) and hAgr24 (SEQ ID NO:32), comprising the open reading frame of AGR2. The probe is 532 bp in length (see SEQ ID NO:33). Commercially available Multiple Tissue Northern Blots (4 different MIN blots (MTN1, MTN2, MTN3, MTN4) of BioChain Institute, Hayward Calif., USA) each containing 3 micrograms of poly A⁺ RNA per lane; Human Digestive System 12 lane MTN (MTN12) blot by Clontech/Becton Dickinson, San Jose, USA, each lane containing 3 micrograms of poly A⁺ RNA) were hybridized, following the manufacturer's instructions. These blots are optimized to give best resolution in the 1.0-4.0 kb range, and marker RNAs of 9.5, 7.5, 4.4, 2.4, 1.35 and 0.24 kb were run as reference. Membranes were pre-hybridized for 30 minutes and hybridized overnight at 68° C. in ExpressHyb hybridization solution (Clontech Laboratories, Palo Alto Calif., USA) as per the manufacturer's instructions. The DNA probe used was labeled with [α³²P] dCTP using a random primer labeling kit (Megaprime DNA labeling system; Amersham Pharmacia Biotech, Piscataway N.J., USA) and had a specific activity of 1×10⁹ dpm/μg. The blots were washed several times in 2×SSC, 0.05% SDS for 30-40 minutes at room temperature, and were then washed in 0.1×SSC, 0.1% SDS for 40 minutes at 50° C. (see Sambrook et al., 1989, “Molecular Cloning, A Laboratory Manual”, Cold Spring Harbor Press, New York, USA). The blots were covered with standard domestic plastic wrap and exposed to X-ray film at −70° C. with two intensifying screens for 18 hours.

The tissues represented in the Clontech/Becton-Dickinson and in the BioChain Institute Multiple Tissue Northern Blots are as follows:

| MTN 12 | MTN 1 | MTN 2 | MTN 3 | MTN 4 |
|---|---|---|---|---|
| esophagus | stomach | brain | heart | uterus |
| stomach | jejunum | kidney | brain | cervix |
| duodenum | ileum | spleen | liver | ovary |
| ileocecum | colon | intestine | pancreas | testis |
| ileum | rectum | uterus | skeletal muscle | prostate |
| jejunum | lung | cervix | lung | lung |
| ascending colon | | placenta | | |
| descending colon | | lung | | |
| transverse colon | | | | |
| caecum | | | | |
| rectum | | | | |
| liver | | | | |

The results of this experiment indicate that human AGR2 mRNA is strongly expressed in stomach, duodenum, ileocecum, ileum, descending colon, transverse colon, caecum, and rectum. Weaker expression is detected in lung, cervix, and prostate (see FIG. 8). The usage of two different polyadenylation signals leads to AGR2 transcripts of 950 nucleotides and of 1800 nucleotides in lengths.

### EXAMPLE 9

### Characteristics of Human and Mouse AGR2 Protein and Tissue Specific Expression

The human orthologue of the mouse Agr2 protein, human AGR2 protein, has a length of 175 amino acid residues (in comparison to 175 amino acid residues for the corresponding mouse protein). FIG. 2 represents an amino acid alignment of mouse Agr2 and human AGR2, indicating an amino acid identity of 91%, indicating that these are orthologues.

Murine Agr2 protein was detected in goblet cells, using an anti-murine Agr2 antiserum, as described in Example 11, and as shown in FIG. 10. Goblet cell specificity was confirmed with an anti-TFF3 antibody (kindly provided by W. Hoffmann, Universitätsklinikum Magdeburg, Germany). In situ-hybridization confirmed Agr2 protein expression in Brunner's glands (data not shown).

### EXAMPLE 10

### Cloning of Mouse and Human AGR2 into Expression Vectors

To express wild type or mutant AGR2 in bacteria or eukaryotic cells, the cDNA can be cloned into a expression vector using standard cloning and transfection techniques, as described, for instance, in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993. A preferred method is the cDNA subcloning into expression vectors of the Gateway cloning and expression system (Invitrogen, California, USA), according to the manufacturer's instructions.

Purification of recombinant AGR2 from host cells can be performed using standard methods well-known to those skilled in the art. For standard references, see above.

### EXAMPLE 11

### Method for the Production of Antibodies Specific for AGR2 Epitopes

The production of antibodies specific for AGR2 was performed according to well known techniques, as described for example herein or in Paul Suhir, Antibody engineering Protocols, Humana Press, 1995 and William C. Davis (ed), Monoclonal antibody production, Humana Press 1995.

### 1. Preparation of Antigens

To obtain antigen for the immunization of animals, recombinant AGR2 proteins or fragments thereof may be expressed in pro- or eukaryotic cells and purified from the cell lysates according to standard techniques as described for example in Joseph Sambrook et al., Molecular Cloning: A Laboratory Manual, Cold Spring Harbor Laboratory Press; 3^{rd} ed. 2001), and as described in Example 10. Alternatively, specific peptides with approximately up to ˜60, preferably 15 to 25 residues with a sequence identical to parts of AGR2, were synthesized and coupled to keyhole limpet hemocyanin (KLH) or bovine serum albumin (BSA) via an additional cysteine at the C- or N-terminus as described in Schnölzer et al. (1992). Peptides for immunizations can be derived from any part of the amino acid sequence of AGR2, preferably from regions with high probability for localization on the surface of the protein (as predicted for example with the sequence analysis tools of The European Molecular Biology Open Software Suite) and with low sequence homology to other known proteins, preferably the peptide TVKSGAKKDPKDSRPKLPQ (SEQ ID NO:34)

### 2. Immunization

For the production of antibodies in animals, the synthetic peptides coupled to a carrier protein or the purified recombinant protein were injected subcutaneously into an animal. For a mouse or rabbit, 100 to 200 μg of antigen were used. Antigen were dissolved in a suitable adjuvant, preferably Complete Freund's Adjuvant (Sigma, St Louis, Mo., USA) for the initial injection, and Freund's Incomplete Adjuvant (Sigma) for all subsequent injections, to a final volume of about 200 μl per animal.

Booster injections were given after several weeks, preferably 5, 9 and 13 weeks after the first injection. Shortly after the fourth injection, preferably after ten days, the animals were anesthetized and killed by heart punctation. Sera were separated.

### EXAMPLE 12

### Western Blot Analysis. AGR2 is a Secreted Protein Released from Cultured Colon Cancer Cells

Western blot analysis was performed as described in Ausubel et al. (eds.), CURRENT PROTOCOLS IN MOLECULAR BIOLOGY, John Wiley & Sons, New York, N.Y., 1993. AGR2 protein was detected using the anti-murine AGR2 antiserum, as described in Example 11. The human colon cancer cell lines Caco-2 (ATCC No. HTB-37), HT-29 (ATTC No. HTB-38), and LS 174T (ATTC No. CL-188) endogenously express human AGR2 protein. In contrast the simian fibroblastoid cell line COS-7 (ATTC No. CRL-1651) does not express detectable amounts of AGR2 protein. (See FIG. 20, IP (immunoprecipitated) cell pellet.) In the present example, 1×10⁷ cells were lysed in 1 ml detergent lysis buffer containing 1% NP-40, 25 mM Tris pH 7.5, 150 mM NaCl and 5 mM EDTA. Protein concentrations were determined and amounts of lysate corresponding to 30 μg of total protein were resolved by SDS-PAGE. After blotting on nitrocellulose membranes, AGR2 protein was detected using an AGR2 specific rabbit antiserum (1:1000 fold dilution in TBST) and a secondary, peroxidase-coupled anti-rabbit IgG reagent. Visualization was achieved by chemiluminescence.

AGR2 is a secreted protein, since AGR2 protein is detected in supernatants conditioned from HT-29 and LS174T, respectively, after supernatant concentration and immunoprecipitation using the before-mentioned anti-murine AGR2 antiserum, as shown in FIG. 20. Supernatants have been conditioned for 1 day and 3 days, respectively (IP Id conditioned supernatant, IP 3d conditioned supernatant). AGR2 protein is also detectable in the lysates cell pellet. In the present example, 20 μl of a Mon-1 specific rabbit antiserum were added to 10 ml of culture supernatants conditioned by 1×10⁷ cells for 24 and 72 hours, respectively. Following incubation, immunocomplexes containing Mon-1 protein were collected by adding immobilized protein A and resolved by SDS-PAGE. Immunoprecipitated Mon-1 protein was detected as described above.

### EXAMPLE 13

### Gene Therapy

A number of viruses, including retroviruses, adenoviruses, herpes viruses, and pox viruses, have been developed as live viral vectors for gene therapy. A nucleic acid that encodes for mutated AGR2 protein (SEQ ID NO:30) or wild type AGR2 protein (SEQ ID NO:4) is inserted into the genome of a parent virus to allow them to be expressed by that virus. This is accomplished by first constructing a DNA donor vector for in vivo recombination with a parent virus.

The DNA donor vector contains (i) a prokaryotic origin of replication, so that the vector may be amplified in a prokaryotic host; (ii) a gene encoding a marker which allows selection of prokaryotic host cells that contain the vector (e.g., a gene encoding antibiotic resistance); (iii) at least one gene encoding a desired protein located adjacent to a transcriptional promoter capable of directing the expression of the gene; and (iv) DNA sequences homologous to the region of the parent virus genome where the foreign gene(s) will be inserted, flanking the construct of element (iii).

The donor vector further contain additional genes which encodes one or more marker which will allow identification of recombinant viruses containing inserted foreign DNA. The marker genes to be used include genes that encode antibiotic or chemical resistance (e.g., see Spyropoulos et al., J. Virol., 62:1046 (1988); Falkner and Moss., J. Virol., 62:1849 (1988); Franke et al., Mol. Cell. Biol., 5:1918 (1985), as well as genes such as the E. coli lacZ gene, that permit identification of recombinant viral plaques by calorimetric assay (Panicali et al., Gene, 47:193-199 (1986)).

Homologous recombination between donor plasmid DNA and viral DNA in an infected cell are made using standard techniques. The recombination results in the formation of recombinant viruses that incorporate the nucleic acid encoding SEQ ID NO:29 for human mutated AGR2 or SEQ ID NO:5 for human wild type AGR2. Appropriate host cells for in vivo recombination are eukaryotic cells that can be infected by the virus and transfected by the plasmid vector such as chick embryo fibroblasts, HuTK143 (human) cells, and CV-1 and BSC-40 (both monkey kidney) cells. Infection of cells by the virus and transfection of these cells with plasmid vectors is accomplished by techniques standard in the art.

Following in vivo recombination, recombinant viral progeny are identified by co-integration of a gene encoding a marker or indicator gene with the foreign gene(s) of interest, which, in this case, is the β-galactosidase gene. The presence of the β-galactosidase gene is selected using the chromogenic substrate 5-bromo-4-chloro-3-indolyl-β-D-galactosidase (Panicali et al., Gene, 47:193 (1986)). Recombinant virus appears as blue plaques in the host cell. Expression of the polypeptide encoded by the inserted gene is further confirmed by in situ enzyme immunoassay performed on viral plaques and confirmed by Western blot analysis, radioimmunoprecipitation (RIPA), and enzyme immunoassay (EIA). Positive viruses are cultured and expanded and stored.

### EXAMPLE 14

### siRNA Generation and Use in Therapy

### Production of RNAs

Sense RNA (ssRNA) and antisense RNA (asRNA) of AGR2 are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min, then cooled and annealed at room temperature for 12 to 16 h. The RNAs were precipitated and resuspended in lysis buffer (below). To monitor annealing, RNAs were electrophoresed in a to 2% agarose gel in TBE buffer and stained with ethidium bromide (Sambrook et al., Molecular Cloning. Cold Spring Harbor Laboratory Press, Plainview, N.Y. (1989)).

### Lysate Preparation

Untreated rabbit reticulocyte lysate (Ambion) are assembled according to the manufacturer's directions. dsRNA was incubated in the lysate at 30° C. for 10 min prior to the addition of mRNAs. Then AGR2 mRNAs are added and the incubation continued for an additional 60 min. The molar ratio of double stranded RNA and mRNA is about 200:1. The AGR2 mRNA is radiolabeled (using known techniques) and its stability is monitored by gel electrophoresis.

In a parallel experiment made with the same conditions, the double stranded RNA is internally radiolabeled with α-³²P-ATP. Reactions are stopped by the addition of 2× proteinase K buffer and deproteinized as described previously Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluted. The efficacy of these 21-23 mers for suppressing AGR2 transcription may be assayed in vitro using the same rabbit reticulocyte assay described above using 50 nanomolar of double stranded 21-23 mer for each assay. The sequence of these 21-23mers is then determined using standard nucleic acid sequencing techniques.

### RNA Preparation

21 nt RNAs, based on the sequence determined above, were chemically synthesized using Expedite RNA phosphoramidites and thymidine phosphoramidite (Proligo, Germany). Synthetic oligonucleotides were deprotected and gel-purified (Elbashir, S. M., Lendeckel, W. & Tuschl, T., Genes & Dev. 15, 188-200 (2001)), followed by Sep-Pak C18 cartridge (Waters, Milford, Mass., USA) purification (Tuschl, T., et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

### Cell Culture

Cell cultures that regularly express AGR2, including, but not limited to FDC-P1, J774A.1 and WEHI-231 cells, are propagated using standard conditions. 24 hours before transfection, at approx. 80% confluency, the cells are trypsinized and diluted 1:5 with fresh medium without antibiotics (1−3×10⁵ cells/ml) and transferred to 24-well plates (500 μl/well). Transfection is performed using a commercially available lypofection kit and AGR2 expression is monitored using standard techniques with positive and negative control. Positive control is cells that naturally express AGR2 while negative control is cells that do not express AGR2. It is seen that base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of AGR2 siRNA sequence and the use of such siRNA for in vitro suppression. In vivo suppression may be performed using the same siRNA using well known in vivo transfection or gene therapy transfection techniques.

This invention has been described in detail including the preferred embodiments thereof. However, it will be appreciated that those skilled in the art, upon consideration of this disclosure, may make modifications and improvements thereon without departing from the spirit and scope of the invention as set forth in the claims. All references, patents, patent applications and Genbank references recited in this patent application are hereby incorporated by reference in their entirety.

### EXAMPLE 15

### Method for the Production of Transgenic Non-Human Animals Carrying a Transgene of Agr2, Produced by Gene Targeting Technology

Transgenic mice carrying a mammalian Agr2 transgene are generated by either using the embryonic stem cell method, or the pronucleus method, both of them well-known methods in the art; preferably using the method of Nehls and Wattler, as described in WO 01/5127. For transgenic methods see also US patents U.S. Pat. No. 6,436,701, U.S. Pat. No. 6,018,097, U.S. Pat. No. 5,942,435, U.S. Pat. No. 5,824,837, U.S. Pat. No. 5,731,489, and U.S. Pat. No. 5,523,226.

### EXAMPLE 16

### Agr2 Signal Peptide Prediction

The publicly available program “SignalP V1.1” was used to predict the probabilities of N-terminal signal peptides in murine and human Agr2 (Nielsen et al., 1997). The C-score (raw cleavage site score) of “SignalP V1.1” represents the output score from networks trained to recognize cleavage sites vs. other sequence positions. It was trained to be high at position +1 (immediately after the cleavage site) and low at all other positions. The S-score (signal peptide score) of “SignalP V1.1” represents the output score from networks trained to recognize signal peptide vs. non-signal-peptide positions. It was trained to be high at all positions before the cleavage site and low at 30 positions after the cleavage site and in the N-terminals of non-secretory proteins. The Y-score (combined cleavage site score) of “SignalP V1.1” represents the prediction of cleavage site location is optimized by observing where the C-score is high and the S-score changes from a high to a low value. The Y-score formalizes this by combining the height of the C-score with the slope of the S-score. Specifically, the Y-score is a geometric average between the C-score and a smoothed derivative of the S-score (i.e., the difference between the mean S-score over d positions before and d positions after the current position, where d varies with the chosen network ensemble). All three scores are averages of five networks trained on different partitions of the data.

For mouse Agr2 the program predicts with a high probability an N-terminal signal sequence encoded by the amino acids 1 to 20, and a cleavage site between amino acid 20 and 21 (see FIG. 15A).

For human AGR2 the program predicts with a high probability an N-terminal signal sequence encoded by the amino acids 1 to 20, and a cleavage site between amino acid 20 and 21 (see FIG. 15B).

### EXAMPLE 17

### Amino Acid Comparison Between Mouse and Human AGR2

The open reading frame of the mouse and human AGR2 cDNAs described herein encode deduced proteins of each 175 amino acids in size. Structural analysis of the sequence reveals a high probability for a translocation signal peptide which is removed after passing through the membrane. In both peptides, the most probable cleavage point is between amino acid 20 and 21 (LA-RD in human; LA-KD in mouse), creating a mature protein of 155 aa each. Signal peptide prediction was performed as described in Example 16 and as shown in FIGS. 15A and 15B, using the website of Center for Biological Sequence Analysis, BioCentrum-DTU, Technical University of Denmark, www.cbs.dtu.dk). The degree of amino acid identity between mouse and human Agr2 peptide is 91%, whereas the degree of similarity reaches 95%.

### EXAMPLE 18

### Characterization of Agr2 Proteins from Different Species—Amino Acid Conservation

1. In an inter-species comparison of mouse, rat, and human Agr2 peptide amino acids, the overall degree of identity is almost 91%, whereas the degree of similarity reaches 95%. The high degree of amino acid identity and similarity is indicative for highly conserved residues between the species (see FIG. 16 and Table 1), indicating functional significance of these conserved residues in the peptides compared in this Example. The amino acid that is exchanged in the MTZ phenotype, 137V, is identical between the species compared.

2. In an inter-species comparison of mouse, rat, human and Xenopus laevis Agr2 peptide amino acids, the overall degree of identity is 67%, whereas the degree of similarity reaches 82%. The high degree of amino acid identity and similarity is indicative for highly conserved residues between the species (see FIG. 17 and Table 2), indicating functional significance of these conserved residues in the peptides compared in this Example. Again, the amino acid that is exchanged in the MTZ phenotype, 137V, is identical between the species compared.

3. In an inter-species comparison of mouse, rat, human, Xenopus laevis, and C. elegans Agr2 peptide amino acids, the overall degree of identity is 32%, whereas the degree of similarity reaches 46%. The degree of amino acid identity and similarity is indicative for highly conserved residues between the species (see FIG. 18 and Table 3), indicating functional significance of these conserved residues in the peptides compared. The amino acid exchanged in the MTZ phenotype, 137V, is identical between the species compared in this Example, except for C. elegans. The C. elegans AGR2 protein is bearing a similar, i.e., nonpolar and hydrophobic, amino acid at the corresponding residue position 137 (L instead of V).

Evolutionary pressure has conserved these residues at their particular locations in the molecule. It is predicted that any non-conservative aa substitution will modify the peptide's normal biological function in a manner analogous to that observed in the present invention. Hence, identification of such an abnormal Agr2 peptide sequence in a biological sample, or of the a cDNA encoding such an abnormal Agr2 peptide, will be indicative of an increased probability of developing the phenotype of the present invention.

### EXAMPLE 19

### Xenopus Laevis Cement Gland Differentiation Assay

A functional analysis of mouse Agr2 protein and orthologue AGR2 peptides can be performed in an assay described by Aberger et al. ((Aberger et al., 1998)). The authors demonstrated that overexpression of XAG-2, a secreted protein which acts specifically at cement glands induces both, ectopic cement gland differentiation and expression of anterior neural marker genes in Xenopus embryos. XAG-2 is a secreted protein homologue to AGR2.

The assay can be used as a test for particular genes function in the specification of the cement gland during embryonic development. The cement gland is a mucin secreting organ in Xenopus laevis embryos, being functionally similar to goblet cells.

A PCR fragment carrying a full-length Agr2 cDNA sequence, is subcloned into a plasmid vector, i.e. pCR 2.1-TOPO (K4500-01, Invitrogen, Carlsbad, Calif., USA), according to the manufacturer's instructions. The plasmid DNA, bearing the correct Agr2 insert is subject to site-directed mutagenesis, using a QuickChange Site-Directed Mutagenesis Kit (200518, Stratagene, La Jolla, Calif., USA), as described in Example 5.

Altering a particular codon sequence (which encodes a particular amino acid) by substitution of one, or two, or three base pairs of the codon, will give rise to AGR2 proteins bearing non-conservative amino acid exchanges at the residue positions indicated in Tables 1, 2, and 3, respectively.

Capped mRNA is synthesized with an SP6 mMessage mMachine Kit (Ambion). A small sample of mRNA is in vitro translated with a reticulocyte lysate system (Promega) to analyze the quality of RNAs; or with a different method as described, for instance, in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993. Purified mRNA is injected into early cleavage stage embryos of Xenopus laevis, as described in Aberger et al., 1998.

Depending on the point mutations and on the subsequent non-conserved amino acid substitutions introduced (at the residue positions listed in the Tables 1, 2, and 3, respectively), AGR2 function is analyzed in respect to specification of mucin secreting cement glands. Morphological and histological examinations are performed to analyze for cement gland enlargement or additional ectopic cement glands, as described in Aberger et al.

### EXAMPLE 20

### Agr2 Function in Cell Proliferation—DNA Labeling in a Growth Factor Assay

To measure AGR2 activity in cell proliferation, a DNA labeling assay can be used. For mammalian AGR2, colon cancer cell lines like LS174T or HT29, can be used. LS174T cells exhibit a goblet cell-like phenotype producing significant amounts of secretory mucin, as described by Iwakira and Podolsky (Am. J. Physiol Gastrointest Liver Physiol 280: G1114-G1123, 2001). HT29 cells can differentiate into cells with phenotypical characteristics of enterocytes and mucin-secreting goblet cells. Any other cells, which are responsive to AGR2 can be used.

AGR2 expression vectors, bearing wt and mutated cDNA sequences of an mammalian Agr2 gene, and additional control vectors are constructed as described in Example 10. A preferred method is the cDNA subcloning into expression vectors of the Gateway cloning and expression system (Invitrogen, California, USA), according to the manufacturer's instructions.

There are several protocols to perform cell proliferation assays that are well known in the art. Typically, the incorporation of a nucleoside analog into newly synthesized DNA is employed to measure proliferation (active cell growth) in a population of cells. For example, Bromodeoxyuridine (BrdU) can be employed as a DNA labeling reagent and Anti-BrdU mouse monoclonal antibody can be employed as a detection reagent. This antibody binds only to cells containing DNA which has incorporated BrdU. A number of detection methods can be used in conjunction with this assay including immunofluorescence, immunohistochemical, ELISA and calorimetric methods. Kits that include BrdU and anti-BrdU mouse monoclonal antibody are commercially available from F. Hoffmann-La Roche Ltd (Basel, Switzerland). The assay is performed as indicated in the manufacturer's protocol.

### EXAMPLE 21

### Agr2 Function in Goblet Cell Differentiation—Analysis of Goblet Cell Specific Markers in a Quantitative PCR Assay

To measure AGR2 activity in goblet cell differentiation, e.g., in either early or terminal goblet cell differentiation, a cell culture based assay can be used. For mammalian AGR2, colon cancer cell lines like LS174T or HT29, can be used. LS174T cells exhibit a goblet cell-like phenotype producing significant amounts of secretory mucin, as described by Iwakira and Podolsky (Am. J. Physiol Gastrointest Liver Physiol 280: G1114-G1123, 2001). HT29 cells can differentiate into cells with phenotypical characteristics of enterocytes and mucin-secreting goblet cells.

AGR2 expression vectors, bearing wt and mutated cDNA sequences of an mammalian Agr2 gene, and additional control vectors are constructed as described in Example 10. A preferred method is the cDNA subcloning into expression vectors of the Gateway cloning and expression system (Invitrogen, California, USA), according to the manufacturer's instructions.

Cells are transfected with expression vectors as described above. Transfection of culture cells with expression vectors is well known in the art and described, for instance, in Sambrook et al. (eds.), MOLECULAR CLONING: A LABORATORY MANUAL (2^{nd} Ed.), Cold Spring Harbor Laboratory Press, Cold Spring Harbor, N.Y., 1989; and Ausubel et al. (eds.), CURRENT PROTOCOLS IN MOLECULAR BIOLOGY, John Wiley & Sons, New York, N.Y., 1993.

A major mucin subtype secreted by intestinal goblet cells is mucin2 (muc2). Mucin2 serves, like mucin subtype TFF3, as a marker for terminal differentiation. Human muc2 primers are designed to PCR amplify an about 200 bp DNA fragment at cDNA, which is freshly synthesized at mRNA of transfected and non-transfected controle cells. The quantitative PCR analysis (Light cycler; Roche, Basel, Switzerland) is performed, according to the manufacturer's instruction.

AGR2 function in goblet cell differentiation is analyzed by quantitative determination of human muc2 PCR products. The amount of specific PCR product is depending on the particular type of AGR2 expression vector (wild type cDNA, mutated cDNA, position of mutation) used for transfection. The analysis is not limited to muc2.

### EXAMPLE 22

### AGR2 Mutations Resulting in Abnormal AGR2 Protein Expression Levels

It is predicted that any mutation in the AGR2 gene resulting in abnormal AGR2 peptide expression levels in an individual will interfere with the peptide's normal biological function, including in a manner analogous to that observed in the present invention. Mutations leading to abnormal AGR2 peptide expression levels might affect any aspect of gene expression, e.g. DNA transcription, mRNA transport and processing, mRNA translation or AGR2 peptide half-life itself.

For instance, identification of an abnormal AGR2 peptide level in a biological sample will be indicative of an increased probability of developing the phenotype of the present invention. Methods for quantifying the peptide expression levels in a biological sample are well known in the art. AGR2 peptide levels could be analysed by obtaining a biopsy from an individual and quantifying the amount of AGR2 peptide by the use of an antibody or any other probe specifically recognizing the AGR2 peptide, e.g. using an ELISA or a Western Blot.

Alternatively, identification of an abnormal AGR2 mRNA level in a biological sample will be indicative of an increased probability of developing the phenotype of the present invention. Methods for quantifying the mRNA expression levels in a biological sample are well known in the art. AGR2 mRNA levels could be analysed by obtaining a biopsy from an individual and quantifying the amount of AGR2 mRNA by the use of quantitative RT-PCR or any other method relying on probes specifically recognizing the AGR2 mRNA.

Alternatively, identification of an abnormal AGR2 mRNA transport and processing in a biological sample will be indicative of an increased probability of developing the phenotype of the present invention. AGR2 mRNA processing could be analysed by obtaining a biopsy from an individual and quantifying the processing of AGR2 mRNA by the use of Northern blotting or qualitative RT-PCR or any other method relying on probes specifically recognizing the AGR2 mRNA processing.

Moreover, any given mutation in the AGR2 gene could be tested for its effect on AGR2 expression by using an appropriate artificial expression system.

For instance, a cDNA encoding any given mutated AGR2 peptide could be isolated and expressed in any suitable expression system. The amount of expressed AGR2 peptide or mRNA or the AGR2 mRNA transport and processing could be analysed by using methods analogous to those mentioned above.

Alternatively, regulatory sequences of the AGR2 gene could be isolated and analysed in any suitable expression system. Expression levels of an appropriate reporter gene would be indicative for the efficiency of the AGR2 regulatory sequences to direct gene expression.

Once mutations in the AGR2 gene resulting in abnormal AGR2 peptide expression levels in an individual or in a suitable expression system are identified, this knowledge might be used to screen any suitable biological sample for presence of such a mutation by means well known in the art, including sequencing of the individual's AGR2 cDNA or genomic DNA. Individuals carrying any of the previously characterized mutations will bare an increased risk of developing the phenotype of the present invention.

### EXAMPLE 23

### Statistical Analysis of Populations to identify Correlations Between AGR2 Haplotype and Disease Risk

In order to identify mutants of the human AGR2 gene, which are indicative of an increased probability of developing the phenotype described by the present invention, the AGR2 haplotypes are determined from defined collectives of patients displaying a disease phenotype reminiscent to that described in the present invention in comparison to a suitable healthy control population. AGR2 alleles, which are significantly over-represented in the affected population versus the control population are correlated with the disease risk, see in Griffiths, Anthony J. F.; Gelbart, William M.; Miller, Jeffrey H.; Lewontin, Richard C. Modern Genetic Analysis. New York: W H Freeman & Co; c1999.

Therefore, individuals carrying any of these over-represented AGR2 alleles will bare an increased risk of developing the phenotype of the present invention.

### EXAMPLE 24

### Detection of Transcriptionally Deregulated Genes Expressed in the Colon

A series of genes selected for their putative biological relevance to goblet cell function were analysed for altered RNA expression levels in the colon of newborn MTZ mice, in comparison to expression levels in colon of wild type mice. Significantly reduced expression levels were found for Mucin2 (Muc2) and Trefoil factor 3 (TFF3), as shown in FIG. 19. Both genes encode the major protein components of mucin and both proteins, Muc-2 and TFF3, serve as marker for late goblet cell differentiation. Reduced transcriptional activity of these differentiation marker genes is indicative of an incomplete maturation process of the goblet cells. Transcriptional deregulation was determined by quantitative PCR-Light Cycler technology (Roche Diagnostics GmbH, Mannheim, Germany), according to the manufacturer's instructions.

TABLE 1
Conserved amino acid residues in mouse, rat, and human
a) identical residues

| | | | | | | | | | | | | | | |
|---|---|---|---|---|---|---|---|---|---|---|---|---|---|---|
| M1 | E2 | K3 | V6 | S7 | A8 | L10 | L11 | L12 | V13 | A14 | S16 | T18 | L19 | A20 |
| D22 | T23 | T24 | V25 | K26 | G28 | K30 | K31 | D32 | K34 | D35 | S36 | R37 | P38 | K39 |
| L40 | P41 | Q42 | T43 | L44 | S45 | R46 | G47 | W48 | G49 | D50 | Q51 | L52 | I53 | W54 |
| T55 | Q56 | T57 | Y58 | E59 | E60 | A61 | L62 | Y63 | S65 | K66 | T67 | S68 | N69 | P71 |
| L72 | M73 | I75 | H76 | H77 | L78 | D79 | E80 | C81 | P82 | H83 | S84 | Q85 | A86 | L87 |
| K88 | K89 | V90 | F91 | A92 | E93 | K95 | E96 | I97 | Q98 | K99 | L100 | A101 | E102 | Q103 |
| F104 | V105 | L106 | L107 | N108 | L109 | Y111 | E112 | T113 | T114 | D115 | K116 | H117 | L118 | S119 |
| P120 | D121 | G122 | Q123 | Y124 | V125 | P126 | R127 | I128 | F130 | V131 | D132 | P133 | S134 | L135 |
| T136 | V137 | R138 | A139 | D140 | I141 | T142 | G143 | R144 | Y145 | S146 | N147 | R148 | L149 | Y150 |
| A151 | Y152 | E153 | P154 | D156 | T157 | A158 | L159 | L160 | D162 | N163 | M164 | K165 | K166 | A167 |
| L168 | K169 | L170 | L171K | T173 | E174 | L175 | | | | | | | | |

b) similar residues

| | | | | | | |
|---|---|---|---|---|---|---|
| I or L15 | K or R21 | A or S29 | K or R64 | R or K70 | V or I73 | V or I110 |
| V or M129 | S or A155 | | | | | |

Explanation of amino acid single letter code:
A = Ala
H = His
T = Thr
R = Arg
I = Ile
W = Trp
N = Asn
L = Leu
Y = Tyr
D = Asp
K = Lys
V = Val
C = Cys
M = Met
E = Glu
F = Phe
Q = Gln
P = Pro
G = Gly
S = Ser

TABLE 2
Conserved amino acid residues in mouse, rat, human,
and Xenopus.
a) identical residues in respect to mouse, rat, and human
amino acid positions.

| | | | | | | | | | | | | | | |
|---|---|---|---|---|---|---|---|---|---|---|---|---|---|---|
| M1 | E2 | S7 | L11 | L12 | V13 | A14 | S16 | T18 | L19 | A20 | P41 | Q42 | T43 | L44 |
| S45 | R46 | G47 | W48 | G49 | D50 | L52 | W54 | Q56 | T57 | Y58 | E59 | E60 | L62 | K66 |
| N69 | P71 | L72 | I75 | H77 | C81 | P82 | H83 | S84 | Q85 | A86 | L87 | K88 | K89 | F91 |
| A92 | E93 | I97 | Q98 | K99 | L100 | A101 | E102 | F104 | L106 | L107 | N108 | L109 | Y111 | T114 |
| D115 | K116 | L118 | D121 | G122 | Q123 | Y124 | V125 | P126 | F130 | V131 | D132 | P133 | S134 | L135 |
| V137 | R138 | A139 | D140 | G143 | Y145 | S146 | N147 | Y150 | Y152 | E153 | P154 | D156 | L160 | N163 |
| M164 | K165 | K166 | A167 | L168 | L170 | L171K | T173 | E174 | L175 | | | | | |

b) similar residues in respect to mouse, rat, and human
amino acid positions.

| | | | | | | | |
|---|---|---|---|---|---|---|---|
| I or L15 | K or R20 | D or E21 | A or S29 | K or R39 | Q or N51 | A or G61 | Y orF63 |
| K or R64 | S or A65 | T or S67 | R or K70 | M or L73 | V or I or L74 | D or N79 | E orD80 |
| Q or E103 | V or I105 | L or I109 | V or I110 | P or K127 | I or V128 | V or M129 | I or L141 |
| R or K144 | R or H148 | D or E161 | | | | | |

Explanation of amino acid single letter code:
A = Ala
H = His
T = Thr
R = Arg
I = Ile
W = Trp
N = Asn
L = Leu
Y = Tyr
D = Asp
K = Lys
V = Val
C = Cys
M = Met
E = Glu
F = Phe
Q = Gln
P = Pro
G = Gly
S = Ser

TABLE 3
Conserved amino acid residues in mouse, rat, human,
Xenopus, and C. elegans.
a) identical residues in respect to mouse, rat, and human
amino acid positions.

| | | | | | | | | | | | | | | |
|---|---|---|---|---|---|---|---|---|---|---|---|---|---|---|
| S7 | L12 | L44 | R46 | G47 | G49 | D50 | W54 | E59 | P71 | H77 | C81 | A86 | L87 | K88 |
| K89 | F91 | K99 | L100 | E102 | F104 | N108 | D121 | G122 | Y124 | F130 | D132 | Y150 | Y152 | D132 |
| M164 | K165 | L168 | | | | | | | | | | | | |

b) similar residues in respect to mouse, rat, and human
amino acid positions.

| | | | | | | | |
|---|---|---|---|---|---|---|---|
| I or L15 | K or R20 | D or E21 | A or S29 | K or R39 | Q or N51 | A or G61 | Y or F63 |
| K or R64 | S or A65 | T or S67 | R or K70 | M or L73 | V or I or L74 | D or N79 | E or D80 |
| Q or E103 | V or I105 | L or I109 | V or I110 | P or K127 | I or V128 | V or M129 | I or L141 |
| R or K144 | R or H148 | D or E161 | V or L13 | S or A16 | Q or N42 | S or A45 | W or F48 |
| L or I52 | Y or W58 | E or D60 | L or I62 | N or D69 | L or I72 | I or L75 | E or Q93 |
| A or S101 | L or M106 | L or V107 | D or E115 | V or I125 | V or L131 | V or L137 | S or A 146 |
| L or I160 | E or D174 | | | | | | |

Explanation of amino acid single letter code:
A = Ala
H = His
T = Thr
R = Arg
I = Ile
W = Trp
N = Asn
L = Leu
Y = Tyr
D = Asp
K = Lys
V = Val
C = Cys
M = Met
E = Glu
F = Phe
Q = Gln
P = Pro
G = Gly
S = Ser

### REFERENCES

1. Aberger, F., Weidinger, G., Grunz, H., and Richter, K. (1998). Anterior specification of embryonic ectoderm: the role of the Xenopus cement gland-specific gene XAG-2. Mech. Dev. 72, 115-130.
2. Ahlstedt, S, and Enander, I. (1987). Immune regulation of goblet cell development. Int. Arch. Allergy Appl. Immunol. 82, 357-360.
3. Bhat, S. P. (2001). The ocular lens epithelium. Biosci. Rep. 21, 537-563.
4. Brennan, M., Davison, P. F., and Paulus, H. (1985). Preparation of bispecific antibodies by chemical recombination of monoclonal immunoglobulin G1 fragments. Science 229, 81-83.
5. Brittan, M. and Wright, N. A. (2002). Gastrointestinal stem cells. J. Pathol. 197, 492-509.
6. Caron P. C., Laird, W., Co, M. S., Avdalovic, N. M., Queen, C., and Scheinberg, D. A. (1992). Engineered humanized dimeric forms of IgG are more effective antibodies. J. Exp. Med. 176, 1191-1195.
7. Corfield, A. P., Carroll, D., Myerscough, N., and Probert, C. S. (2001). Mucins in the gastrointestinal tract in health and disease. Front Biosci. 6:D1321-57., D1321-D1357.
8. Cote, R. J., Morrissey, D. M., Houghton, A. N., Beattie, E. J., Jr., Oettgen, H. F., and Old, L. J. (1983). Generation of human monoclonal antibodies reactive with cellular antigens. Proc. Natl. Acad. Sci. U. S. A 80, 2026-2030.
9. Daniels, J. T., Dart, J. K., Tuft, S. J., and Khaw, P. T. (2001). Corneal stem cells in review. Wound. Repair Regen. 9, 483-494.
10. Dekker, J., Rossen, J. W., Buller, H. A., and Einerhand, A. W. (2002). The MUC family: an obituary. Trends Biochem. Sci. 27, 126-131.
11. Deplancke, B. and Gaskins, H. R. (2001). Microbial modulation of innate defense: goblet cells and the intestinal mucus layer. Am. J. Clin. Nutr. 73, 1131S-1141S.
12. Einerhand, A. W., Renes, I. B., Makkink, M. K., van der, S. M., Buller, H. A., and Dekker, J. (2002). Role of mucins in inflammatory bowel disease: important lessons from experimental models. Eur. J. Gastroenterol. Hepatol. 14, 757-765.
13. Elbashir, S. M., Lendeckel, W., and Tuschl, T. (2001a). RNA interference is mediated by 21- and 22-nucleotide RNAs. Genes Dev. 15, 188-200.
14. Elbashir, S. M., Martinez, J., Patkaniowska, A., Lendeckel, W., and Tuschl, T. (2001b). Functional anatomy of siRNAs for mediating efficient RNAi in Drosophila melanogaster embryo lysate. EMBO J. 20, 6877-6888.
15. Emura, M. (2002). Stem cells of the respiratory tract. Paediatr. Respir. Rev. 3, 36-40.
16. Fahy, J. V. (2001). Remodeling of the airway epithelium in asthma. Am. J. Respir. Crit. Care Med. 164, S46-S51.
17. Farrell, C. L., Rex, K. L., Chen, J. N., Bready, J. V., DiPalma, C. R., Kaufman, S. A., Rattan, A., Scully, S., and Lacey, D. L. (2002). The effects of keratinocyte growth factor in preclinical models of mucositis. Cell Prolif. 35 Suppl 1:78-85., 78-85.
18. Fishwild, D. M., O'Donnell, S. L., Bengoechea, T., Hudson, D. V., Harding, F., Bernhard, S. L., Jones, D., Kay, R. M., Higgins, K. M., Schramm, S. R., and Lonberg, N. (1996a). High-avidity human IgG kappa monoclonal antibodies from a novel strain of minilocus transgenic mice. Nat. Biotechnol. 14, 845-851.
19. Fishwild, D. M., O'Donnell, S. L., Bengoechea, T., Hudson, D. V., Harding, F., Bernhard, S. L., Jones, D., Kay, R. M., Higgins, K. M., Schramm, S. R., and Lonberg, N. (1996b). High-avidity human IgG kappa monoclonal antibodies from a novel strain of minilocus transgenic mice. Nat. Biotechnol. 14, 845-851.
20. Forstner, J. F. (1978). Intestinal mucins in health and disease. Digestion 17, 234-263.
21. Foster, C. S., Dodson, A., Karavana, V., Smith, P. H., and Ke, Y. (2002). Prostatic stem cells. J. Pathol. 197, 551-565.
22. Gruber, M., Schodin, B. A., Wilson, E. R., and Kranz, D. M. (1994). Efficient tumor cell lysis mediated by a bispecific single chain antibody expressed in Escherichia coli. J. Immunol. 152, 5368-5374.
23. Harborth, J., Elbashir, S. M., Bechert, K., Tuschl, T., and Weber, K. (2001). Identification of essential genes in cultured mammalian cells using small interfering RNAs. J. Cell Sci. 114, 4557-4565.
24. Higgins, D. G. and Sharp, P. M. (1988). CLUSTAL: a package for performing multiple sequence alignment on a microcomputer. Gene 73, 237-244.
25. Holliger P., Prospero, T., and Winter, G. (1993). “Diabodies”: small bivalent and bispecific antibody fragments. Proc. Natl. Acad. Sci. U. S. A 90, 6444-6448.
26. Hoogenboom, H. R. and Winter, G. (1992). By-passing immunisation. Human antibodies from synthetic repertoires of germline VH gene segments rearranged in vitro. J. Mol. Biol. 227, 381-388.
27. Hopp, T. P. and Woods, KR (1981). Prediction of protein antigenic determinants from amino acid sequences. Proc. Natl. Acad. Sci. U. S. A 78, 3824-3828.
28. Huse, W. D., Sastry, L., Iverson, S. A., Kang, A. S., Alting-Mees, M., Burton, D. R., Benkovic, S. J., and Lerner, R. A. (1989). Generation of a large combinatorial library of the immunoglobulin repertoire in phage lambda. Science 246, 1275-1281.
29. Jackson, A. D. (2001). Airway goblet-cell mucus secretion. Trends Pharmacol. Sci. 22, 39-45.
30. Jass, J. R. and Walsh, M. D. (2001). Altered mucin expression in the gastrointestinal tract: a review. J. Cell Mol. Med. 5, 327-351.
31. Jones, P. T., Dear, P. H., Foote, J., Neuberger, M. S., and Winter, G. (1986). Replacing the complementarity-determining regions in a human antibody with those from a mouse. Nature 321, 522-525.
32. Katz, J. P., Perreault, N., Goldstein, B. G., Lee, C. S., Labosky, P. A., Yang, V. W., and Kaestner, K. H. (2002). The zinc-finger transcription factor Klf4 is required for terminal differentiation of goblet cells in the colon. Development 129, 2619-2628.
33. Kohler, G. and Milstein, C. (1975). Continuous cultures of fused cells secreting antibody of predefined specificity. Nature 256, 495-497.
34. Komiya, T., Tanigawa, Y., and Hirohashi, S. (1999). Cloning of the gene gob-4, which is expressed in intestinal goblet cells in mice. Biochim. Biophys. Acta 1444, 434-438.
35. Kostelny, S. A., Cole, M. S., and Tso, J. Y. (1992). Formation of a bispecific antibody by the use of leucine zippers. J. Immunol. 148, 1547-1553.
36. Kozbor, D., Tripputi P., Roder, J. C., and Croce, C. M. (1984). A human hybrid myeloma for production of human monoclonal antibodies. J. Immunol. 133, 3001-3005.
37. Kyte, J. and Doolittle, R. F. (1982a). A simple method for displaying the hydropathic character of a protein. J. Mol. Biol. 157, 105-132.
38. Kyte, J. and Doolittle, R. F. (1982b). A simple method for displaying the hydropathic character of a protein. J. Mol. Biol. 157, 105-132.
39. Laboisse, C., Jarry, A., Branka, J. E., Merlin, D., Bou-Hanna, C., and Vallette, G. (1996). Recent aspects of the regulation of intestinal mucus secretion. Proc. Nutr. Soc. 55, 259-264.
40. Lonberg, N. and Huszar, D. (1995b). Human antibodies from transgenic mice. Int. Rev. Immunol. 13, 65-93.
41. Lonberg, N. and Huszar, D. (1995a). Human antibodies from transgenic mice. Int. Rev. Immunol. 13, 65-93.
42. Lonberg, N., Taylor, L. D., Harding, F. A., Trounstine, M., Higgins, K. M., Schramm, S. R., Kuo, C. C., Mashayekh, R., Wymore, K., McCabe, J. G., and (1994a). Antigen-specific human antibodies from mice comprising four distinct genetic modifications. Nature 368, 856-859.
43. Lonberg, N., Taylor, L. D., Harding, F. A., Trounstine, M., Higgins, K. M., Schramm, S. R., Kuo, C. C., Mashayekh R., Wymore, K., McCabe, J. G., and (1994b). Antigen-specific human antibodies from mice comprising four distinct genetic modifications. Nature 368, 856-859.
44. Maestrelli, P., Saetta, M., Mapp, C. E., and Fabbri, L. M. (2001). Remodeling in response to infection and injury. Airway inflammation and hypersecretion of mucus in smoking subjects with chronic obstructive pulmonary disease. Am. J. Respir. Crit. Care Med. 164, S76-S80.
45. Marks, J. D., Griffiths, A. D., Malmqvist, M., Clackson, T. P., Bye, J. M., and Winter, G. (1992). By-passing immunization: building high affinity human antibodies by chain shuffling. Biotechnology (N. Y.) 10, 779-783.
46. Marks, J. D., Hoogenboom, H. R., Bonnert, T. P., McCafferty, J., Griffiths, A. D., and Winter, G. (1991a). By-passing immunization. Human antibodies from V-gene libraries displayed on phage. J. Mol. Biol 222, 581-597.
47. Marks, J. D., Hoogenboom, H. R., Bonnert, T. P., McCafferty, J., Griffiths, A. D., and Winter, G. (1991b). By-passing immunization. Human antibodies from V-gene libraries displayed on phage. J. Mol. Biol 222, 581-597.
48. Melton, L. (2002). Does mucus hypersecretion matter in airway disease Lancet 359, 1924.
49. Milstein, C. and Cuello, A. C. (1983). Hybrid hybridomas and their use in immunohistochemistry. Nature 305, 537-540.
50. Morrison, S. L. (1994b). Immunology. Success in specification. Nature 368, 812-813.
51. Morrison, S. L. (1994a). Immunology. Success in specification. Nature 368, 812-813.
52. Munson, P. J. and Rodbard, D. (1980). Ligand: a versatile computerized approach for characterization of ligand-binding systems. Anal. Biochem. 107, 220-239.
53. Nadel, J. A. (2001). Role of epidermal growth factor receptor activation in regulating mucin synthesis. Respir. Res. 2, 85-89.
54. Neuberger, M. (1996a). Generating high-avidity human Mabs in mice. Nat. Biotechnol. 14, 826.
55. Neuberger, M. (1996b). Generating high-avidity human Mabs in mice. Nat. Biotechnol. 14, 826.
56. Nielsen, H., Engelbrecht, J., Brunak, S., and von Heijne, G. (1997). Identification of prokaryotic and eukaryotic signal peptides and prediction of their cleavage sites. Protein Eng 10, 1-6.
57. Otto, W. R. (2002). Lung epithelial stem cells. J. Pathol. 197, 527-535.
58. Podolsky, D. K. (2000). Mechanisms of regulatory peptide action in the gastrointestinal tract: trefoil peptides. J. Gastroenterol. 35 Suppl 12:69-74., 69-74.
59. Puchelle, E., Bajolet, O., and Abely, M. (2002). Airway mucus in cystic fibrosis. Paediatr. Respir. Rev. 3, 115.
60. Riechmann, L., Clark, M., Waldmann, H., and Winter, G. (1988b). Reshaping human antibodies for therapy. Nature 332, 323-327.
61. Riechmann, L., Clark, M., Waldmann, H., and Winter, G. (1988a). Reshaping human antibodies for therapy. Nature 332, 323-327.
62. Schnoelzer, M., Alewood, P., Jones, A., Alewood, D., Kent, S. B. (1992). I situ neutralization in Boc-chemistry solid phase peptide synthesis. Rapid, high yield assembly of difficult sequences. Int. J. Pept Protein Res. 40(3-4): 180-193.
63. Schreiber, J., Bohnsteen, B., and Rosahl, W. (2002). Influence of mucolytic therapy on respiratory mechanics in patients with chronic obstructive pulmonary disease. Eur. J. Med. Res. 7, 98-102.
64. Shalaby, M. R., Shepard, H. M., Presta, L., Rodrigues, M. L., Beverley, P. C., Feldmann, M., and Carter, P. (1992). Development of humanized bispecific antibodies reactive with cytotoxic lymphocytes and tumor cells overexpressing the HER2 protooncogene. J. Exp. Med. 175, 217-225.
65. Shopes, B. (1992b). A genetically engineered human IgG mutant with enhanced cytolytic activity. J. Immunol. 148, 2918-2922.
66. Shopes, B. (1992a). A genetically engineered human IgG mutant with enhanced cytolytic activity. J. Immunol. 148, 2918-2922.
67. Skerra, A. (2001). ‘Anticalins’: a new class of engineered ligand-binding proteins with antibody-like properties. J. Biotechnol. 74(4):257-275
68. Slomiany, B. L. and Slomiany, A. (2002). Disruption in gastric mucin synthesis by Helicobacter pylori lipopolysaccharide involves ERK and p38 mitogen-activated protein kinase participation. Biochem. Biophys. Res. Commun. 294, 220-224.
69. Specian, R. D. and Oliver, M. G. (1991). Functional biology of intestinal goblet cells. Am. J. Physiol 260, C183-C193.
70. Stappenbeck, T. S. and Gordon, J. I. (2000). Rac1 mutations produce aberrant epithelial differentiation in the developing and adult mouse small intestine. Development 127, 2629-2642.
71. Stevenson, G. T., Pindar, A., and Slade, C. J. (1989). A chimeric antibody with dual Fc regions (bisFabFc) prepared by manipulations at the IgG hinge. Anticancer Drug Des 3, 219-230.
72. Suresh, M. R., Cuello A. C., and Milstein, C. (1986). Advantages of bispecific hybridomas in one-step immunocytochemistry and immunoassays. Proc. Natl. Acad. Sci. U. S. A 83, 7989-7993.
73. Thompson, D. A. and Weigel, R. J. (1998). hAG-2, the human homologue of the Xenopus laevis cement gland gene XAG-2, is coexpressed with estrogen receptor in breast cancer cell lines. Biochem. Biophys. Res. Commun. 251, 111-116.
74. Traunecker, A., Lanzavecchia, A., and Karjalainen, K. (1991). Bispecific single chain molecules (Janusins) target cytotoxic lymphocytes on HIV infected cells. EMBO J. 10, 3655-3659.
75. Tuschl, T., Zamore, P. D., Lehmann, R, Bartel, D. P., and Sharp, P. A. (1999). Targeted mRNA degradation by double-stranded RNA in vitro. Genes Dev. 13, 3191-3197.
76. Tutt, A., Stevenson, G. T., and Glennie, M. J. (1991). Trispecific F(ab′)3 derivatives that use cooperative signaling via the TCR/CD3 complex and CD2 to activate and redirect resting cytotoxic T cells. J. Immunol. 147, 60-69.
77. van Den Brink, G. R., de Santa, B. P., and Roberts, D. J. (2001). Development. Epithelial cell differentiation—a Mather of choice. Science 294, 2115-2116.
78. Velcich, A., Yang, W., Heyer, J., Fragale, A., Nicholas, C., Viani, S., Kucherlapati, R., Lipkin, M., Yang, K., and Augenlicht, L. (2002). Colorectal cancer in mice genetically deficient in the mucin Muc2. Science 295, 1726-1729.
79. Verdugo, P. (1990). Goblet cells secretion and mucogenesis. Annu. Rev. Physiol 52:157-76., 157-176.
80. Verdugo, P. (1991). Mucin exocytosis. Am. Rev. Respir. Dis. 144, S33-S37.
81. Verhoeyen, M., Milstein, C., and Winter, G. (1988a). Reshaping human antibodies: grafting an antilysozyme activity. Science 239, 1534-1536.
82. Verhoeyen, M., Milstein, C., and Winter, G. (1988b). Reshaping human antibodies: grafting an antilysozyme activity. Science 239, 1534-1536.
83. Vitetta, E. S., Krolick, K. A., Miyama-Inaba, M., Cushley, W., and Uhr, J. W. (1983). Immunotoxins: a new approach to cancer therapy. Science 219, 644-650.
84. Voynow, J. (2002). What does mucin have to do with lung disease Paediatr. Respir. Rev. 3, 98.
85. Watanabe, H. (2002). Significance of mucin on the ocular surface. Cornea 21, S17-S22.
86. Weiss, G. A., Lowman, H. B. (2001). Anticalins versus antibodies: made-to-order binding proteins for small molecules. Chem. Biol. 7(8), R177-184
87. Wolff, E. A., Schreiber, G. J., Cosand, W. L., and Raff, H. V. (1993). Monoclonal antibody homodimers: enhanced antitumor activity in nude mice. Cancer Res. 53, 2560-2565.
88. Yang, Q., Bermingham, N. A., Finegold, M. J., and Zoghbi, H. Y. (2001). Requirement of Math1 for secretory cell lineage commitment in the mouse intestine. Science 294, 2155-2158.

### SUMMARY OF SEQUENCES

### SEQ ID NO:1: Agr2 mouse nuc-seq Mutant C3H

### SEQ ID NO:2: Agr2 mouse prot-seq Mutant

### SEQ ID NO:3: Agr2 mouse prot-seq WT

### SEQ ID NO:4: AGR2 human prot-seq WT

### SEQ ID NO:5: AGR2 human nuc-seq WT

### SEQ ID NO:6: Agr2 mouse nuc-seq WT

### SEQ ID NO:7: mAgr2-7 primer

### SEQ ID NO:8: mAgr2-2 primer

### SEQ ID NO:9: hAgr-1 primer

### SEQ ID NO:10: hAgr-4 primer

### SEQ ID NO:11: Idb2-SNP-marker

### SEQ ID NO:12: primer1 Idb2-SNP-marker

### SEQ ID NO:13: primer2 Idb2-SNP-marker

### SEQ ID NO:14: D12Mit64 MIT-marker

### SEQ ID NO:15: primer1 D12Mit64 MIT-marker

### SEQ ID NO:16: primer2 D12Mit64 MIT-marker

### SEQ ID NO:17-28: agr2 primers 1-12

### SEQ ID NO:29: AGR2 human nuc-seq Mutant

### SEQ ID NO:30: AGR2 human prot-seq Mutant

### SEQ ID NO:31 hAgr2-3 primer

### SEQ ID NO:32: hAgr2-4 primer

### SEQ ID NO:33: PCR product of hAgr2-3 and hAgr24

### SEQ ID NO:3 listed below (i.e., the wild type mouse Agr2 protein sequence) corresponds to the sequence to be found in Genbank under accession number NP_{—}035913.

### SEQ ID NO:4 listed below (i.e., the wild type human AGR2 protein sequence) corresponds to the sequence to be found in Genbank under accession number NP_{—}006399.

SEQ ID NO:1 nucleic acid sequence (cDNA) of mutant Agr2
(mus musculus; C3H)
GGCAACCCTTGCGGCTCACACAAAGCAGGAGGGTGGGAAGCCCAGATTTGCCATGGAGAAATTTTC
AGTGTCTGCAATCCTGCTTCTTGTGGCCATTTCTGGTACCTTGGCCAAAGACACCACAGTCAAATC
TGGAGCCAAAAAGGACCCAAAGGACTCTCGGCCCAAACTACCTCAGACACTCTCCAGAGGTTGGGG
CGATCAGCTCATCTGGACTCAGACATACGAAGAAGCTTTATACAGATCCAAGACAAGCAACAGACC
CTTGATGGTCATTCATCACTTGGACGAATGCCCACACAGTCAAGCCTTAAAGAAAGTGTTTGCTGA
ACATAAAGAAATCCAGAAATTGGCAGAGCAGTTTGTTCTCCTCAACCTGGTCTATGAAACAACCGA
CAAGCACCTTTCTCCTGATGGCCAGTACGTCCCCAGAATTGTGTTTGTAGACCCATCCCTGACGG [symbol]
GAGGGCAGACATCACTGGACGATACTCAAACCGGCTCTACGCTTATGAACCTTCTGACACAGCTTT
GTTGTACGACAACATGAAGAAAGCTCTCAAGCTGCTAAAGACAGAATTGTAGAGCTAACTGCGCAC
CGGGTCAGGAGACCAGAAGGCAGAAGCACTGTGGACTTGCAGATTACAGTACAGTTTAATGTTACA
ACAGATATATTTTTTAAACACCCACAGGTGGGGAAACAATATTATTATCTACTACAGTGAAGCATG
ATTTTCTAGAAATAAAGTCTTGTGAGAACTCCAAAAAAAAAAAAAAAAAAAAAA

Start and stop-codons are underlined. The mutated base is boxed; the wild type-sequence carries a T at the boxed position.

SEQ ID NO:2 amino acid sequence (aa) of mutant Agr2
(mus musculus)
MEKFSVSAILLLVAISGTLAKDTTVKSGAKKDPKDSRPKLPQTLSRGWGDQLIWTQTYEEALYRSK
TSNRPLMVIHHLDECPHSQALKKVFAEHKEIQKLAEQFVLLNLVYETTDKHLSPDGQYVPRIVFVD
PSLT [symbol] RADITGRYSNRLYAYEPSDTALLYDNMKKALKLLKTEL

The mutated aa is boxed; the wild type-sequence carries a V at the boxed position.

SEQ ID NO:3 amino acid sequence (aa) of wild type Agr2
(mus musculus)
MEKFSVSAILLLVAISGTLAKDTTVKSGAKKDPKDSRPKLPQTLSRGWGDQLIWTQTYEEALYRSK
TSNRPLMVIHHLDECPHSQALKKVFAEHKEIQKLAEQFVLLNLVYETTDKHLSPDGQYVPRIVFVD
PSLT [symbol] RADITGRYSNRLYAYEPSDTTALLYDNMKKALKLLKTEL

The mutated aa is boxed; the mutant-sequence carries an E at the boxed position.

SEQ ID NO:4 amino acid sequence (aa) of wild type AGR2 (human)
MEKIPVSAFLLLVALSYTLARDTTVKPGAKKDTKDSRPKLPQTLSRGWGDQLIWTQTYEEALYKSK
TSNKPLMIIHHLDECPHSQALKKVFAENKEIQKLAEQFVLLNLVYETTDKHLSPDGQYVPRIMFVD
PSLT [symbol] RADITGRYSNRLYAYEPADTALLLDNMKKALKLLKTEL

The aa corresponding to the aa mutated in mouse is boxed; a mutant-sequence would carry an E at the boxed position.

SEQ ID NO:5 nucleic acid sequence (cDNA) of human AGR2
CCGCATCCTAGCCGCCGACTCACACAAGGCAGGTGGGTGAGGAAATCCAGAGTTGCCATGGAGAAA
ATTCCAGTGTCAGCATTCTTGCTCCTTGTGGCCCTCTCCTACACTCTGGCCAGAGATACCACAGTC
AAACCTGGAGCCAAAAAGGACACAAAGGACTCTCGACCCAAACTGCCCCAGACCCTCTCCAGAGGT
TGGGGTGACCAACTCATCTGGACTCAGACATATGAAGAAGCTCTATATAAATCCAAGACAAGCAAC
AAACCCTTGATGATTATTCATCACTTGGATGAGTGCCCACACAGTCAAGCTTTAAAGAAAGTGTTT
GCTGAAAATAAAGAAATCCAGAAATTGGCAGAGCAGTTTGTCCTCCTCAATCTGGTTTATGAAACA
ACTGACAAACACCTTTCTCCTGATGGCCAGTATGTCCCCAGGATTATGTTTGTTGACCCATCTCTG
ACA [symbol] AGAGCCGATATCACTGGAAGATATTCAAATCGTCTCTATGCTTACGAACCTGCAGATACA
GCTCTGTTGCTTGACAACATGAAGAAAGCTCTCAAGTTGCTGAAGACTGAATTGTAAAGAAAAAAA
ATCTCCAAGCCCTTCTGTCTGTCAGGCCTTGAGACTTGAAACCAGAAGAAGTGTGAGAAGACTGGC
TAGTGTGGAAGCATAGTGAACACACTGATTAGGTTATGGTTTAATGTTACAACAACTATTTTTTAA
GAAAAACAAGTTTTAGAAATTTGGTTTCAAGTGTACATGTGTGAAAACAATATTGTATACTACCAT
AGTGAGCCATGATTTTCTAAAAAAAAAAATAAATGTTTTGGGGGTGTTCTGTTTTCTCCAACTTGG
TCTTTCACAGTGGTTCGTTTACCAAATAGGATTAAACACACACAAAATGCTCAAGGAAGGGACAAG
ACAAAACCAAAACTAGTTCAAATGATGAAGACCAAAGACCAAGTTATCATCTCACCACACCACAGG
TTCTCACTAGATGACTGTAAGTAGACACGAGCTTAATCAACAGAAGTATCAAGCCATGTGCTTTAG
CATAAAAGAATATTTAGAAAAACATCCCAAGAAAATCACATCACTACCTAGAGTCAACTCTGGCCA
GGAACTCTAAGGTACACACTTTCATTTAGTAATTAAATTTTAGTCAGATTTTGCCCAACCTAATGC
TCTCAGGGAAAGCCTCTGGCAAGTAGCTTTCTCCTTCAGAGGTCTAATTTAGTAGAAAGGTCATCC
AAAGAACATCTGCACTCCTGAACACACCCTGAAGAAATCCTGGGAATTGACCTTGTAATCGATTTG
TCTGTCAAGGTCCTAAAGTACTGGAGTGAAATAAATTCAGCCAACATGTGACTAATTGGAAGAAGA
GCAAAGGGTGGTGACGTGTTGATGAGGCAGATGGAGATCAGAGGTTACTAGGGTTTAGGAAACGTG
AAAGGCTGTGGCATCAGGGTAGGGGAGCATTCTGCCTAACAGAAATTAGAATTGTGTGTTAATGTC
TTCACTCTATACTTAATCTCACATTCATTAATATATGGAATTCCTCTACTGCCCAGCCCCTCCTGA
TTTCTTTGGCCCCTGGACTATGGTGCTGTATATAATGCTTTGCAGTATCTGTTGCTTGTCTTGATT
AACTTTTTTGGATAAAACCTTTTTTGAACAGAAAAAAAAAAAAAAAAAAAA

Start and stop-codons are underlined. The codon encoding valin at position 137 of the protein sequence is boxed. The point mutation to underline!

SEQ ID NO:6 nucleic acid sequence (cDNA) of wild type Agr2
(mus musculus ; C3H)
GGCAACCCTTGCGGCTCACACAAAGCAGGAGGGTGGGAAGCCCAGATTTGCCATGGAGAAATTTTC
AGTGTCTGCAATCCTGCTTCTTGTGGCCATTTCTGGTACCTTGGCCAAAGACACCACAGTCAAATC
TGGAGCCAAAAAGGACCCAAAGGACTCTCGGCCCAAACTACCTCAGACACTCTCCAGAGGTTGGGG
CGATCAGCTCATCTGGACTCAGACATACGAAGAAGCTTTATACAGATCCAAGACAAGCAACAGACC
CTTGATGGTCATTCATCACTTGGACGAATGCCCACACAGTCAAGCCTTAAAGAAAGTGTTTGCTGA
ACATAAAGAAATCCAGAAATTGGCAGAGCAGTTTGTTCTCCTCAACCTGGTCTATGAAACAACCGA
CAAGCACCTTTCTCCTGATGGCCAGTACGTCCCCAGAATTGTGTTTGTAGACCCATCCCTGACGg [symbol]
GAGGGCAGACATCACTGGACGATACTCAAACCGGCTCTACGCTTATGAACCTTCTGACACAGCTTT
GTTGTACGACAACATGAAGAAAGCTCTCAAGCTGCTAAAGACAGAATTGTAGAGCTAACTGCGCAC
CGGGTCAGGAGACCAGAAGGCAGAAGCACTGTGGACTTGCAGATTACAGTACAGTTTAATGTTACA
ACAGATATATTTTTTAAACACCCACAGGTGGGGAAACAATATTATTATCTACTACAGTGAAGCATG
ATTTTCTAGAAAATAAAGTCTTGTGAGAACTCCAAAAAAAAAAAAAAAAAAAAAA

Start and stop-codons are underlined. The mutated base is boxed; the mutant-sequence carries an A at the boxed position.

SEQ ID NO:7 mAgr2-7 primer (artificial)
5′- CAGACCCTTGATGGTCATTC -3′
SEQ ID NO:8 mAgr2-2 primer (artificial)
5′- GTCTCCTGACCCGGTGCGCAG -3′
SEQ ID NO:9 hAGR2-1 primer (artificial)
5′- GAACCTGCAGATACAGCTCTG -3′
SEQ ID NO:10 hAGR2-4 primer (artificial)
5′- CACACTAGCCAGTCTTCTCAC -3′
SEQ ID NO:11 idb2-SNP-marker (mus musculus)
CTAAACTGCGTTTCTCTCCCAATCTTTTGCAGGCATTTGGGGACTTTTTC
TTTTCTTTTTACTTTCTCTTTTTCTTTTGCACAAGAAGAAGTCTACAAGA
TCTTTTAAGACTTTTGTTATCAGCCATTTCACCAGGAGAACACGTTGAAT
GGACCTTTTTAAAAAGAAAGCGGAAGGAAAACTAAGGATGATCGTCTTGC
CCAGGTGTCTTGTTCTCCGGCCTGGACTGTGATACCGTTATTTATGAGAG
ACTTTCAGTGCCCTTTCTACAGTTGGAAGGTTTTCTTTATATACTATTCC
CACCATGGGGAGCGAAAA[G/C]GTTAAAAAAAAAAGAAAAAAATCACAA
GGAATTGCCCAATGTAAGCAGACTTTGCCTTTTCACAAAGGTGGAGCGTG
AATTCCAGAAGGACCCAGTATTCGGTTACTTAAATGAAGTCTTCGGTCAG
AAATGGCCTTTTTGACACGAGCCTACTGAATGCTGTGTATATATTTATAT
ATAAATATATATATATTGAGTGAACCTTGTGGACTCTTTAATTAGAGTTT
TCTTGTATAGTGGCAGAAATAACCTATTTCTGCATTAAAATGTAATGACG
TACTTATGCTAAACTTTTTATAAAAGTTTAGTTGTAAACTTAACCCTTTT
ATACAAAATAAATCAAGTGTGTTTATTGAATGTTGATTGCTTGCTTTATT
TCAGAC

A SNP position is underlined

SEQ ID NO:12 idb2-forward primer (artificial)
5′-CTAAACTGCGTTTCTCTCCCAA-3′
SEQ ID NO:13 idb2-reverse primer (artificial)
5′-GTCTGAAATAAAGCAAGCAATCAAC-3′
SEQ ID NO:14 D12Mit64 MIT-marker (mus musculus)
ACGNCTCACTATAGGGCGAATTGGGCCCTCTAGATGCATGCTCGAGNNGG
CCGCCAGTGTGCTGGAAAGCCTCCTTGAGATCTGAACACTTGTGTGTGTG
TGTGTGTGTGTGTGTGTGTGTGTGTGTGTGTATGTATATGTGTATAATTA
TTATTATTAGGGATTGAATCTAGGTAGACATTCTACCACAGAGACAAACC
ACCAGCCCTGCTCCTCAAATCCTTACCTCAATTTCTTTTTTTCTTTTTTT
TTGTTTTAACCTTCTCTTTTTTTATTAGATATTGTCTTCATTTACATTTC
AAATGCTATCCCAAAAG

Primer positions are underlined

SEQ ID NO:15
D12Mit64-forward primer (artificial)
5′-CTCCTTGAGATCTGAACACTTGT-3′
SEQ ID NO:16
D12Mit64-reverse primer (artificial)
5′-GGGCTGGTGGTTTGTCTCT-3′
SEQ ID NO:17

agr2-1 primer (artificial)
5′-GGATAGACCACGGATGGATA-3′

SEQ ID NO:18
agr2-2 primer (artificial)
5′-CCCCAGAGAGAACCTGATTA-3′
SEQ ID NO:19
agr2-3 primer (artificial)
5′-GTTCTCTCTGGGGGCTTTT-3′
SEQ ID NO:20
agr2-4 primer (artificial)
5′-AAGATGAGTGAGCCAAACCA-3′
SEQ ID NO:21
agr2-5 primer (artificial)
5′-GGAGTGAAGGCAGTCAACAG-3′
SEQ ID NO:22
agr2-6 primer (artificial)
5′-GATGGGACTTGGAGGAGATT-3′
SEQ ID NO:23
agr2-7 primer (artificial)
5′-TCTGTAGCCCCCTCTCTCTT-3′
SEQ ID NO:24
agr2-8 primer (artificial)
5′-CACTAAGTCCCACCGAGAAA-3′
SEQ ID NO:25
agr2-9 primer (artificial)
5′-GCTGGGGTAGGAGATAGGAG-3′
SEQ ID NO:26
agr2-10 primer (artificial)
5′-ATCTTGCCCAACTTCAGTCA-3′
SEQ ID NO:27
agr2-11 primer (artificial)
5′-TAAGCAGGAAGCAGGAGAGA-3′
SEQ ID NO:28
agr2-12 primer (artificial)
5′-AATATTGTTTCCCCACCTGT-3′
SEQ ID NO:29
nucleic acid sequence (cDNA) of mutant human AGR2
CCGCATCCTAGCCGCCGACTCACACAAGGCAGGTGGGTGAGGAAATCCAGAGTTGCCATGGAGAAA
ATTCCAGTGTCAGCATTCTTGCTCCTTGTGGCCCTCTCCTACACTCTGGCCAGAGATACCACAGTC
AAACCTGGAGCCAAAAAGGACACAAAGGACTCTCGACCCAAACTGCCCCAGACCCTCTCCAGAGGT
TGGGGTGACCAACTCATCTGGACTCAGACATATGAAGAAGCTCTATATAAATCCAAGACAAGCAAC
AAACCCTTGATGATTATTCATCACTTGGATGAGTGCCCACACAGTCAAGCTTTAAAGAAAGTGTTT
GCTGAAAATAAAGAAATCCAGAAATTGGCAGAGCAGTTTGTCCTCCTCAATCTGGTTTATGAAACA
ACTGACAAACACCTTTCTCCTGATGGCCAGTATGTCCCCAGGATTATGTTTGTTGACCCATCTCTG
ACA [symbol] AGAGCCGATATCACTGGAAGATATTCAAATCGTCTCTATGCTTACGAACCTGCAGATACA
GCTCTGTTGCTTGACAACATGAAGAAAGCTCTCAAGTTGCTGAAGACTGAATTGTAAAGAAAAAAA
ATCTCCAAGCCCTTCTGTCTGTCAGGCCTTGAGACTTGAAACCAGAAGAAGTGTGAGAAGACTGGC
TAGTGTGGAAGCATAGTGAACACACTGATTAGGTTATGGTTTAATGTTACAACAACTATTTTTTAA
GAAAAACAAGTTTTAGAAATTTGGTTTCAAGTGTACATGTGTGAAAACAATATTGTATACTACCAT
AGTGAGCCATGATTTTCTAAAAAAAAAAATAAATGTTTTGGGGGTGTTCTGTTTTCTCCAACTTGG
TCTTTCACAGTGGTTCGTTTACCAAATAGGATTAAACACACACAAAATGCTCAAGGAAGGGACAAG
ACAAAACCAAAACTAGTTCAAATGATGAAGACCAAAGACCAAGTTATCATCTCACCACACCACAGG
TTCTCACTAGATGACTGTAAGTAGACACGAGCTTAATCAACAGAAGTATCAAGCCATGTGCTTTAG
CATAAAAGAATATTTAGAAAAACATCCCAAGAAAATCACATCACTACCTAGAGTCAACTCTGGCCA
GGAACTCTAAGGTACACACTTTCATTTAGTAATTAAATTTTAGTCAGATTTTGCCCAACCTAATGC
TCTCAGGGAAAGCCTCTGGCAAGTAGCTTTCTCCTTCAGAGGTCTAATTTAGTAGAAAGGTCATCC
AAAGAACATCTGCACTCCTGAACACACCCTGAAGAAATCCTGGGAATTGACCTTGTAATCGATTTG
TCTGTCAAGGTCCTAAAGTACTGGAGTGAAATAAATTCAGCCAACATGTGACTAATTGGAAGAAGA
GCAAAGGGTGGTGACGTGTTGATGAGGCAGATGGAGATCAGAGGTTACTAGGGTTTAGGAAACGTG
AAAGGCTGTGGCATCAGGGTAGGGGAGCATTCTGCCTAACAGAAATTAGAATTGTGTGTTAATGTC
TTCACTCTATACTTAATCTCACATTCATTAATATATGGAATTCCTCTACTGCCCAGCCCCTCCTGA
TTTCTTTGGCCCCTGGACTATGGTGC
TGTATATAATGCTTTGCAGTATCTGTTGCTTGTCTTGATTAACTTTTTTGGATAAAACCTTTTTTG
AACAGAAAAAAAAAAAAAAAAAAAA

Start and stop-codons are underlined. The codon encoding valin at position 137 of the protein sequence is boxed. The codon GAR stands for either GAA or GAG, each encoding valin.

SEQ ID NO:30 amino acid sequence (aa) of human mutant AGR2
MEKIPVSAFLLLVALSYTLARDTTVKPGAKKDTKDSRPKLPQTLSRGWGDQLIWTQTYEEALYKSK
TSNKPLMIIHHLDECPHSQALKKVFAENKEIQKLAEQFVLLNLVYETTDKHLSPDGQYVPRIMFVD
PSLT [symbol] RADITGRYSNRLYAYEPADTALLLDNMKKALKLLKTEL

The aa corresponding to the aa mutated in human is boxed; the wild type-sequence carries a V at the boxed position, instead of the E indicated.

SEQ ID NO:31 humanagr2-3 primer (artificial)
5′-GCCATGGAGAAAATTCCAGTGTC-3′
SEQ ID NO:32 humanagr2-4 primer (artificial)
5′-tttacaattcagtcttcagcaacttg-3′
SEQ ID NO:33 PCR product (human)
CCATGGAGAAAATTCCAGTGTCAGCATTCTTGCTCCTTGTGGCCCTCTCC
TACACTCTGGCCAGAGATACCACAGTCAAACCTGGAGCCAAAAAGGACAC
AAAGGACTCTCGACCCAAACTGCCCCAGACCCTCTCCAGAGGTTGGGGTG
ACCAACTCATCTGGACTCAGACATATGAAGAAGCTCTATATAAATCCAAG
ACAAGCAACAAACCCTTGATGATTATTCATCACTTGGATGAGTGCCCACA
CAGTCAAGCTTTAAAGAAAGTGTTTGCTGAAAATAAAGAAATCCAGAAAT
TGGCAGAGCAGTTTGTCCTCCTCAATCTGGTTTATGAAACAACTGACAAA
CACCTTTCTCCTGATGGCCAGTATGTCCCCAGGATTATGTTTGTTGACCC
ATCTCTGACAGTTAGAGCCGATATCACTGGAAGATATTCAAATCGTCTCT
ATGCTTACGAACCTGCAGATACAGCTCTGTTGCTTGACAACATGAAGAAA
GCTCTCAAGTTGCTGAAGACTGAATTGTAAA

1-207. (canceled)

208. An isolated protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively, or an isolated fragment of such protein comprising at least 6, 7, 8, 9, 10, 15, 20, 25, 30, 35, 40, 50, 60, 70, 80, 90, 100, 110, 120, 130, 140, 150, 160, 165, 170, 171, 172, 173, or 174 contiguous amino acids having said percentages of amino acid identity compared to the corresponding amino acids in SEQ ID NO:3 and SEQ ID NO:4, wherein said protein or fragment of such protein comprises an amino acid or an amino acid sequence which corresponds to
(a) a mutation in the mouse Agr2 protein as defined above which, if encoded by the mouse Agr2 gene and present in the genome of all or essentially all cells of a mouse in a homozygous manner, results in a phenotype associated with an alteration in goblet cell function compared to the corresponding wild-type animal; and/or
(b) a mutation in the mouse Agr2 protein or the human AGR2 protein as defined above which leads to an altered biological activity of the mutated protein when compared to the corresponding wild-type mouse Agr2 protein or human AGR2 protein in an in vitro assay selected from the group consisting of a colon cell proliferation assay, a goblet cell mucus secretion assay, and a Xenopus laevis cement gland differentiation assay; and/or
(c) a mutation of the human AGR2 protein as defined above which is indicative of an increased risk of a human subject of developing a medical condition associated with an alteration in goblet cell function, or indicative of an association of a medical condition in a human subject which is associated with an alteration in goblet cell function with altered AGR2 expression or function.

209. The isolated protein or protein fragment according to claim 208, wherein said protein represents an orthologue of the mouse Agr2 or the human AGR2 protein, preferably a vertebrate orthologue, in particular an orthologue wherein said vertebrate is Xenopus leavis, or a mammalian orthologue, in particular an orthologue wherein said vertebrate is selected from the group consisting of a mouse, rat, rabbit, hamster, dog, cat, sheep, and horse.

210. The isolated protein or protein fragment according to claim 208, wherein said alteration results in a loss of function phenotype.

211. The isolated protein or protein fragment according to claim 208, wherein said alteration results in a gain of function phenotype.

212. The isolated protein or protein fragment according to claim 208, wherein said alteration is an alteration in goblet cell differentiation, particularly terminal differentiation and/or goblet cell mucus production or secretion and/or mucus composition.

213. The isolated protein or protein fragment according to claim 208, wherein said alteration is characterized by a reduction in pre-mucin storing granules in the goblet cells, an altered mucus secretion, secondary inflammatory infiltrations in the intestinal mucosal epithelium and submucosa.

214. The isolated protein or protein fragment according to claim 208, wherein said phenotype is furthermore associated with an increased proliferation of the glandular epithelium of the Brunner's gland.

215. The isolated protein or protein fragment according to claim 208, wherein said alteration results in diarrhea, or diarrhea and a thriving deficit.

216. The isolated protein or protein fragment according to claim 208, wherein said medical condition is selected from the group consisting of asthma, chronic obstructive pulmonary disease (COPD), cystic fibrosis, dry eye syndrome, gastric disease, peptic ulcer, inflammatory bowel disease, in particular Crohn's disease or ulcerative colitis, and intestinal cancer.

217. The isolated protein or protein fragment according to claim 208, wherein said mutation results in a deletion or substitution by another amino acid of an amino acid of said mouse Agr2 protein or human AGR2 protein, or an insertion of additional amino acids not normally present in the amino acid sequence of said mouse Agr2 protein or said human AGR2 protein.

218. The isolated protein or protein fragment according to claim 217, wherein the substitution of said amino acid of said mouse Agr2 protein or said human AGR2 protein by another amino acid is a non-conservative substitution.

219. The isolated protein or protein fragment according to claim 217, wherein the amino acid of said mouse Agr2 protein or said human AGR2 protein that is deleted or substituted is Val 137.

220. The isolated protein or protein fragment according to claim 219, wherein the substitution at position 137 is one of the following substitutions:
a) Val→acidic amino acid such as Glu or Asp;
b) Val→basic amino acid, such as His, Arg or Lys;
c) Val→aliphatic hydroxyl side chain amino acid, such as Ser or Thr;
d) Val→amide side chain amino acid, such as Asn or Gln;
e) Val→sulfur containing side chain amino acid, such as Cys or Met;
f) Val→aromatic side chain amino acid, such as Phe, Tyr, Trp;
g) Val→Gly or Pro; and
h) Val→Ala, Leu or Ile.

221. The isolated protein or protein fragment according to claim 220, wherein the substitution at position 137 is a substitution of valine by glutamic acid.

222. An isolated protein having the amino acid sequence set forth in SEQ ID NO:2 or SEQ ID NO:30, or an isolated fragment of such protein comprising at least 6, 7, 8, 9, 10, 15, 20, 25, 30, 35, 40, 50, 60, 70, 80, 90, 100, 110, 120, 130, 140, 150, 160, 165, 170, 171, 172, 173, or 174 contiguous amino acids of said amino acid sequence, said contiguous amino acids comprising an amino acid corresponding to Glu 137.

223. A fusion protein comprising a protein or protein fragment according to claim 208 fused to another protein or protein fragment not having said percentages of amino acid sequence identity to any corresponding amino acids in SEQ ID NO:3 and SEQ ID NO:4.

224. The fusion protein of claim 223, wherein said other protein is a protein unrelated to the mouse Agr2 or the human AGR2 protein according to SEQ ID NO:3 and SEQ ID NO:4, respectively.

225. An isolated nucleic acid encoding a protein or a fragment of such protein according to claim 208, or an isolated nucleic acid which is complementary thereto.

226. An isolated nucleic acid having the nucleotide sequence set forth in SEQ ID NO:1 or SEQ ID NO:29, or an isolated nucleic acid which is complementary thereto.

227. An episomal element comprising a nucleic acid as defined in claim 225.

228. The episomal element according to claim 227, wherein said episomal element is selected from a plasmid, a cosmid, a bacterial phage nucleic acid, or a viral nucleic acid.

229. A vector comprising a nucleic acid molecule encoding the protein according to claim 208.

230. A host cell transfected with the episomal element of claim 227.

231. A host cell transfected with the vector of claim 229.

232. An antisense nucleic acid comprising a nucleotide sequence which is complementary to
(i) a part of an mRNA encoding a protein according to claim 208, said part encoding an amino acid sequence comprising the amino acid or amino acid sequence which corresponds to
(a) the mutation in the mouse Agr2 protein according to SEQ ID NO:3 which, if encoded by the mouse Agr2 gene and present in the genome of all or essentially all cells of a mouse in a homozygous manner, results in a phenotype associated with an alteration in goblet cell function compared to the corresponding wild-type animal, said phenotype optionally being furthermore associated with an increased proliferation of the glandular epithelium of the Brunner's gland; and/or
(b) the mutation in the mouse Agr2 protein or the human AGR2 protein according to SEQ ID NO:3 and SEQ ID NO:4, respectively, which leads to an altered biological activity of the mutated protein when compared to the corresponding wild-type mouse Agr2 protein or human AGR2 protein in an in vitro assay selected from the group consisting of a colon cell proliferation assay, a goblet cell mucus secretion assay, and a Xenopus laevis cement gland differentiation assay; and/or
(c) the mutation of the human AGR2 protein according to SEQ ID NO:4 which is indicative of an increased risk of a human subject of developing a medical condition associated with an alteration in goblet cell function, or indicative of an association of a medical condition in a human subject which is associated with an alteration in goblet cell function with altered AGR2 expression or function;
(ii) a part of the mRNA encoding the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein as defined above, said part being a non-coding part and comprising a sequence corresponding to a mutation in the gene coding for said protein or orthologue which affects expression of said protein or orthologue; or
(iii) a part of the mRNA encoding a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

233. The antisense nucleic acid of claim 232, wherein said antisense nucleic acid is capable of hybridizing to said mRNA via said complementary nucleotide sequence under physiological conditions, or under conditions of high stringency, preferably under hybridization conditions of a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C., furthermore preferably under hybridization conditions of a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 65° C.

234. The antisense nucleic acid of claim 233, wherein said hybridization to said mRNA is more effective than hybridization to
(i) the mRNA encoding the same protein which, however, corresponds to the wild-type mouse Agr2 or human AGR2 protein according to SEQ ID NO:3 and SEQ ID NO:4 in respect of said amino acid sequence;
(ii) the mRNA encoded by the wild-type gene of the mouse Agr2 or human AGR2 protein as defined above, or the wild-type gene of the corresponding orthologue; or
(iii) the mRNA encoded by the wild-type gene of the corresponding protein which affects expression or function of the mouse Agr2 or the human AGR2 protein as defined above.

235. A host cell transformed with an antisense nucleic acid according to claim 232.

236. The host cell according to claim 235, wherein said host cell is a eukaryotic cell.

237. The host cell according to claim 235, wherein said host cell is a prokaryotic cell.

238. A short interfering RNA (siRNA) comprising a double stranded nucleotide sequence wherein one strand is complementary to an at least 19, 20, 21, 22, 23, 24, or 25 nucleotide long segment of an mRNA encoding
(a) the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or
(b) a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

239. The siRNA of claim 238, wherein said siRNA is capable of silencing or suppressing the expression of the AGR2 gene encoding said mRNA.

240. The siRNA of claim 238, wherein said AGR2 gene is an AGR2 gene of a human subject unaffected by or known not to be at risk of developing a condition associated with an alteration in goblet cell function.

241. The siRNA according to claim 238, wherein said segment includes sequences from the 5′ untranslated (UT) region, the open reading frame (ORF), or the 3′ UT region of said mRNA.

242. A host cell transformed with an siRNA according to claim 238.

243. The host cell according to claim 242, wherein said host cell is a eukaryotic cell.

244. The host cell according to claim 242, wherein said host cell is a prokaryotic cell.

245. An anticalin specifically binding an epitope in a protein which corresponds to
(a) the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or
(b) a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

246. An aptamer specifically binding an epitope in a protein which corresponds to
(a) the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or
(b) a protein which affects expression or function of the mouse Agr2 or the human AGR2 protein according to SEQ ID NO:3 and SEQ ID NO:4, respectively, or an orthologue thereof having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively.

247. A non-human vertebrate animal comprising in the genome of at least some of its cells an allele of a gene encoding a protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively, said allele comprising a mutation which,
a) if present in the genome of all or essentially all cells of said animal in a homozygous manner, results in a phenotype associated with an alteration in goblet cell function compared to the corresponding wild-type animal; and/or
b) corresponds to a mutation in the mouse Agr2 protein or the human AGR2 protein as defined above which leads to an altered biological activity of the mutated protein when compared to the corresponding wild-type mouse Agr2 protein or human AGR2 protein in an in vitro assay selected from the group consisting of a colon cell proliferation assay, a goblet cell mucus secretion assay, and a Xenopus laevis cement gland differentiation assay; and/or
c) corresponds to a mutation of the human AGR2 protein as defined above which is indicative of an increased risk of a human subject of developing a medical condition associated with an alteration in goblet cell function, or indicative of an association of a medical condition in a human subject which is associated with an alteration in goblet cell function with altered AGR2 expression or function.

248. A non-human vertebrate animal comprising in the genome of at least some of its cells an allele of a gene coding for a protein which affects expression or function of the AGR2 protein of said animal, said allele comprising a mutation which, if present in the genome of all or essentially all cells of said animal in a homozygous manner, results in a phenotype associated with an alteration in goblet cell function compared to the corresponding wild-type animal.

249. The animal according to claim 247, wherein said alteration results in a loss of function phenotype.

250. The animal according to claim 247, wherein said alteration results in a gain of function phenotype.

251. The animal according to claim 247, wherein said alteration is an alteration in goblet cell differentiation, particularly terminal differentiation, and/or goblet cell mucus production or secretion and/or mucus composition.

252. The animal according to claim 247, wherein said alteration is characterized by a reduction in pre-mucin storing granules in the goblet cells, an altered mucus secretion, and secondary inflammatory infiltrations in the intestinal mucosal epithelium and submucosa.

253. The animal according to claim 247, wherein said phenotype is furthermore associated with an increased proliferation of the glandular epithelium of the Brunner's gland.

254. The animal according to claim 247, wherein said alteration results in diarrhea, or diarrhea and a thriving deficit.

255. The animal according to claim 247, wherein said gene encodes a protein which is an orthologue of SEQ ID NO:3 and SEQ ID NO:4 with respect to said animal.

256. The animal according to claim 247, wherein said gene encodes a protein according to claim 208.

257. The animal according to claim 247, wherein said gene encodes a protein having the amino acid sequence of SEQ ID NO:2 or SEQ ID NO:30.

258. The animal according to claim 247, wherein said animal is a transgenic animal.

259. The animal according to claim 247, wherein said cells are the germ cells of said animal.

260. The animal according to claim 247, wherein said cells are the somatic cells of said animal.

261. The animal according to claim 247, wherein said genome of said cells is homozygous in respect of said allele.

262. The animal according to claim 247, wherein said animal is a mammalian animal, preferably a rodent.

263. The animal according to claim 262, wherein said animal is selected from the group consisting of a mouse, rat, rabbit, hamster, dog, cat, sheep, and horse.

264. A method for the identification of a protein or nucleic acid diagnostic marker for a goblet cell-related disorder, or as an animal model for studying the molecular mechanisms of, or physiological processes associated with, a goblet cell-related disorder, or for the identification and testing of an agent useful in the prevention, amelioration, or treatment of a goblet cell-related disorder comprising administering said agent to the non-human vertebrate animal of claim 247 and measuring or monitoring a phenotypic parameter in said animal.

265. The method according to claim 264, wherein said goblet cell-related disorder is selected from the group consisting of asthma, chronic obstructive pulmonary disease (COPD), cystic fibrosis, dry eye syndrome, gastric disease, peptic ulcer, inflammatory bowel disease, in particular Crohn's disease or ulcerative colitis, and intestinal cancer.

266. A method for studying the molecular mechanisms of, or physiological processes associated with, conditions associated with, or affected by, reduced activity or undesirable, e.g. increased, activity of endogenous AGR2; reduced expression, reduced production or undesirable, e.g. increased, production of endogenous AGR2; or for the identification and testing of an agent useful in the prevention, amelioration, or treatment of these conditions comprising administering said agent to the non-human vertebrate animal of claim 247 and measuring or monitoring a phenotypic parameter in said animal.

267. The method according to claim 264, wherein said agent is selected from the group consisting of a small molecule drug, a (poly)peptide, and a nucleic acid.

268. The method according to claim 266, wherein said agent is selected from the group consisting of a small molecule drug, a (poly)peptide, and a nucleic acid.

269. The agent of claim 267, wherein said agent is an antagonist of AGR2.

270. The agent of claim 268, wherein said agent is an antagonist of AGR2.

271. The agent of claim 267, wherein said agent is an agonist of AGR2.

272. The agent of claim 268, wherein said agent is an agonist of AGR2.

273. A method for studying or identifying protein or nucleic acid diagnostic markers, such as an early gene diagnostic marker, for diseases associated with AGR2 deficiency or over-expression comprising subjecting an organ or tissue of the non-human vertebrate animal according to claim 247 to procedures of proteomics or gene expression analysis.

274. A method of identifying
(a) a protein or nucleic acid marker indicative of an increased risk of a human subject of developing a medical condition associated with an alteration in goblet cell function; or
(b) a protein or nucleic acid marker indicative of an association of a medical condition in a human subject which is associated with an alteration in goblet cell function with altered AGR2 expression or function
said method comprising the step of analyzing a test sample derived from a human subject for the presence of a difference compared to a similar test sample if derived from a human subject unaffected by or known not to be at risk of developing said condition, wherein said difference is indicative of the presence of a mutation in an allele of the gene coding for the AGR2 protein according to SEQ ID NO:4, or in an allele of a gene coding for a protein which affects expression or function of said AGR2 protein.

275. The method of claim 274, wherein said test sample is analyzed for a difference compared to similar test samples if derived from a group of human subjects unaffected by, or known not to be at risk of developing, said condition.

276. The method according to claim 274, wherein said human subject whose test sample is analyzed has a condition or is known or suspected to be at risk of developing a condition associated with an alteration in goblet cell function.

277. The method of claim 274, further comprising the step of obtaining said similar test sample from said human subject unaffected by, or known not to be at risk of developing, said condition.

278. The method according to claim 274, wherein said alteration is an alteration in goblet cell differentiation, particularly terminal differentiation, and/or goblet cell mucus production or secretion and/or mucus composition.

279. The method according to claim 274, wherein said alteration is characterized by a reduction in pre-mucin storing granules in the goblet cells, an altered mucus secretion, and secondary inflammatory infiltrations in the intestinal mucosal epithelium and submucosa.

280. The method according to claim 274, wherein said medical condition is furthermore associated with an increased proliferation of the glandular epithelium of the Brunner's gland.

281. The method according to claim 274, wherein said alteration results in diarrhea.

282. The method according to claim 274, wherein said medical condition is selected from the group consisting of asthma, chronic obstructive pulmonary disease (COPD), cystic fibrosis, dry eye syndrome, gastric disease, peptic ulcer, inflammatory bowel disease, in particular Crohn's disease or ulcerative colitis, and intestinal cancer.

283. The method according to claim 274, wherein said medical condition is associated with an increase in mucus production.

284. The method according to claim 274, wherein said test sample is a nucleic acid sample.

285. The method according to claim 284, wherein the step of analyzing said nucleic acid sample comprises amplifying at least a portion of its nucleic acid via the polymerase chain reaction, and optionally also amplifying via the polymerase chain reaction at least a portion of the nucleic acid of said similar sample or said similar samples.

286. The method according to claim 274, wherein said test sample is a protein sample.

287. The method according to claim 286, wherein said protein is the AGR2 protein.

288. The method according to claim 274, wherein said mutation results in a deletion or substitution by another amino acid of an amino acid of the AGR2 protein encoded by said allele, or an insertion of additional amino acids not normally present in the amino acid sequence of the AGR2 protein according to SEQ ID NO:4.

289. The method according to claim 288, wherein the substitution of said amino acid of the AGR2 protein by another amino acid is a non-conservative substitution.

290. The method according to claim 288, wherein said amino acid of the AGR2 protein that is deleted or substituted is Val 137.

291. The method according to claim 290, wherein the substitution at position 137 is one of the following substitutions:
a) Val→acidic amino acid such as Glu or Asp;
b) Val→basic amino acid, such as His, Arg or Lys;
c) Val→aliphatic hydroxyl side chain amino acid, such as Ser or Thr;
d) Val→amide side chain amino acid, such as Asn or Gln;
e) Val→sulfur containing side chain amino acid, such as Cys or Met;
f) Val→aromatic side chain amino acid, such as Phe, Tyr, Trp;
g) Val→Gly or Pro; and
h) Val→Ala, Leu or Ile.

292. The method according to claim 291, wherein the substitution at position 137 is a substitution of valine by glutamic acid.

293. A method for identifying a predisposition of a human subject for developing a medical condition associated with an alteration in goblet cell function, said method comprising the step of determining whether a test sample derived from said human subject indicates the presence of a mutation in an allele of the gene coding for the AGR2 protein according to SEQ ID NO:4 indicative of an increased risk of said human subject of developing said medical condition.

294. The method according to claim 293, further comprising the step of assigning a certain risk of developing said medical condition to said human subject.

295. A method for determining whether a medical condition in a human subject which is associated with an alteration in goblet cell function is associated with altered AGR2 expression or function, said method comprising the step of determining whether a test sample derived from said human subject indicates the presence of a mutation in an allele of the gene coding for the AGR2 protein according to SEQ ID NO:4 indicative of an altered AGR2 expression or function.

296. The method according to claim 295, further comprising the step of assigning an association with altered AGR2 expression or function to said human subject's medical condition.

297. The method according to claim 293, wherein said alteration is an alteration in goblet cell differentiation, particularly terminal differentiation, and/or goblet cell mucus production or secretion and/or mucus composition.

298. The method according to claim 295, wherein said alteration is an alteration in goblet cell differentiation, particularly terminal differentiation, and/or goblet cell mucus production or secretion and/or mucus composition.

299. The method according to claim 293, wherein said alteration is characterized by a reduction in pre-mucin storing granules in the goblet cells, an altered mucus secretion, and secondary inflammatory infiltrations in the intestinal mucosal epithelium and submucosa.

300. The method according to claim 295, wherein said alteration is characterized by a reduction in pre-mucin storing granules in the goblet cells, an altered mucus secretion, and secondary inflammatory infiltrations in the intestinal mucosal epithelium and submucosa.

301. The method according to claim 293, wherein said medical condition is selected from the group consisting of asthma, chronic obstructive pulmonary disease (COPD), cystic fibrosis, dry eye syndrome, gastric disease, peptic ulcer, inflammatory bowel disease, in particular Crohn's disease or ulcerative colitis, and intestinal cancer.

302. The method according to claim 293, wherein said medical condition is associated with an increase in mucus production.

303. The method according to claim 295, wherein said medical condition is selected from the group consisting of asthma, chronic obstructive pulmonary disease (COPD), cystic fibrosis, dry eye syndrome, gastric disease, peptic ulcer, inflammatory bowel disease, in particular Crohn's disease or ulcerative colitis, and intestinal cancer.

304. The method according to claim 295, wherein said medical condition is associated with an increase in mucus production.

305. The method according to claim 293, wherein said test sample is a nucleic acid sample.

306. The method according to claim 295, wherein said test sample is a nucleic acid sample.

307. The method according to claim 293, wherein said test sample is a protein sample.

308. The method according to claim 295, wherein said test sample is a protein sample.

309. The method according to claim 307, wherein said protein is the AGR2 protein.

310. The method according to claim 308, wherein said protein is the AGR2 protein.

311. The method according to claim 293, wherein said mutation results in a deletion or substitution by another amino acid of an amino acid of the AGR2 protein encoded by said allele, or an insertion of additional amino acids not normally present in the amino acid sequence of the AGR2 protein according to SEQ ID NO:4.

312. The method according to claim 295, wherein said mutation results in a deletion or substitution by another amino acid of an amino acid of the AGR2 protein encoded by said allele, or an insertion of additional amino acids not normally present in the amino acid sequence of the AGR2 protein according to SEQ ID NO:4.

313. The method according to claim 311, wherein said amino acid of the AGR2 protein that is deleted or substituted is Val 137.

314. The method according to claim 312, wherein said amino acid of the AGR2 protein that is deleted or substituted is Val 137.

315. The method according to claim 313, wherein the substitution at position 137 is one of the following substitutions:
a) Val→acidic amino acid such as Glu or Asp;
b) Val→basic amino acid, such as His, Arg or Lys;
c) Val→aliphatic hydroxyl side chain amino acid, such as Ser or Thr;
d) Val→amide side chain amino acid, such as Asn or Gln;
e) Val→sulfur containing side chain amino acid, such as Cys or Met;
f) Val→aromatic side chain amino acid, such as Phe, Tyr, Trp;
g) Val→Gly or Pro; and
h) Val→Ala, Leu or Ile.

316. The method according to claim 314, wherein the substitution at position 137 is one of the following substitutions:
i) Val→acidic amino acid such as Glu or Asp;
j) Val→basic amino acid, such as His, Arg or Lys;
k) Val→aliphatic hydroxyl side chain amino acid, such as Ser or Thr;
l) Val→amide side chain amino acid, such as Asn or Gln;
m) Val→sulfur containing side chain amino acid, such as Cys or Met;
n) Val→aromatic side chain amino acid, such as Phe, Tyr, Trp;
o) Val→Gly or Pro; and
p) Val→Ala, Leu or Ile.

317. The method according to claim 315, wherein the substitution at position 137 is a substitution of valine by glutamic acid.

318. The method according to claim 316, wherein the substitution at position 137 is a substitution of valine by glutamic acid.

319. The method according to claim 293, wherein said gene codes for a AGR2 protein having the sequence set forth in SEQ ID NO:30.

320. The method according to claim 295, wherein said gene codes for a AGR2 protein having the sequence set forth in SEQ ID NO:30.

321. A pharmaceutical composition comprising an antisense nucleic acid according to claim 232 and a pharmaceutically acceptable carrier.

322. A pharmaceutical composition comprising an siRNA according to claim 238 and a pharmaceutically acceptable carrier.

323. A pharmaceutical composition comprising an anticalin according to claim 245 and a pharmaceutically acceptable carrier.

324. A pharmaceutical composition comprising an aptamer according to claim 246 and a pharmaceutically acceptable carrier.

325. A method of producing a mutant AGR2 protein comprising culturing a host cell according to claim 230 in a suitable medium under conditions such that the protein is expressed, and harvesting the cells or the medium.

326. The method according to claim 325, wherein the protein is subsequently further purified from said cells or said medium.

327. A method of gene therapy comprising delivering to cells in a human subject suffering from or known to be at risk of developing a condition associated with an alteration in goblet cell function a DNA construct comprising
(a) a sequence of an allele of the AGR2 gene encoding the human AGR2 protein according to SEQ ID NO:4, or encoding a protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or a sequence of an allele of the AGR2 gene of a human subject unaffected by or known not to be at risk of developing said condition;
(b) a DNA sequence encoding the human AGR2 protein according to SEQ ID NO:4, or a human AGR2 protein encoded by the AGR2 gene of a human subject unaffected by or known not to be at risk of developing said condition, or a protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively; or
(c) a DNA sequence encoding an antisense nucleic acid according to claim 232, or an antisense nucleic acid comprising a nucleotide sequence which is complementary to an mRNA encoded by the AGR2 gene of a human subject unaffected by or known not to be at risk of developing said condition.

328. A method of gene therapy comprising delivering to cells in a human subject suffering from or known to be at risk of developing a condition associated with an alteration in goblet cell function a DNA construct comprising a DNA sequence encoding an siRNA according to claim 238.

329. A method of gene therapy comprising delivering to cells in a human subject suffering from or known to be at risk of developing a condition associated with an alteration in goblet cell function a DNA construct comprising a DNA sequence encoding an aptamer according claim 246.

330. The method of claim 327, wherein said human AGR2 gene of a subject unaffected by or known not to be at risk of developing said condition is a gene encoding a human AGR2 protein according to SEQ ID NO:4.

331. The method of claim 327, wherein said cells are intestinal cells of said human subject, preferably goblet cells.

332. The method of claim 328, wherein said cells are intestinal cells of said human subject, preferably goblet cells.

333. The method of claim 329, wherein said cells are intestinal cells of said human subject, preferably goblet cells.

334. The method of claim 327, wherein said cells are gastrointestinal cells of said human subject, preferably goblet cells and/or mucus secreting cells of the Brunner's gland.

335. The method of claim 328, wherein said cells are gastrointestinal cells of said human subject, preferably goblet cells and/or mucus secreting cells of the Brunner's gland.

336. The method of claim 329, wherein said cells are gastrointestinal cells of said human subject, preferably goblet cells and/or mucus secreting cells of the Brunner's gland.

337. The method of claim 327, wherein the DNA construct is a viral vector.

338. The method of claim 328, wherein the DNA construct is a viral vector.

339. The method of claim 329, wherein the DNA construct is a viral vector.

340. The method of claim 327, wherein said DNA construct is capable of directing expression of said protein, said antisense nucleic acid, or said siRNA.

341. The method of claim 328, wherein said DNA construct is capable of directing expression of said protein, said antisense nucleic acid, or said siRNA.

342. The method of claim 329, wherein said DNA construct is capable of directing expression of said protein, said antisense nucleic acid, or said siRNA.

343. The method of claim 327, wherein said sequence of an allele of the AGR2 gene comprises coding sequences of said gene.

344. The method of claim 328, wherein said sequence of an allele of the AGR2 gene comprises coding sequences of said gene.

345. The method of claim 329, wherein said sequence of an allele of the AGR2 gene comprises coding sequences of said gene.

346. A method of preventing, treating, or ameliorating a medical condition in a human subject associated with an alteration in goblet cell function, said method comprising administering to said human subject a pharmaceutical composition comprising an agent capable of modulating AGR2 activity in said human subject.

347. The method according to claim 346, wherein said medical condition is associated with an increase in mucus production.

348. The method of claim 346, wherein said pharmaceutical composition is a pharmaceutical composition according to claim 321.

349. The method of claim 346, wherein said pharmaceutical composition is a pharmaceutical composition according to claim 322.

350. The method of claim 346, wherein said pharmaceutical composition is a pharmaceutical composition according to claim 323.

351. The method of claim 346, wherein said pharmaceutical composition is a pharmaceutical composition according to claim 324.

352. The method according to claim 346, wherein said agent capable of modulating AGR2 activity in said human subject is
(a) an isolated protein having the sequence of the human AGR2 protein according to SEQ ID NO:4,
(b) an isolated protein having at least 65%, 70%, 75%, 80%, 85%, 90%, 95%, 98%, or 99% amino acid identity compared to the mouse Agr2 or the human AGR2 protein according to SEQ ID NO:3 and SEQ ID NO:4, respectively, wherein said protein shows the same or essentially the same activity as the human AGR2 protein according to SEQ ID NO:4 in an in vitro assay selected from the group consisting of a colon cell proliferation assay, a goblet cell mucus secretion assay, and a Xenopus laevis cement gland differentiation assay;
(c) an isolated fragment of the protein according to (a) or (b) above comprising at least 6, 7, 8, 9, 10, 15, 20, 25, 30, 35, 40, 50, 60, 70, 80, 90, 100, 110, 120, 130, 140, 150, 160, 165, 170, 171, 172, 173, or 174 contiguous amino acids having said percentages of amino acid identity compared to the corresponding amino acids in SEQ ID NO:3 and SEQ ID NO:4, wherein said fragment shows the same or essentially the same activity as the human AGR2 protein according to SEQ ID NO:4 in an in vitro assay selected from the group consisting of a colon cell proliferation assay, a goblet cell mucus secretion assay, and a Xenopus laevis cement gland differentiation assay;
(d) a fusion protein comprising a protein or protein fragment according to (a) to (c) above fused to another protein or protein fragment not having said percentages of amino acid sequence identity to any corresponding amino acids in SEQ ID NO:3 and SEQ ID NO:4; preferably fused to a protein unrelated to the mouse Agr2 or the human AGR2 protein according to SEQ ID NO:3 and SEQ ID NO:4, respectively;
(e) an antibody specifically recognizing an epitope comprised within the human AGR2 protein according to SEQ ID NO:4, or within a human AGR2 protein encoded by the AGR2 gene of a human subject unaffected by or known not to be at risk of developing a medical condition associated with altered goblet cell function; or
(f) an antisense nucleic acid comprising a nucleotide sequence which is complementary to an mRNA encoded by the AGR2 gene of a human subject unaffected by or known not to be at risk of developing said condition, preferably encoded by the AGR2 gene encoding the human AGR2 protein according to SEQ ID NO:4.

353. A method of identifying an agent useful in the prevention, amelioration, or treatment of a goblet cell-related disorder, the method comprising
a) culturing mammalian goblet cells in the presence or absence of a candidate agent; and
b) determining whether the presence of the agent results in an increase in the production by the cells of mucus and/or one or more particular mucus constituents;
wherein said goblet cells show a reduced or no expression of the AGR2 protein, or carry a mutation in one or both alleles of their endogenous AGR2 gene so that the allele is no longer capable of being expressed, or that it encodes a protein according to claim 208.

354. A method of identifying an agent useful in the prevention, amelioration, or treatment of a goblet cell-related disorder, the method comprising
a) culturing mammalian goblet cells in the presence or absence of a candidate agent; and
b) determining whether the presence of the agent results in a decrease in the production by the cells of mucus and/or one or more particular mucus constituents;
wherein said goblet cells show an increased expression of the AGR2 protein, or carry a mutation in one or both alleles of their endogenous AGR2 gene so that the allele shows an increased amount of expression or that it encodes a protein according to claim 208.

355. A method of identifying an antagonist of the AGR2 protein, the method comprising
a) culturing mammalian goblet cells in the presence or absence of a wild-type mammalian AGR2 protein, preferably the mouse Agr2 protein or the human AGR2 protein according to SEQ ID NO:3 and SEQ ID NO:4, respectively; and
b) determining whether a decrease in the production of mucus and/or one or more particular mucus constituents by the cells which are cultured in the presence of said wild-type AGR2 protein is observed upon the addition of a candidate antagonist agent to the cultured cells.

356. The method according to claim 355, wherein said goblet cells show a reduced or no expression of the AGR2 protein, or carry a mutation in one or both alleles of their endogenous AGR2 gene so that the allele is no longer capable of being expressed or that it encodes a protein according to claim 208.

357. The method according to claim 353, wherein said cells are homozygous for said mutated endogenous AGR2 allele.

358. The method according to claim 354, wherein said cells are homozygous for said mutated endogenous AGR2 allele.

359. The method according to claim 353, wherein said cells do not additionally contain a functional allele of a wild type AGR2 gene (i.e., no functional allele of the corresponding wild type orthologue, or of a heterologous wild type AGR2 gene), or a nucleic acid sequence expressing a wild type AGR2 protein (representing either the corresponding wild type orthologue, or a heterologous wild type AGR2 protein).

360. The method according to claim 353, wherein the mucus constituent is mucin2 or a trefoil peptide.

361. The method according to claim 354, wherein the mucus constituent is mucin2 or a trefoil peptide.

362. The method according to claim 355, wherein the mucus constituent is mucin2 or a trefoil peptide.

363. The method according to claim 353, wherein said mammalian goblet cells are LS174T or HT29 cells.

364. The method according to claim 354, wherein said mammalian goblet cells are LS174T or HT29 cells.

365. The method according to claim 355, wherein said mammalian goblet cells are LS174T or HT29 cells.

366. The method according to claim 353, wherein the candidate agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton.

367. The method according to claim 354, wherein the candidate agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton.

368. The method according to claim 355, wherein the candidate agent is selected from the group consisting of
a) a peptide or polypeptide;
b) a nucleic acid (including a peptide nucleic acid); and
c) a small molecule having a molecular weight of no more than 2000 Dalton, preferably no more than 1500 Dalton, more preferably no more than 1000 Dalton, and most preferably no more than 500, 400, 300, or even 200 Dalton.

369. An agent identified or identifiable by a method according claim 353.

370. An agent identified or identifiable by a method according claim 354.

371. An agent identified or identifiable by a method according claim 355.


SEQUENCE LISTING (34 sequences)

SEQ ID NO: 1 - DNA, 781 bp, Mus musculus
ggcaacccttgcggctcacacaaagcaggagggtgggaagcccagatttgccatggagaaattttcagtgtctgcaatcctgcttcttgtggccatttctggtaccttggccaaagacaccacagtcaaatctggagccaaaaaggacccaaaggactctcggcccaaactacctcagacactctccagaggttggggcgatcagctcatctggactcagacatacgaagaagctttatacagatccaagacaagcaacagacccttgatggtcattcatcacttggacgaatgcccacacagtcaagccttaaagaaagtgtttgctgaacataaagaaatccagaaattggcagagcagtttgttctcctcaacctggtctatgaaacaaccgacaagcacctttctcctgatggccagtacgtccccagaattgtgtttgtagacccatccctgacggagagggcagacatcactggacgatactcaaaccggctctacgcttatgaaccttctgacacagctttgttgtacgacaacatgaagaaagctctcaagctgctaaagacagaattgtagagctaactgcgcaccgggtcaggagaccagaaggcagaagcactgtggacttgcagattacagtacagtttaatgttacaacagatatattttttaaacacccacaggtggggaaacaatattattatctactacagtgaagcatgattttctagaaaataaagtcttgtgagaactccaaaaaaaaaaaaaaaaaaaaaa

SEQ ID NO: 2 - protein, 175 aa, Mus musculus
MEKFSVSAILLLVAISGTLAKDTTVKSGAKKDPKDSRPKLPQTLSRGWGDQLIWTQTYEEALYRSKTSNRPLMVIHHLDECPHSQALKKVFAEHKEIQKLAEQFVLLNLVYETTDKHLSPDGQYVPRIVFVDPSLTERADITGRYSNRLYAYEPSDTALLYDNMKKALKLLKTEL

SEQ ID NO: 3 - protein, 175 aa, Mus musculus
MEKFSVSAILLLVAISGTLAKDTTVKSGAKKDPKDSRPKLPQTLSRGWGDQLIWTQTYEEALYRSKTSNRPLMVIHHLDECPHSQALKKVFAEHKEIQKLAEQFVLLNLVYETTDKHLSPDGQYVPRIVFVDPSLTVRADITGRYSNRLYAYEPSDTALLYDNMKKALKLLKTEL

SEQ ID NO: 4 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTVRADITGRYSNRLYAYEPADTALLLDNMKKALKLLKTEL

SEQ ID NO: 5 - DNA, 1701 bp, Homo sapiens
ccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagttttagaaatttggtttcaagtgtacatgtgtgaaaacaatattgtatactaccatagtgagccatgattttctaaaaaaaaaaataaatgttttgggggtgttctgttttctccaacttggtctttcacagtggttcgtttaccaaataggattaaacacacacaaaatgctcaaggaagggacaagacaaaaccaaaactagttcaaatgatgaagaccaaagaccaagttatcatctcaccacaccacaggttctcactagatgactgtaagtagacacgagcttaatcaacagaagtatcaagccatgtgctttagcataaaagaatatttagaaaaacatcccaagaaaatcacatcactacctagagtcaactctggccaggaactctaaggtacacactttcatttagtaattaaattttagtcagattttgcccaacctaatgctctcagggaaagcctctggcaagtagctttctccttcagaggtctaatttagtagaaaggtcatccaaagaacatctgcactcctgaacacaccctgaagaaatcctgggaattgaccttgtaatcgatttgtctgtcaaggtcctaaagtactggagtgaaataaattcagccaacatgtgactaattggaagaagagcaaagggtggtgacgtgttgatgaggcagatggagatcagaggttactagggtttaggaaacgtgaaaggctgtggcatcagggtaggggagcattctgcctaacagaaattagaattgtgtgttaatgtcttcactctatacttaatctcacattcattaatatatggaattcctctactgcccagcccctcctgatttctttggcccctggactatggtgctgtatataatgctttgcagtatctgttgcttgtcttgattaacttttttggataaaaccttttttgaacagaaaaaaaaaaaaaaaaaaaa

SEQ ID NO: 6 - DNA, 781 bp, Mus musculus
ggcaacccttgcggctcacacaaagcaggagggtgggaagcccagatttgccatggagaaattttcagtgtctgcaatcctgcttcttgtggccatttctggtaccttggccaaagacaccacagtcaaatctggagccaaaaaggacccaaaggactctcggcccaaactacctcagacactctccagaggttggggcgatcagctcatctggactcagacatacgaagaagctttatacagatccaagacaagcaacagacccttgatggtcattcatcacttggacgaatgcccacacagtcaagccttaaagaaagtgtttgctgaacataaagaaatccagaaattggcagagcagtttgttctcctcaacctggtctatgaaacaaccgacaagcacctttctcctgatggccagtacgtccccagaattgtgtttgtagacccatccctgacggtgagggcagacatcactggacgatactcaaaccggctctacgcttatgaaccttctgacacagctttgttgtacgacaacatgaagaaagctctcaagctgctaaagacagaattgtagagctaactgcgcaccgggtcaggagaccagaaggcagaagcactgtggacttgcagattacagtacagtttaatgttacaacagatatattttttaaacacccacaggtggggaaacaatattattatctactacagtgaagcatgattttctagaaaataaagtcttgtgagaactccaaaaaaaaaaaaaaaaaaaaaa

SEQ ID NO: 7 - DNA, 20 bp, Artificial Sequence
  mAgr2-7 primer
cagacccttgatggtcattc

SEQ ID NO: 8 - DNA, 21 bp, Artificial Sequence
  mAgr2-2 primer
gtctcctgacccggtgcgcag

SEQ ID NO: 9 - DNA, 21 bp, Artificial Sequence
  hAgr1 primer
gaacctgcagatacagctctg

SEQ ID NO: 10 - DNA, 21 bp, Artificial Sequence
  hAgr4 primer
cacactagccagtcttctcac

SEQ ID NO: 11 - DNA, 702 bp, Mus musculus
  misc_feature (319)..(319): n is a, c, g, or t
ctaaactgcgtttctctcccaatcttttgcaggcatttggggactttttcttttctttttactttctctttttcttttgcacaagaagaagtctacaagatcttttaagacttttgttatcagccatttcaccaggagaacacgttgaatggacctttttaaaaagaaagcggaaggaaaactaaggatgatcgtcttgcccaggtgtcttgttctccggcctggactgtgataccgttatttatgagagactttcagtgccctttctacagttggaaggttttctttatatactattcccaccatggggagcgaaaangttaaaaaaaaaagaaaaaaatcacaaggaattgcccaatgtaagcagactttgccttttcacaaaggtggagcgtgaattccagaaggacccagtattcggttacttaaatgaagtcttcggtcagaaatggcctttttgacacgagcctactgaatgctgtgtatatatttatatataaatatatatatattgagtgaaccttgtggactctttaattagagttttcttgtatagtggcagaaataacctatttctgcattaaaatgtaatgacgtacttatgctaaactttttataaaagtttagttgtaaacttaacccttttatacaaaataaatcaagtgtgtttattgaatgttgattgcttgctttatttcagac

SEQ ID NO: 12 - DNA, 22 bp, Artificial Sequence
  primer 1 Idb2-SNP-marker
ctaaactgcgtttctctcccaa

SEQ ID NO: 13 - DNA, 25 bp, Artificial Sequence
  primer 2 Idb2-SNP-marker
gtctgaaataaagcaagcaatcaac

SEQ ID NO: 14 - DNA, 317 bp, Mus musculus
  misc_feature (4)..(4): n is a, c, g, or t
acgnctcactatagggcgaattgggccctctagatgcatgctcgagnnggccgccagtgtgctggaaagcctccttgagatctgaacacttgtgtgtgtgtgtgtgtgtgtgtgtgtgtgtgtgtgtgtgtatgtatatgtgtataattattattattagggattgaatctaggtagacattctaccacagagacaaaccaccagccctgctcctcaaatccttacctcaatttctttttttctttttttttgttttaaccttctctttttttattagatattgtcttcatttacatttcaaatgctatcccaaaag

SEQ ID NO: 15 - DNA, 23 bp, Artificial Sequence
  primer 1 D12Mit64 MIT-marker
ctccttgagatctgaacacttgt

SEQ ID NO: 16 - DNA, 19 bp, Artificial Sequence
  primer 2 D12Mit64 MIT-marker
gggctggtggtttgtctct

SEQ ID NO: 17 - DNA, 20 bp, Artificial Sequence
  Agr2 primer 1
ggatagaccacggatggata

SEQ ID NO: 18 - DNA, 20 bp, Artificial Sequence
  Agr2 primer 2
ccccagagagaacctgatta

SEQ ID NO: 19 - DNA, 19 bp, Artificial Sequence
  Agr2 primer 3
gttctctctgggggctttt

SEQ ID NO: 20 - DNA, 20 bp, Artificial Sequence
  Agr2 primer 4
aagatgagtgagccaaacca

SEQ ID NO: 21 - DNA, 20 bp, Artificial Sequence
  Agr2 primer 5
ggagtgaaggcagtcaacag

SEQ ID NO: 22 - DNA, 20 bp, Artificial Sequence
  Agr2 primer 6
gatgggacttggaggagatt

SEQ ID NO: 23 - DNA, 20 bp, Artificial Sequence
  Agr2 primer 7
tctgtagccccctctctctt

SEQ ID NO: 24 - DNA, 20 bp, Artificial Sequence
  Agr2 primer 8
cactaagtcccaccgagaaa

SEQ ID NO: 25 - DNA, 20 bp, Artificial Sequence
  Agr2 primer 9
gctggggtaggagataggag

SEQ ID NO: 26 - DNA, 20 bp, Artificial Sequence
  Agr2 primer 10
atcttgcccaacttcagtca

SEQ ID NO: 27 - DNA, 20 bp, Artificial Sequence
  Agr2 primer 11
taagcaggaagcaggagaga

SEQ ID NO: 28 - DNA, 20 bp, Artificial Sequence
  Agr2 primer 12
aatattgtttccccacctgt

SEQ ID NO: 29 - DNA, 1701 bp, Homo sapiens
ccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagaragagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagttttagaaatttggtttcaagtgtacatgtgtgaaaacaatattgtatactaccatagtgagccatgattttctaaaaaaaaaaataaatgttttgggggtgttctgttttctccaacttggtctttcacagtggttcgtttaccaaataggattaaacacacacaaaatgctcaaggaagggacaagacaaaaccaaaactagttcaaatgatgaagaccaaagaccaagttatcatctcaccacaccacaggttctcactagatgactgtaagtagacacgagcttaatcaacagaagtatcaagccatgtgctttagcataaaagaatatttagaaaaacatcccaagaaaatcacatcactacctagagtcaactctggccaggaactctaaggtacacactttcatttagtaattaaattttagtcagattttgcccaacctaatgctctcagggaaagcctctggcaagtagctttctccttcagaggtctaatttagtagaaaggtcatccaaagaacatctgcactcctgaacacaccctgaagaaatcctgggaattgaccttgtaatcgatttgtctgtcaaggtcctaaagtactggagtgaaataaattcagccaacatgtgactaattggaagaagagcaaagggtggtgacgtgttgatgaggcagatggagatcagaggttactagggtttaggaaacgtgaaaggctgtggcatcagggtaggggagcattctgcctaacagaaattagaattgtgtgttaatgtcttcactctatacttaatctcacattcattaatatatggaattcctctactgcccagcccctcctgatttctttggcccctggactatggtgctgtatataatgctttgcagtatctgttgcttgtcttgattaacttttttggataaaaccttttttgaacagaaaaaaaaaaaaaaaaaaaa

SEQ ID NO: 30 - protein, 175 aa, Homo sapiens
MEKIPVSAFLLLVALSYTLARDTTVKPGAKKDTKDSRPKLPQTLSRGWGDQLIWTQTYEEALYKSKTSNKPLMIIHHLDECPHSQALKKVFAENKEIQKLAEQFVLLNLVYETTDKHLSPDGQYVPRIMFVDPSLTERADITGRYSNRLYAYEPADTALLLDNMKKALKLLKTEL

SEQ ID NO: 31 - DNA, 23 bp, Artificial Sequence
  hAgr2-3 primer
gccatggagaaaattccagtgtc

SEQ ID NO: 32 - DNA, 26 bp, Artificial Sequence
  hAgr2-4 primer 1
tttacaattcagtcttcagcaacttg

SEQ ID NO: 33 - DNA, 531 bp, Artificial Sequence
  PCR product of hAgr2-3 and hAgr2-4
ccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaa

SEQ ID NO: 34 - protein, 19 aa, Artificial Sequence
  Agr2 epitope exon 11
TVKSGAKKDPKDSRPKLPQ